SEQUENCE LISTING (111 sequences)

SEQ ID NO: 1 - protein, 265 aa, Homo sapiens
MSEEVTYADLQFQNSSEMEKIPEIGKFGEKAPPAPSHVWRPAALFLTLLCLLLLIGLGVLASMFHVTLKIEMKKMNKLQNISEELQRNISLQLMSNMNISNKIRNLSTTLQTIATKLCRELYSKEQEHKCKPCPRRWIWHKDSCYFLSDDVQTWQESKMACAAQNASLLKINNKNALEFIKSQSRSYDYWLGLSPEEDSTRGMRVDNIINSSAWVIRNAPDLNNMYCGYINRLYVQYYHCTYKKRMICEKMANPVQLGSTYFREA

SEQ ID NO: 2 - DNA, 798 bp, Homo sapiens
  source 1..798: mol_type = genomic DNA
atgtctgaagaagttacttatgcagatcttcaattccagaactccagtgagatggaaaaaatcccagaaattggcaaatttggggaaaaagcacctccagctccctctcatgtatggcgtccagcagccttgtttctgactcttctgtgccttctgttgctcattggattgggagtcttggcaagcatgtttcacgtaactttgaagatagaaatgaaaaaaatgaacaaactacaaaacatcagtgaagagctccagagaaatatttctctacaactgatgagtaacatgaatatctccaacaagatcaggaacctctccaccacactgcaaacaatagccaccaaattatgtcgtgagctatatagcaaagaacaagagcacaaatgtaagccttgtccaaggagatggatttggcataaggacagctgttatttcctaagtgatgatgtccaaacatggcaggagagtaaaatggcctgtgctgctcagaatgccagcctgttgaagataaacaacaaaaatgcattggaatttataaaatcccagagtagatcatatgactattggctgggattatctcctgaagaagattccactcgtggtatgagagtggataatataatcaactcctctgcctgggttataagaaacgcacctgacttaaataacatgtattgtggatatataaatagactatatgttcaatattatcactgcacttataaaaaaagaatgatatgtgagaagatggccaatccagtgcagcttggttctacatattttagggaggcatga

SEQ ID NO: 3 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 4 - DNA, 1092 bp, Homo sapiens
  source 1..1092: mol_type = genomic DNA
atgccgctgctgctactgctgcccctgctgtgggcaggggccctggctatggatccaaatttctggctgcaagtgcaggagtcagtgacggtacaggagggtttgtgcgtcctcgtgccctgcactttcttccatcccataccctactacgacaagaactccccagttcatggttactggttccgggaaggagccattatatccagggactctccagtggccacaaacaagctagatcaagaagtacaggaggagactcagggcagattccgcctccttggggatcccagtaggaacaactgctccctgagcatcgtagacgccaggaggagggataatggttcatacttctttcggatggagagaggaagtaccaaatacagttacaaatctccccagctctctgtgcatgtgacagacttgacccacaggcccaaaatcctcatccctggcactctagaacccggccactccaaaaacctgacctgctctgtgtcctgggcctgtgagcagggaacacccccgatcttctcctggttgtcagctgcccccacctccctgggccccaggactactcactcctcggtgctcataatcaccccacggccccaggaccacggcaccaacctgacctgtcaggtgaagttcgctggagctggtgtgactacggagagaaccatccagctcaacgtcacctatgttccacagaacccaacaactggtatctttccaggagatggctcagggaaacaagagaccagagcaggagtggttcatggggccattggaggagctggtgttacagccctgctcgctctttgtctctgcctcatcttcttcatagtgaagacccacaggaggaaagcagccaggacagcagtgggcaggaatgacacccaccctaccacagggtcagcctccccgaaacaccagaagaagtccaagttacatggccccactgaaacctcaagctgttcaggtgccgcccctactgtggagatggatgaggagctgcattatgcttccctcaactttcatgggatgaatccttccaaggacacctccaccgaatactcagaggtcaggacccag

SEQ ID NO: 5 - protein, 113 aa, synthetic construct
EVQLVESGGGLVQPGGSLRLSCAASGFTFSTYDMSWVRQAPGKQREWVSAISGSGGTTYYADSVKGRFTISRDNSKNTLYLQMNSLRAEDTAVYYCVRGDDHWGQGTLVTVSS

SEQ ID NO: 6 - protein, 115 aa, synthetic construct
EVQLVESGGGLVQPGGSLRLSCAASGFTFSNYDMSWVRQAPGKQLEWVSAITGSGGSTYYADSVKGRFTISRDNSKNTLYLQMNSLRAEDTAVYYCAAGVDGDYWGQGTLVTVSS

SEQ ID NO: 7 - protein, 122 aa, synthetic construct
EVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSS

SEQ ID NO: 8 - protein, 8 aa, synthetic construct
GFTFSTYD

SEQ ID NO: 9 - protein, 8 aa, synthetic construct
GFTFSNYD

SEQ ID NO: 10 - protein, 8 aa, synthetic construct
GFTFSAYD

SEQ ID NO: 11 - protein, 8 aa, synthetic construct
ISGSGGTT

SEQ ID NO: 12 - protein, 8 aa, synthetic construct
ITGSGGST

SEQ ID NO: 13 - protein, 8 aa, synthetic construct
ISGSGGST

SEQ ID NO: 14 - protein, 6 aa, synthetic construct
VRGDDH

SEQ ID NO: 15 - protein, 8 aa, synthetic construct
AAGVDGDY

SEQ ID NO: 16 - protein, 15 aa, synthetic construct
AYDGDSTGSLDEMAH

SEQ ID NO: 17 - protein, 118 aa, synthetic construct
EVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSS

SEQ ID NO: 18 - protein, 118 aa, synthetic construct
EVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSS

SEQ ID NO: 19 - protein, 8 aa, synthetic construct
GFTFSTYA

SEQ ID NO: 20 - protein, 8 aa, synthetic construct
GFTFSYYA

SEQ ID NO: 21 - protein, 8 aa, synthetic construct
IEGSGGST

SEQ ID NO: 22 - protein, 8 aa, synthetic construct
IEGSGGRT

SEQ ID NO: 23 - protein, 11 aa, synthetic construct
AVETGWADFDY

SEQ ID NO: 24 - protein, 11 aa, synthetic construct
VKETGWADFDY

SEQ ID NO: 25 - protein, 367 aa, synthetic construct
MALPVTALLLPLALLLHAARPEVQLVESGGGLVQPGGSLRLSCAASGFTFSTYDMSWVRQAPGKQREWVSAISGSGGTTYYADSVKGRFTISRDNSKNTLYLQMNSLRAEDTAVYYCVRGDDHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - protein, 369 aa, synthetic construct
MALPVTALLLPLALLLHAARPEVQLVESGGGLVQPGGSLRLSCAASGFTFSNYDMSWVRQAPGKQLEWVSAITGSGGSTYYADSVKGRFTISRDNSKNTLYLQMNSLRAEDTAVYYCAAGVDGDYWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 27 - protein, 376 aa, synthetic construct
MALPVTALLLPLALLLHAARPEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - protein, 372 aa, synthetic construct
MALPVTALLLPLALLLHAARPEVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 29 - protein, 372 aa, synthetic construct
MALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 30 - protein, 366 aa, synthetic construct
MALPVTALLLPLALLLHAARPEVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 31 - protein, 366 aa, synthetic construct
MALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 32 - protein, 113 aa, synthetic construct
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - protein, 42 aa, synthetic construct
KRGRKKLLYIFKQPFMRPVQTTQEEDGCSCRFPEEEEGGCEL

SEQ ID NO: 34 - protein, 78 aa, synthetic construct
TTTPAPRPPTPAPTIASQPLSLRPEACRPAAGGAVHTRGLDFACDIYIWAPLAGTCGVLLLSLVITLYCNHRNRRRVC

SEQ ID NO: 35 - protein, 72 aa, synthetic construct
TQPQETQGPPARPITVQPTEAWPRTSQGPSTRPVEVPGGRAVAAILGLGLVLGLLGPLAILLALYLLRRDQR

SEQ ID NO: 36 - protein, 15 aa, synthetic construct
GGGGSGGGGSGGGGS

SEQ ID NO: 37 - protein, 20 aa, synthetic construct
GGGGSGGGGSGGGGSGGGGS

SEQ ID NO: 38 - protein, 5 aa, synthetic construct
GGGGS

SEQ ID NO: 39 - protein, 21 aa, synthetic construct
MALPVTALLLPLALLLHAARP

SEQ ID NO: 40 - protein, 500 aa, synthetic construct
MALPVTALLLPLALLLHAARPEVQLVESGGGLVQPGGSLRLSCAASGFTFSTYDMSWVRQAPGKQREWVSAISGSGGTTYYADSVKGRFTISRDNSKNTLYLQMNSLRAEDTAVYYCVRGDDHWGQGTLVTVSSGGGGSGGGGSGGGGSEVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 41 - protein, 502 aa, synthetic construct
MALPVTALLLPLALLLHAARPEVQLVESGGGLVQPGGSLRLSCAASGFTFSNYDMSWVRQAPGKQLEWVSAITGSGGSTYYADSVKGRFTISRDNSKNTLYLQMNSLRAEDTAVYYCAAGVDGDYWGQGTLVTVSSGGGGSGGGGSGGGGSEVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 42 - protein, 509 aa, synthetic construct
MALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSGGGGSGGGGSGGGGSEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 43 - protein, 18 aa, synthetic construct
EGRGSLLTCGDVEENPGP

SEQ ID NO: 44 - protein, 751 aa, synthetic construct
MALPVTALLLPLALLLHAARPEVQLVESGGGLVQPGGSLRLSCAASGFTFSTYDMSWVRQAPGKQREWVSAISGSGGTTYYADSVKGRFTISRDNSKNTLYLQMNSLRAEDTAVYYCVRGDDHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREGRGSLLTCGDVEENPGPMALPVTALLLPLALLLHAARPEVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 45 - protein, 753 aa, synthetic construct
MALPVTALLLPLALLLHAARPEVQLVESGGGLVQPGGSLRLSCAASGFTFSNYDMSWVRQAPGKQLEWVSAITGSGGSTYYADSVKGRFTISRDNSKNTLYLQMNSLRAEDTAVYYCAAGVDGDYWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREGRGSLLTCGDVEENPGPMALPVTALLLPLALLLHAARPEVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 46 - protein, 760 aa, synthetic construct
MALPVTALLLPLALLLHAARPEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREGRGSLLTCGDVEENPGPMALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 47 - protein, 244 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIK

SEQ ID NO: 48 - protein, 242 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 49 - protein, 122 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSS

SEQ ID NO: 50 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 51 - protein, 8 aa, synthetic construct
GGSISSYY

SEQ ID NO: 52 - protein, 8 aa, synthetic construct
GYTFTDYN

SEQ ID NO: 53 - protein, 7 aa, synthetic construct
IYYSGST

SEQ ID NO: 54 - protein, 8 aa, synthetic construct
IYPYNGGT

SEQ ID NO: 55 - protein, 16 aa, synthetic construct
VSLVYCGGDCYSGFDY

SEQ ID NO: 56 - protein, 9 aa, synthetic construct
ARGRPAMDY

SEQ ID NO: 57 - protein, 107 aa, synthetic construct
DIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIK

SEQ ID NO: 58 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 59 - protein, 6 aa, synthetic construct
QDINNF

SEQ ID NO: 60 - protein, 10 aa, synthetic construct
ESVDNYGISF

SEQ ID NO: 61-62 - skipped (2 entries)

SEQ ID NO: 63 - protein, 9 aa, synthetic construct
QQYGNLPFT

SEQ ID NO: 64 - protein, 9 aa, synthetic construct
QQSKEVPWT

SEQ ID NO: 65 - protein, 498 aa, synthetic construct
MALPVTALLLPLALLLHAARPQVQLQESGPGLVKPSETLSLTCTVSGGSISSYYWSWIRQPPGKGLEWIGYIYYSGSTNYNPSLKSRVTISVDTSKNQFSLKLSSVTAADTAVYYCVSLVYCGGDCYSGFDYWGQGTLVTVSSGGGGSGGGGSGGGGSDIQLTQSPSSLSASVGDRVSFTCQASQDINNFLNWYQQKPGKAPKLLIYDASNLETGVPSRFSGSGSGTDFTFTISSLQPEDIATYYCQQYGNLPFTFGGGTKVEIKTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 66 - protein, 496 aa, synthetic construct
MALPVTALLLPLALLLHAARPQVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSSGGGGSGGGGSGGGGSDIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIKTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 67 - protein, 11 aa, synthetic construct
SGYTFTRYTMH

SEQ ID NO: 68 - protein, 17 aa, synthetic construct
YINPSRGYTNYNQKFKD

SEQ ID NO: 69 - protein, 8 aa, synthetic construct
YYDDHYCL

SEQ ID NO: 70 - protein, 10 aa, synthetic construct
SASSSVSYMN

SEQ ID NO: 71 - protein, 7 aa, synthetic construct
DTSKLAS

SEQ ID NO: 72 - protein, 8 aa, synthetic construct
QQWSSNPF

SEQ ID NO: 73 - DNA, 1101 bp, synthetic construct
  source 1..1101: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagcacatatgatatgagctgggtccgccaggctccagggaagcagcgtgagtgggtctcagctatttccggtagtggtggtacaacatactacgcagactccgtgaagggccggttcaccatctccagagacaattccaagaacacgctgtatctgcaaatgaacagcctgagagccgaggacacggccgtatattactgtgtgcgcggcgacgaccattggggccagggaaccctcgtgacagttagcagcactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 74 - DNA, 1107 bp, synthetic construct
  source 1..1107: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagcaactatgacatgagctgggtccgccaggctccagggaagcagcttgagtgggtctcagctattaccggtagtggtggtagtacatactacgcagactccgtgaagggccggttcaccatctccagagacaattccaagaacacgctgtatctgcaaatgaacagcctgagagccgaggacacggccgtatattactgtgctgcaggcgtagatggcgactattggggccagggaaccctcgtgacagttagcagcactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 75 - DNA, 1128 bp, synthetic construct
  source 1..1128: mol_type = other DNA
atggcccttcccgttaccgccttactcttacctcttgctttattactgcacgcggcacggcctgaggtgcagctggtggagtctgggggaggcttggtacagcctggggggtccctgagactctcctgtgcagcctctggattcacctttagcgcgtatgatatgagctgggtccgccaggctccagggaagcagcttgagtgggtctcagctattagcggtagtggtggtagcacatactacgcagactccgtgaagggccggttcaccatctccagagacaattccaagaacacgctgtatctgcaaatgaacagcctgagagccgaggacacggccgtatattactgtgcctacgatggtgattctacagggtctctggatgagatggcgcactggggccagggaaccctggtcaccgtttcatctactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 76 - DNA, 1116 bp, synthetic construct
  source 1..1116: mol_type = other DNA
atggcccttcccgttaccgccttactcttacctcttgctttattactgcacgcggcacggcctgaggtgcagctggtggagtctgggggaggcttggtacagcctggggggtccctgagactctcctgtgcagcctctggattcacctttagcacgtatgccatgagctgggtccgccaggctccagggaagcagcgtgagtgggtctcagctattgaaggtagtggtggttctacatactacgcagactccgtgaagggccggttcaccatctccagagacaattccaagaacacgctgtatctgcaaatgaacagcctgagagccgaggacacggccgtatattactgtgccgtagagacaggatgggcggattttgactattggggccagggaaccctggtcaccgtttcatctactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 77 - DNA, 1116 bp, synthetic construct
  source 1..1116: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagctactatgccatgagctgggtccgccaggctccagggaagcagcgtgagtgggtctcagctattgaaggtagtggtggtcggacatactacgcagactccgtgaagggccggttcaccatctccagagacaattccaagaacacgctgtatctgcaaatgaacagcctgagagccgaggacacggccgtatattactgtgttaaagagacaggatgggcggattttgactattggggccagggaaccctcgtgacagttagcagcactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 78 - DNA, 1098 bp, synthetic construct
  source 1..1098: mol_type = other DNA
atggcccttcccgtgacggctcttcttttgccactggcacttcttttgcacgcagcgcgacctgaggtgcagctggtggagtctgggggaggcttggtacagcctggggggtccctgagactctcctgtgcagcctctggattcacctttagcacgtatgccatgagctgggtccgccaggctccagggaagcagcgtgagtgggtctcagctattgaaggtagtggtggttctacatactacgcagactccgtgaagggccggttcaccatctccagagacaattccaagaacacgctgtatctgcaaatgaacagcctgagagccgaggacacggccgtatattactgtgccgtagagacaggatgggcggattttgactattggggccagggaaccctggtcaccgtttcatctacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctgcggagggaccagaggaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 79 - DNA, 1098 bp, synthetic construct
  source 1..1098: mol_type = other DNA
atggcccttcccgtgacggctcttcttttgccactggcacttcttttgcacgcagcgcgacctgaagtccaactcgtcgaaagcgggggaggcttggtacagcctggggggtccctgagactctcctgtgcagcctctggattcacctttagctactatgccatgagctgggtccgccaggctccagggaagcagcgtgagtgggtctcagctattgaaggtagtggtggtcggacatactacgcagactccgtgaagggccggttcaccatctccagagacaattccaagaacacgctgtatctgcaaatgaacagcctgagagccgaggacacggccgtatattactgtgttaaagagacaggatgggcggattttgactattggggccagggaaccctcgtgacagttagcagcacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctgcggagggaccagaggaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 80 - DNA, 1500 bp, synthetic construct
  source 1..1500: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagcacatatgatatgagctgggtccgccaggctccagggaagcagcgtgagtgggtctcagctatttccggtagtggtggtacaacatactacgcagactccgtgaagggccggttcaccatctccagagacaattccaagaacacgctgtatctgcaaatgaacagcctgagagccgaggacacggccgtatattactgtgtgcgcggcgacgaccattggggccagggaaccctcgtgacagttagcagcggcggagggggatctggaggtggaggatcagggggaggtggttccgaggtgcagctggtggagtctgggggaggcttggtacagcctggggggtccctgagactctcctgtgcagcctctggattcacctttagcacgtatgccatgagctgggtccgccaggctccagggaagcagcgtgagtgggtctcagctattgaaggtagtggtggttctacatactacgcagactccgtgaagggccggttcaccatctccagagacaattccaagaacacgctgtatctgcaaatgaacagcctgagagccgaggacacggccgtatattactgtgccgtagagacaggatgggcggattttgactattggggccagggaaccctggtcaccgtttcatctactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 81 - DNA, 1506 bp, synthetic construct
  source 1..1506: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagcaactatgacatgagctgggtccgccaggctccagggaagcagcttgagtgggtctcagctattaccggtagtggtggtagtacatactacgcagactccgtgaagggccggttcaccatctccagagacaattccaagaacacgctgtatctgcaaatgaacagcctgagagccgaggacacggccgtatattactgtgctgcaggcgtagatggcgactattggggccagggaaccctcgtgacagttagcagcggcggagggggatctggaggtggaggatcagggggaggtggttccgaggtgcagctggtggagtctgggggaggcttggtacagcctggggggtccctgagactctcctgtgcagcctctggattcacctttagcacgtatgccatgagctgggtccgccaggctccagggaagcagcgtgagtgggtctcagctattgaaggtagtggtggttctacatactacgcagactccgtgaagggccggttcaccatctccagagacaattccaagaacacgctgtatctgcaaatgaacagcctgagagccgaggacacggccgtatattactgtgccgtagagacaggatgggcggattttgactattggggccagggaaccctggtcaccgtttcatctactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 82 - DNA, 1527 bp, synthetic construct
  source 1..1527: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagctactatgccatgagctgggtccgccaggctccagggaagcagcgtgagtgggtctcagctattgaaggtagtggtggtcggacatactacgcagactccgtgaagggccggttcaccatctccagagacaattccaagaacacgctgtatctgcaaatgaacagcctgagagccgaggacacggccgtatattactgtgttaaagagacaggatgggcggattttgactattggggccagggaaccctcgtgacagttagcagcggcggagggggatctggaggtggaggatcagggggaggtggttccgaggtgcagctggtggagtctgggggaggcttggtacagcctggggggtccctgagactctcctgtgcagcctctggattcacctttagcgcgtatgatatgagctgggtccgccaggctccagggaagcagcttgagtgggtctcagctattagcggtagtggtggtagcacatactacgcagactccgtgaagggccggttcaccatctccagagacaattccaagaacacgctgtatctgcaaatgaacagcctgagagccgaggacacggccgtatattactgtgcctacgatggtgattctacagggtctctggatgagatggcgcactggggccagggaaccctggtcaccgtttcatctactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 83 - DNA, 2253 bp, synthetic construct
  source 1..2253: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagcacatatgatatgagctgggtccgccaggctccagggaagcagcgtgagtgggtctcagctatttccggtagtggtggtacaacatactacgcagactccgtgaagggccggttcaccatctccagagacaattccaagaacacgctgtatctgcaaatgaacagcctgagagccgaggacacggccgtatattactgtgtgcgcggcgacgaccattggggccagggaaccctcgtgacagttagcagcactacgactcctgcaccgcgaccgcccacaccagctcctaccatagcttcccaacctctctctcttcgcccagaagcgtgtcggcctgccgcgggtggcgcagtacacacgaggggcctggactttgcatgtgatatctacatctgggctccactcgcaggaacctgtggcgtattgcttctttccttggtgattactctctattgcaatcacagaaataggaggcgagtgtgtaaacgcgggcgcaagaagttgttgtacatattcaagcaaccctttatgagaccagtgcaaacaacacaagaagaggatggttgttcatgcagattccctgaggaggaagagggcggctgcgaacttagggtcaagttttcaaggtccgctgacgcaccggcataccagcaaggccaaaatcagctttataatgaactgaatctcgggcgccgagaagaatatgatgttcttgacaagaggcgcgggagggaccccgaaatggggggcaaaccccagcggaggaagaatccccaggaaggcctttacaatgaattgcagaaagacaaaatggctgaagcatacagtgaaataggaatgaagggggagaggcgccgcgggaagggacatgacggattgtatcagggattgagtactgcgacgaaagacacgtacgatgctcttcacatgcaagcgcttccaccgcgggagggcagaggaagtcttctaacatgcggtgacgtggaggagaatcccggccctatggcccttcccgtgacggctcttcttttgccactggcacttcttttgcacgcagcgcgacctgaggtgcagctggtggagtctgggggaggcttggtacagcctggggggtccctgagactctcctgtgcagcctctggattcacctttagcacgtatgccatgagctgggtccgccaggctccagggaagcagcgtgagtgggtctcagctattgaaggtagtggtggttctacatactacgcagactccgtgaagggccggttcaccatctccagagacaattccaagaacacgctgtatctgcaaatgaacagcctgagagccgaggacacggccgtatattactgtgccgtagagacaggatgggcggattttgactattggggccagggaaccctggtcaccgtttcatctacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctgcggagggaccagaggaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 84 - DNA, 2259 bp, synthetic construct
  source 1..2259: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagcaactatgacatgagctgggtccgccaggctccagggaagcagcttgagtgggtctcagctattaccggtagtggtggtagtacatactacgcagactccgtgaagggccggttcaccatctccagagacaattccaagaacacgctgtatctgcaaatgaacagcctgagagccgaggacacggccgtatattactgtgctgcaggcgtagatggcgactattggggccagggaaccctcgtgacagttagcagcactacgactcctgcaccgcgaccgcccacaccagctcctaccatagcttcccaacctctctctcttcgcccagaagcgtgtcggcctgccgcgggtggcgcagtacacacgaggggcctggactttgcatgtgatatctacatctgggctccactcgcaggaacctgtggcgtattgcttctttccttggtgattactctctattgcaatcacagaaataggaggcgagtgtgtaaacgcgggcgcaagaagttgttgtacatattcaagcaaccctttatgagaccagtgcaaacaacacaagaagaggatggttgttcatgcagattccctgaggaggaagagggcggctgcgaacttagggtcaagttttcaaggtccgctgacgcaccggcataccagcaaggccaaaatcagctttataatgaactgaatctcgggcgccgagaagaatatgatgttcttgacaagaggcgcgggagggaccccgaaatggggggcaaaccccagcggaggaagaatccccaggaaggcctttacaatgaattgcagaaagacaaaatggctgaagcatacagtgaaataggaatgaagggggagaggcgccgcgggaagggacatgacggattgtatcagggattgagtactgcgacgaaagacacgtacgatgctcttcacatgcaagcgcttccaccgcgggagggcagaggaagtcttctaacatgcggtgacgtggaggagaatcccggccctatggcccttcccgtgacggctcttcttttgccactggcacttcttttgcacgcagcgcgacctgaggtgcagctggtggagtctgggggaggcttggtacagcctggggggtccctgagactctcctgtgcagcctctggattcacctttagcacgtatgccatgagctgggtccgccaggctccagggaagcagcgtgagtgggtctcagctattgaaggtagtggtggttctacatactacgcagactccgtgaagggccggttcaccatctccagagacaattccaagaacacgctgtatctgcaaatgaacagcctgagagccgaggacacggccgtatattactgtgccgtagagacaggatgggcggattttgactattggggccagggaaccctggtcaccgtttcatctacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctgcggagggaccagaggaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 85 - DNA, 2280 bp, synthetic construct
  source 1..2280: mol_type = other DNA
atggcccttcccgttaccgccttactcttacctcttgctttattactgcacgcggcacggcctgaggtgcagctggtggagtctgggggaggcttggtacagcctggggggtccctgagactctcctgtgcagcctctggattcacctttagcgcgtatgatatgagctgggtccgccaggctccagggaagcagcttgagtgggtctcagctattagcggtagtggtggtagcacatactacgcagactccgtgaagggccggttcaccatctccagagacaattccaagaacacgctgtatctgcaaatgaacagcctgagagccgaggacacggccgtatattactgtgcctacgatggtgattctacagggtctctggatgagatggcgcactggggccagggaaccctggtcaccgtttcatctactacgactcctgcaccgcgaccgcccacaccagctcctaccatagcttcccaacctctctctcttcgcccagaagcgtgtcggcctgccgcgggtggcgcagtacacacgaggggcctggactttgcatgtgatatctacatctgggctccactcgcaggaacctgtggcgtattgcttctttccttggtgattactctctattgcaatcacagaaataggaggcgagtgtgtaaacgcgggcgcaagaagttgttgtacatattcaagcaaccctttatgagaccagtgcaaacaacacaagaagaggatggttgttcatgcagattccctgaggaggaagagggcggctgcgaacttagggtcaagttttcaaggtccgctgacgcaccggcataccagcaaggccaaaatcagctttataatgaactgaatctcgggcgccgagaagaatatgatgttcttgacaagaggcgcgggagggaccccgaaatggggggcaaaccccagcggaggaagaatccccaggaaggcctttacaatgaattgcagaaagacaaaatggctgaagcatacagtgaaataggaatgaagggggagaggcgccgcgggaagggacatgacggattgtatcagggattgagtactgcgacgaaagacacgtacgatgctcttcacatgcaagcgcttccaccgcgggagggcagaggaagtcttctaacatgcggtgacgtggaggagaatcccggccctatggcccttcccgtgacggctcttcttttgccactggcacttcttttgcacgcagcgcgacctgaagtccaactcgtcgaaagcgggggaggcttggtacagcctggggggtccctgagactctcctgtgcagcctctggattcacctttagctactatgccatgagctgggtccgccaggctccagggaagcagcgtgagtgggtctcagctattgaaggtagtggtggtcggacatactacgcagactccgtgaagggccggttcaccatctccagagacaattccaagaacacgctgtatctgcaaatgaacagcctgagagccgaggacacggccgtatattactgtgttaaagagacaggatgggcggattttgactattggggccagggaaccctcgtgacagttagcagcacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctgcggagggaccagaggaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 86 - DNA, 1494 bp, synthetic construct
  source 1..1494: mol_type = other DNA
atggcccttcccgttaccgccttactcttacctcttgctttattactgcacgcggcacggcctcaggtccaactgcaagaaagcggacccggactggtgaagccttctgagacacttagtctgacgtgcacggtcagtggcggctccatctcctcctattattggtcatggatacgacaacccccaggtaagggcctggaatggattggctatatctactattcaggaagcacgaactacaatcccagcctgaagtcccgagtgacaatttcagtagataccagtaaaaaccagttcagtcttaaactgtcaagcgtgacagctgccgacaccgctgtgtattactgcgtcagcctggtgtattgtggaggggattgttatagcgggttcgattattggggacagggaaccctggtgactgtatcttccggcggcggcggctcagggggtggcggtagtggcggtgggggttccgatattcaactgacacaatcccccagctcactcagcgccagcgtgggggacagggttagctttacctgtcaagcctctcaggatataaataactttctgaactggtatcaacagaagcctgggaaggcgcccaaactcctgatctatgatgcgtccaacctggaaactggcgtgccttcacgctttagcggctctggcagtggtacagacttcacttttaccatctcttcacttcagccggaggacatcgccacatattactgtcaacagtacggaaacttgccctttacttttggaggcggcaccaaagttgaaatcaaaactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 87 - DNA, 1488 bp, synthetic construct
  source 1..1488: mol_type = other DNA
atggcccttcccgttaccgccttactcttacctcttgctttattactgcacgcggcacggcctcaagtgcaactggtacagagcggggctgaagtgaagaagccgggaagcagcgtcaaggttagctgtaaggcatctggctacactttcactgattataacatgcactgggtgagacaagcaccgggtcagggcctcgaatggatcgggtatatttatccgtataacgggggcaccgggtataatcagaagtttaagagcaaggccactataacggccgacgaatcaaccaatacggcctatatggagctgtcaagccttagatcagaggacactgccgtctactattgtgcaagaggtagacctgcaatggattattggggacagggtaccttggtgacggtatctagtggcggagggggatctggaggtggaggatcagggggaggtggttccgatatccaaatgactcaatccccctcttcactgagtgcctcagttggagaccgcgttacaatcacatgtagagcctcagagtcagtggataattatgggattagcttcatgaactggttccagcaaaagccaggtaaggcgcctaaactcctgatttatgcggcatccaaccaaggcagtggggttccgtcacgcttctccgggtcagggtccggcacggacttcacactgacgatctcatcattgcagccggatgacttcgcaacgtattattgccagcagtcaaaagaagttccctggaccttcggtcagggtaccaaagtcgaaatcaagactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 88 - protein, 21 aa, synthetic construct
GSGEGRGSLLTCGDVEENPGP

SEQ ID NO: 89 - protein, 43 aa, synthetic construct
GSGATNFSLLKQAGDVEENPGPGSGEGRGSLLTCGDVEENPGP

SEQ ID NO: 90 - protein, 27 aa, synthetic construct
ARAKRSGSGEGRGSLITCGDVEENPGP

SEQ ID NO: 91 - protein, 22 aa, synthetic construct
GSGATNFSLLKQAGDVEENPGP

SEQ ID NO: 92 - protein, 316 aa, synthetic construct
MPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 93 - protein, 509 aa, synthetic construct
MALPVTALLLPLALLLHAARPEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSGGGGSGGGGSGGGGSEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 94 - protein, 499 aa, synthetic construct
MALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSGGGGSEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 95 - protein, 763 aa, synthetic construct
MALPVTALLLPLALLLHAARPEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 96 - protein, 785 aa, synthetic construct
MALPVTALLLPLALLLHAARPEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPGSGEGRGSLLTCGDVEENPGPMALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 97 - DNA, 1527 bp, synthetic construct
  source 1..1527: mol_type = other DNA
atggcccttcccgttaccgccttactcttacctcttgctttattactgcacgcggcacggcctgaagtccagttggtcgaatccggcggtggcctcgtgcagccaggcggttcactgagactttcatgtgctgcttcagggtttaccttttcatactatgctatgtcatgggtacgacaagctccaggaaaacaacgcgagtgggtttcagcgatagaaggtagcggcggcagaacgtattatgccgactctgtgaaggggcgattcacgattagtcgagacaattccaagaacactttgtatctgcagatgaatagcctccgcgccgaagatacggccgtgtactactgcgtaaaagaaaccggctgggcagactttgattactggggtcaaggcacactggtgactgtatcatctggtggtggtggttctggagggggtgggagcggaggtggaggaagtgaagttcagttggtggagagcggtggaggccttgtccagccgggtgggtctttgcggttgagttgcgctgcatccggtttcaccttctcagcctatgatatgtcttgggttaggcaagctccgggcaaacaattggaatgggtgtcagcaatatccggttcaggtgggtccacatactacgcggattctgttaagggacgatttaccattagcagagacaatagtaaaaacactctgtacttgcagatgaatagtcttcgcgcggaggataccgccgtgtactattgtgcatatgatggcgacagcacgggctctcttgacgaaatggcccactggggacaagggacgttggttacggtgtcaagtactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 98 - DNA, 1527 bp, synthetic construct
  source 1..1527: mol_type = other DNA
atggcccttcccgttaccgccttactcttacctcttgctttattactgcacgcggcacggcctgaagttcagttggtggagagcggtggaggccttgtccagccgggtgggtctttgcggttgagttgcgctgcatccggtttcaccttctcagcctatgatatgtcttgggttaggcaagctccgggcaaacaattggaatgggtgtcagcaatatccggttcaggtgggtccacatactacgcggattctgttaagggacgatttaccattagcagagacaatagtaaaaacactctgtacttgcagatgaatagtcttcgcgcggaggataccgccgtgtactattgtgcatatgatggcgacagcacgggctctcttgacgaaatggcccactggggacaagggacgttggttacggtgtcaagtggtggtggtggttctggagggggtgggagcggaggtggaggaagtgaagtccagttggtcgaatccggcggtggcctcgtgcagccaggcggttcactgagactttcatgtgctgcttcagggtttaccttttcatactatgctatgtcatgggtacgacaagctccaggaaaacaacgcgagtgggtttcagcgatagaaggtagcggcggcagaacgtattatgccgactctgtgaaggggcgattcacgattagtcgagacaattccaagaacactttgtatctgcagatgaatagcctccgcgccgaagatacggccgtgtactactgcgtaaaagaaaccggctgggcagactttgattactggggtcaaggcacactggtgactgtatcatctactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 99 - DNA, 1497 bp, synthetic construct
  source 1..1497: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagctactatgccatgagctgggtccgccaggctccagggaagcagcgtgagtgggtctcagctattgaaggtagtggtggtcggacatactacgcagactccgtgaagggccggttcaccatctccagagacaattccaagaacacgctgtatctgcaaatgaacagcctgagagccgaggacacggccgtatattactgtgttaaagagacaggatgggcggattttgactattggggccagggaaccctcgtgacagttagcagcgggggaggtggttccgaggtgcagctggtggagtctgggggaggcttggtacagcctggggggtccctgagactctcctgtgcagcctctggattcacctttagcgcgtatgatatgagctgggtccgccaggctccagggaagcagcttgagtgggtctcagctattagcggtagtggtggtagcacatactacgcagactccgtgaagggccggttcaccatctccagagacaattccaagaacacgctgtatctgcaaatgaacagcctgagagccgaggacacggccgtatattactgtgcctacgatggtgattctacagggtctctggatgagatggcgcactggggccagggaaccctggtcaccgtttcatctactaccacaccagctcccaggccacccacgcctgcaccgacaatcgcatctcaaccactctcactccgccccgaagcgtgcagacccgcagcaggtggagccgtacatactaggggtcttgacttcgcctgtgatatatatatttgggcgcccttggccgggacttgtggggtccttctcctgtcactggttatcaccctttactgcaaccacaggaaccgaagacgtgtttgcaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 100 - DNA, 2289 bp, synthetic construct
  source 1..2289: mol_type = other DNA
atggcgctccccgtgaccgcgttgcttctcccgctggcactcctgttgcatgcggcacgaccggaagttcagctggtggagagcggaggaggcctcgtccaacctgggggctcacttaggttgtcttgcgcggcgagcggcttcaccttttcagcatatgatatgagttgggtaagacaagccccaggaaaacagctcgaatgggtatctgctataagtggatccggagggtcaacttactacgcggactctgttaaggggcgctttacaattagccgcgacaacagtaagaacactttgtaccttcagatgaactcacttcgggccgaggacacagctgtatattactgtgcatatgatggggattctactggctctctggatgagatggcccactggggtcaaggcacactcgtgacggtttcctcaaccaccacgccagcgcctagaccccctactccggcccctacaatagcaagtcaacccctctctctgagacctgaagcctgccgacccgcagcgggtggagctgtccacacccgcggcctggatttcgcatgtgacatatacatatgggcgcccctggcaggtacctgcggtgttctgttgcttagccttgtaattacgctttactgtaaccacagaaatcgacggagggtctgtaagcgcggtcggaagaagctcttgtacattttcaagcagccttttatgaggccggtccaaacgacacaagaggaagacggatgctcatgtaggtttccagaggaagaggaaggaggctgtgaacttcgggtcaaatttagtagatctgcggatgcaccggcatatcaacaagggcaaaatcaactgtacaacgagctcaatctgggtaggcgagaagaatatgacgtacttgataagcggcggggacgggatcctgaaatgggtggtaaaccacagcgcaggaaaaacccgcaggaggggctttataacgagcttcaaaaagacaaaatggcggaggcctactccgaaataggcatgaagggcgaaagacgacgcggtaaggggcacgatggcttgtaccaagggttgtccaccgcgactaaagacacatacgacgctcttcatatgcaggcgttgccgccgaggggttcaggcgaaggccggggcagtcttttgacatgcggcgatgttgaggagaaccctggacccatggcgcttcctgtaactgcactgcttctcccactcgcattgcttctgcatgcagcccgccccgaagttcaactcgtagaatccgggggaggcttggtccagccaggtggatccctccgactctcatgtgccgcaagtgggtttacgttcagttattacgcaatgtcatgggtgagacaggcaccagggaaacagagagaatgggtaagcgcaatcgagggatccggagggcggacctactacgccgactccgtcaagggacgcttcacgattagcagggacaacagtaagaacaccttgtacctgcaaatgaattcacttcgagctgaagacactgccgtttattattgcgtcaaggaaacgggctgggccgattttgattattggggacagggaacattggtcacggtgtcatcaacccagccgcaagagactcaagggccgcccgccaggccgataacggtacagcccactgaggcgtggccacgaacctctcagggcccatccactaggcccgtcgaagtacctggggggagagccgtcgctgccatcctggggctcggtctggttttgggcttgttgggtccgcttgcaattttgttggccttgtaccttctgcggagagaccagcggaagcggggtaggaaaaaacttctgtatatattcaagcaacccttcatgcgcccggtgcagaccacccaagaagaggatgggtgcagctgcaggtttccagaggaggaggaaggcggctgcgagttgcgggtaaagttttcaaggtcagcagatgctccggcctaccagcaaggacagaaccaattgtacaatgagctgaatttgggtcgccgagaagaatatgacgtcttggataagcgcagaggacgggacccagaaatgggggggaagccacagaggagaaagaatccacaggagggactgtataatgaattgcagaaagataaaatggcagaagcctacagtgaaatcgggatgaagggcgaaagacgcagagggaaggggcatgatggtttgtatcaagggctgtcaactgcgacaaaggacacgtatgatgcactgcatatgcaggctcttcccccgagg

SEQ ID NO: 101 - DNA, 2355 bp, synthetic construct
  source 1..2355: mol_type = other DNA
atggctcttcccgtgacggcactccttctgcccttggcactcctgctgcacgcggcaagaccagaggtgcagcttgtcgagtcagggggtggcctcgttcagccgggtggtagtctcaggctgagttgtgcagcttccgggttcactttctcagcatacgatatgtcctgggttaggcaagcaccggggaaacaacttgagtgggtttccgctatttctggcagtggtgggtccacgtattatgcggactcagtgaaaggccgcttcactattagccgggataattccaagaatacgctctacctgcaaatgaatagtctgagagcggaggatacggcggtttattattgtgcgtatgacggtgacagcacgggtagcctggacgaaatggcacactggggacaggggacccttgtgaccgtcagtagtactaccactcctgcaccacgacctcccacaccagcacctaccatagcttctcaacctctctctcttcggccagaagcttgtcgccctgcggcaggtggtgctgtacatacacgaggcctcgatttcgcttgcgatatatatatttgggcacctctcgctgggacgtgtggggtgcttcttctttccctcgtcatcacgttgtactgcaatcatcgcaatcgccggcgagtgtgcaaacggggtcgaaagaaactcctctacatttttaagcagccttttatgaggccggtacagacgacgcaggaggaggatgggtgttcttgtagattcccagaggaggaggaaggaggttgtgaacttcgggtcaaattctctcgctcagccgacgcaccggcctaccaacaggggcaaaatcaattgtataatgagttgaatctgggccgcagagaagagtacgacgtgctcgataagcgaaggggtagagatcctgaaatgggagggaaaccgcagcggaggaaaaacccccaagagggattgtacaatgagcttcagaaggataaaatggctgaagcttattccgagattggaatgaaaggagagcggcggcgcggtaaaggtcacgatggcttgtatcagggactgtccaccgcaacgaaggacacttacgacgcccttcatatgcaagcattgcctccacgaggatcaggcgctaccaatttttctcttctcaaacaggccggggacgttgaggaaaaccctggtccaggatccggagagggtagaggatccctcctgacctgcggtgacgtggaagagaacccaggtccaatggctctgccagtgactgccctccttctgcctcttgcactgcttcttcatgcggcaagaccagaggttcagctggttgaatctggtggaggattggtgcagccaggtggatctcttcggctcagttgcgccgcaagcggcttcacattttcatactacgcaatgtcttgggtgcggcaagcaccagggaaacaacgcgaatgggttagcgcaatcgagggcagtggtggtcggacatattacgcagacagcgttaagggaaggttcaccataagccgagacaattcaaagaatacactgtacttgcaaatgaattcccttagagcggaggatacagcggtatactactgtgtcaaagagaccgggtgggctgactttgactattggggccagggcaccctggttacggtatcaagcacacagcctcaagagacccagggtcccccggcgagaccgattactgtgcagcctactgaagcgtggccaagaacatctcagggacccagtacacggccggtagaggtccccgggggcagagcggtcgccgctatccttggactgggtttggtcttgggattgctcgggcctttggccatactgcttgcactctatctgctgagaagggatcagcgaaagcgaggtcggaaaaagttgctctacatctttaagcaaccattcatgcggcctgtacaaacaacccaggaggaagatggttgtagttgccgctttccagaagaggaagaaggtggctgcgaattgcgggtaaagttctcacgaagcgcagatgcgccggcttatcagcaaggtcaaaatcagttgtataacgagttgaatctgggaaggcgagaagagtatgatgttttggacaaacggagaggtcgagacccagaaatgggcggtaagccgcaaaggaggaaaaacccccaggaggggttgtataatgaattgcaaaaggacaaaatggctgaagcgtacagtgaaataggaatgaaaggggagagaaggcggggcaagggccatgacggactgtatcagggtctctccaccgcaactaaggatacatacgatgctttgcacatgcaggcacttccgccaaga

SEQ ID NO: 102 - protein, 1094 aa, synthetic construct
MALPVTALLLPLALLLHAARPEVQLVESGGGLVQPGGSLRLSCAASGFTFSTYDMSWVRQAPGKQREWVSAISGSGGTTYYADSVKGRFTISRDNSKNTLYLQMNSLRAEDTAVYYCVRGDDHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREGRGSLLTCGDVEENPGPMALPVTALLLPLALLLHAARPEVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRARAKRSGSGEGRGSLITCGDVEENPGPMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 103 - protein, 1096 aa, synthetic construct
MALPVTALLLPLALLLHAARPEVQLVESGGGLVQPGGSLRLSCAASGFTFSNYDMSWVRQAPGKQLEWVSAITGSGGSTYYADSVKGRFTISRDNSKNTLYLQMNSLRAEDTAVYYCAAGVDGDYWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREGRGSLLTCGDVEENPGPMALPVTALLLPLALLLHAARPEVQLVESGGGLVQPGGSLRLSCAASGFTFSTYAMSWVRQAPGKQREWVSAIEGSGGSTYYADSVKGRFTISRDNSKNTLYLQMNSLRAEDTAVYYCAVETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRARAKRSGSGEGRGSLITCGDVEENPGPMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 104 - protein, 1103 aa, synthetic construct
MALPVTALLLPLALLLHAARPEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPREGRGSLLTCGDVEENPGPMALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRARAKRSGSGEGRGSLITCGDVEENPGPMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 105 - protein, 1101 aa, synthetic construct
MALPVTALLLPLALLLHAARPEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGEGRGSLLTCGDVEENPGPMALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 106 - protein, 1144 aa, synthetic construct
MALPVTALLLPLALLLHAARPEVQLVESGGGLVQPGGSLRLSCAASGFTFSAYDMSWVRQAPGKQLEWVSAISGSGGSTYYADSVKGRFTISRDNSKNTLYLQMNSLRAEDTAVYYCAYDGDSTGSLDEMAHWGQGTLVTVSSTTTPAPRPPTPAPTIASQPLSLRPEACRPAAGGAVHTRGLDFACDIYIWAPLAGTCGVLLLSLVITLYCNHRNRRRVCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPGSGEGRGSLLTCGDVEENPGPMALPVTALLLPLALLLHAARPEVQLVESGGGLVQPGGSLRLSCAASGFTFSYYAMSWVRQAPGKQREWVSAIEGSGGRTYYADSVKGRFTISRDNSKNTLYLQMNSLRAEDTAVYYCVKETGWADFDYWGQGTLVTVSSTQPQETQGPPARPITVQPTEAWPRTSQGPSTRPVEVPGGRAVAAILGLGLVLGLLGPLAILLALYLLRRDQRKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPRGSGATNFSLLKQAGDVEENPGPGSGEGRGSLLTCGDVEENPGPMPRGWTALCLLSLLPSGFMSLDNNGTATPELPTQGTFSNVSTNVSYQETTTPSTLGSTSLHPVSQHGNEATTNITETTVKFTSTSVITSVYGNTNSSVQSQTSVISTVFTTPANVSTPETTLKPSLSPGNVSDLSTTSTSLATSPTKPYTSSSPILSDIKAEIKCSGIREVKLTQGICLEQNKTSSCAEFKKDRGEGLARVLCGEEQADADAGAQVCSLLLAQSEVRPQCLLLVLANRTEISSKLQLMKKHQSDLKKLGILDFTEQDVASHQSYSQKTLIALVTSGALLAVLGITGYFLMNRRSWSPTGERLELEP

SEQ ID NO: 107 - DNA, 3282 bp, synthetic construct
  source 1..3282: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagcacatatgatatgagctgggtccgccaggctccagggaagcagcgtgagtgggtctcagctatttccggtagtggtggtacaacatactacgcagactccgtgaagggccggttcaccatctccagagacaattccaagaacacgctgtatctgcaaatgaacagcctgagagccgaggacacggccgtatattactgtgtgcgcggcgacgaccattggggccagggaaccctcgtgacagttagcagcactacgactcctgcaccgcgaccgcccacaccagctcctaccatagcttcccaacctctctctcttcgcccagaagcgtgtcggcctgccgcgggtggcgcagtacacacgaggggcctggactttgcatgtgatatctacatctgggctccactcgcaggaacctgtggcgtattgcttctttccttggtgattactctctattgcaatcacagaaataggaggcgagtgtgtaaacgcgggcgcaagaagttgttgtacatattcaagcaaccctttatgagaccagtgcaaacaacacaagaagaggatggttgttcatgcagattccctgaggaggaagagggcggctgcgaacttagggtcaagttttcaaggtccgctgacgcaccggcataccagcaaggccaaaatcagctttataatgaactgaatctcgggcgccgagaagaatatgatgttcttgacaagaggcgcgggagggaccccgaaatggggggcaaaccccagcggaggaagaatccccaggaaggcctttacaatgaattgcagaaagacaaaatggctgaagcatacagtgaaataggaatgaagggggagaggcgccgcgggaagggacatgacggattgtatcagggattgagtactgcgacgaaagacacgtacgatgctcttcacatgcaagcgcttccaccgcgggagggcagaggaagtcttctaacatgcggtgacgtggaggagaatcccggccctatggcccttcccgtgacggctcttcttttgccactggcacttcttttgcacgcagcgcgacctgaggtgcagctggtggagtctgggggaggcttggtacagcctggggggtccctgagactctcctgtgcagcctctggattcacctttagcacgtatgccatgagctgggtccgccaggctccagggaagcagcgtgagtgggtctcagctattgaaggtagtggtggttctacatactacgcagactccgtgaagggccggttcaccatctccagagacaattccaagaacacgctgtatctgcaaatgaacagcctgagagccgaggacacggccgtatattactgtgccgtagagacaggatgggcggattttgactattggggccagggaaccctggtcaccgtttcatctacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctgcggagggaccagaggaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcccgcgccaaaaggtctggctccggtgagggcagaggaagtcttataacatgcggtgacgtggaggagaatcccggccctatgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatctacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcct

SEQ ID NO: 108 - DNA, 3288 bp, synthetic construct
  source 1..3288: mol_type = other DNA
atggcccttcccgttaccgccttactcttacctcttgctttattactgcacgcggcacggcctgaagtccaactcgtcgaaagcgggggaggcttggtacagcctggggggtccctgagactctcctgtgcagcctctggattcacctttagcaactatgacatgagctgggtccgccaggctccagggaagcagcttgagtgggtctcagctattaccggtagtggtggtagtacatactacgcagactccgtgaagggccggttcaccatctccagagacaattccaagaacacgctgtatctgcaaatgaacagcctgagagccgaggacacggccgtatattactgtgctgcaggcgtagatggcgactattggggccagggaaccctcgtgacagttagcagcactacgactcctgcaccgcgaccgcccacaccagctcctaccatagcttcccaacctctctctcttcgcccagaagcgtgtcggcctgccgcgggtggcgcagtacacacgaggggcctggactttgcatgtgatatctacatctgggctccactcgcaggaacctgtggcgtattgcttctttccttggtgattactctctattgcaatcacagaaataggaggcgagtgtgtaaacgcgggcgcaagaagttgttgtacatattcaagcaaccctttatgagaccagtgcaaacaacacaagaagaggatggttgttcatgcagattccctgaggaggaagagggcggctgcgaacttagggtcaagttttcaaggtccgctgacgcaccggcataccagcaaggccaaaatcagctttataatgaactgaatctcgggcgccgagaagaatatgatgttcttgacaagaggcgcgggagggaccccgaaatggggggcaaaccccagcggaggaagaatccccaggaaggcctttacaatgaattgcagaaagacaaaatggctgaagcatacagtgaaataggaatgaagggggagaggcgccgcgggaagggacatgacggattgtatcagggattgagtactgcgacgaaagacacgtacgatgctcttcacatgcaagcgcttccaccgcgggagggcagaggaagtcttctaacatgcggtgacgtggaggagaatcccggccctatggcccttcccgtgacggctcttcttttgccactggcacttcttttgcacgcagcgcgacctgaggtgcagctggtggagtctgggggaggcttggtacagcctggggggtccctgagactctcctgtgcagcctctggattcacctttagcacgtatgccatgagctgggtccgccaggctccagggaagcagcgtgagtgggtctcagctattgaaggtagtggtggttctacatactacgcagactccgtgaagggccggttcaccatctccagagacaattccaagaacacgctgtatctgcaaatgaacagcctgagagccgaggacacggccgtatattactgtgccgtagagacaggatgggcggattttgactattggggccagggaaccctggtcaccgtttcatctacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctgcggagggaccagaggaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcccgcgccaaaaggtctggctccggtgagggcagaggaagtcttataacatgcggtgacgtggaggagaatcccggccctatgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatctacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcct

SEQ ID NO: 109 - DNA, 3309 bp, synthetic construct
  source 1..3309: mol_type = other DNA
atggcccttcccgttaccgccttactcttacctcttgctttattactgcacgcggcacggcctgaggtgcagctggtggagtctgggggaggcttggtacagcctggggggtccctgagactctcctgtgcagcctctggattcacctttagcgcgtatgatatgagctgggtccgccaggctccagggaagcagcttgagtgggtctcagctattagcggtagtggtggtagcacatactacgcagactccgtgaagggccggttcaccatctccagagacaattccaagaacacgctgtatctgcaaatgaacagcctgagagccgaggacacggccgtatattactgtgcctacgatggtgattctacagggtctctggatgagatggcgcactggggccagggaaccctggtcaccgtttcatctactacgactcctgcaccgcgaccgcccacaccagctcctaccatagcttcccaacctctctctcttcgcccagaagcgtgtcggcctgccgcgggtggcgcagtacacacgaggggcctggactttgcatgtgatatctacatctgggctccactcgcaggaacctgtggcgtattgcttctttccttggtgattactctctattgcaatcacagaaataggaggcgagtgtgtaaacgcgggcgcaagaagttgttgtacatattcaagcaaccctttatgagaccagtgcaaacaacacaagaagaggatggttgttcatgcagattccctgaggaggaagagggcggctgcgaacttagggtcaagttttcaaggtccgctgacgcaccggcataccagcaaggccaaaatcagctttataatgaactgaatctcgggcgccgagaagaatatgatgttcttgacaagaggcgcgggagggaccccgaaatggggggcaaaccccagcggaggaagaatccccaggaaggcctttacaatgaattgcagaaagacaaaatggctgaagcatacagtgaaataggaatgaagggggagaggcgccgcgggaagggacatgacggattgtatcagggattgagtactgcgacgaaagacacgtacgatgctcttcacatgcaagcgcttccaccgcgggagggcagaggaagtcttctaacatgcggtgacgtggaggagaatcccggccctatggcccttcccgtgacggctcttcttttgccactggcacttcttttgcacgcagcgcgacctgaagtccaactcgtcgaaagcgggggaggcttggtacagcctggggggtccctgagactctcctgtgcagcctctggattcacctttagctactatgccatgagctgggtccgccaggctccagggaagcagcgtgagtgggtctcagctattgaaggtagtggtggtcggacatactacgcagactccgtgaagggccggttcaccatctccagagacaattccaagaacacgctgtatctgcaaatgaacagcctgagagccgaggacacggccgtatattactgtgttaaagagacaggatgggcggattttgactattggggccagggaaccctcgtgacagttagcagcacgcagccccaggagacccagggccccccggccaggcccatcactgtccagcccactgaagcctggcccagaacctcacagggaccctccacccggcccgtggaggtccccgggggccgtgcggttgccgccatcctgggcctgggcctggtgctggggctgctgggccccctggccatcctgctggccctgtacctgctgcggagggaccagaggaaacggggcagaaagaaactcctgtatatattcaaacaaccatttatgagaccagtacaaactactcaagaggaagatggctgtagctgccgatttccagaagaagaagaaggaggatgtgaactacgcgttaagttcagcaggagcgcagacgcccccgcgtaccagcagggccagaaccagctctataacgagctcaatctaggacgaagagaggagtacgatgttttggacaagcgccgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcgcccgcgccaaaaggtctggctccggtgagggcagaggaagtcttataacatgcggtgacgtggaggagaatcccggccctatgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatctacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcct

SEQ ID NO: 110 - DNA, 3303 bp, synthetic construct
  source 1..3303: mol_type = other DNA
atggcgctccccgtgaccgcgttgcttctcccgctggcactcctgttgcatgcggcacgaccggaagttcagctggtggagagcggaggaggcctcgtccaacctgggggctcacttaggttgtcttgcgcggcgagcggcttcaccttttcagcatatgatatgagttgggtaagacaagccccaggaaaacagctcgaatgggtatctgctataagtggatccggagggtcaacttactacgcggactctgttaaggggcgctttacaattagccgcgacaacagtaagaacactttgtaccttcagatgaactcacttcgggccgaggacacagctgtatattactgtgcatatgatggggattctactggctctctggatgagatggcccactggggtcaaggcacactcgtgacggtttcctcaaccaccacgccagcgcctagaccccctactccggcccctacaatagcaagtcaacccctctctctgagacctgaagcctgccgacccgcagcgggtggagctgtccacacccgcggcctggatttcgcatgtgacatatacatatgggcgcccctggcaggtacctgcggtgttctgttgcttagccttgtaattacgctttactgtaaccacagaaatcgacggagggtctgtaagcgcggtcggaagaagctcttgtacattttcaagcagccttttatgaggccggtccaaacgacacaagaggaagacggatgctcatgtaggtttccagaggaagaggaaggaggctgtgaacttcgggtcaaatttagtagatctgcggatgcaccggcatatcaacaagggcaaaatcaactgtacaacgagctcaatctgggtaggcgagaagaatatgacgtacttgataagcggcggggacgggatcctgaaatgggtggtaaaccacagcgcaggaaaaacccgcaggaggggctttataacgagcttcaaaaagacaaaatggcggaggcctactccgaaataggcatgaagggcgaaagacgacgcggtaaggggcacgatggcttgtaccaagggttgtccaccgcgactaaagacacatacgacgctcttcatatgcaggcgttgccgccgaggggttcaggcgaaggccggggcagtcttttgacatgcggcgatgttgaggagaaccctggacccatggcgcttcctgtaactgcactgcttctcccactcgcattgcttctgcatgcagcccgccccgaagttcaactcgtagaatccgggggaggcttggtccagccaggtggatccctccgactctcatgtgccgcaagtgggtttacgttcagttattacgcaatgtcatgggtgagacaggcaccagggaaacagagagaatgggtaagcgcaatcgagggatccggagggcggacctactacgccgactccgtcaagggacgcttcacgattagcagggacaacagtaagaacaccttgtacctgcaaatgaattcacttcgagctgaagacactgccgtttattattgcgtcaaggaaacgggctgggccgattttgattattggggacagggaacattggtcacggtgtcatcaacccagccgcaagagactcaagggccgcccgccaggccgataacggtacagcccactgaggcgtggccacgaacctctcagggcccatccactaggcccgtcgaagtacctggggggagagccgtcgctgccatcctggggctcggtctggttttgggcttgttgggtccgcttgcaattttgttggccttgtaccttctgcggagagaccagcggaagcggggtaggaaaaaacttctgtatatattcaagcaacccttcatgcgcccggtgcagaccacccaagaagaggatgggtgcagctgcaggtttccagaggaggaggaaggcggctgcgagttgcgggtaaagttttcaaggtcagcagatgctccggcctaccagcaaggacagaaccaattgtacaatgagctgaatttgggtcgccgagaagaatatgacgtcttggataagcgcagaggacgggacccagaaatgggggggaagccacagaggagaaagaatccacaggagggactgtataatgaattgcagaaagataaaatggcagaagcctacagtgaaatcgggatgaagggcgaaagacgcagagggaaggggcatgatggtttgtatcaagggctgtcaactgcgacaaaggacacgtatgatgcactgcatatgcaggctcttcccccgaggggctcaggtgcaaccaatttcagccttctgaaacaggctggggatgtggaagaaaatcccggcccgatgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatctacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcct

SEQ ID NO: 111 - DNA, 3432 bp, synthetic construct
  source 1..3432: mol_type = other DNA
atggctcttcccgtgacggcactccttctgcccttggcactcctgctgcacgcggcaagaccagaggtgcagcttgtcgagtcagggggtggcctcgttcagccgggtggtagtctcaggctgagttgtgcagcttccgggttcactttctcagcatacgatatgtcctgggttaggcaagcaccggggaaacaacttgagtgggtttccgctatttctggcagtggtgggtccacgtattatgcggactcagtgaaaggccgcttcactattagccgggataattccaagaatacgctctacctgcaaatgaatagtctgagagcggaggatacggcggtttattattgtgcgtatgacggtgacagcacgggtagcctggacgaaatggcacactggggacaggggacccttgtgaccgtcagtagtactaccactcctgcaccacgacctcccacaccagcacctaccatagcttctcaacctctctctcttcggccagaagcttgtcgccctgcggcaggtggtgctgtacatacacgaggcctcgatttcgcttgcgatatatatatttgggcacctctcgctgggacgtgtggggtgcttcttctttccctcgtcatcacgttgtactgcaatcatcgcaatcgccggcgagtgtgcaaacggggtcgaaagaaactcctctacatttttaagcagccttttatgaggccggtacagacgacgcaggaggaggatgggtgttcttgtagattcccagaggaggaggaaggaggttgtgaacttcgggtcaaattctctcgctcagccgacgcaccggcctaccaacaggggcaaaatcaattgtataatgagttgaatctgggccgcagagaagagtacgacgtgctcgataagcgaaggggtagagatcctgaaatgggagggaaaccgcagcggaggaaaaacccccaagagggattgtacaatgagcttcagaaggataaaatggctgaagcttattccgagattggaatgaaaggagagcggcggcgcggtaaaggtcacgatggcttgtatcagggactgtccaccgcaacgaaggacacttacgacgcccttcatatgcaagcattgcctccacgaggatcaggcgctaccaatttttctcttctcaaacaggccggggacgttgaggaaaaccctggtccaggatccggagagggtagaggatccctcctgacctgcggtgacgtggaagagaacccaggtccaatggctctgccagtgactgccctccttctgcctcttgcactgcttcttcatgcggcaagaccagaggttcagctggttgaatctggtggaggattggtgcagccaggtggatctcttcggctcagttgcgccgcaagcggcttcacattttcatactacgcaatgtcttgggtgcggcaagcaccagggaaacaacgcgaatgggttagcgcaatcgagggcagtggtggtcggacatattacgcagacagcgttaagggaaggttcaccataagccgagacaattcaaagaatacactgtacttgcaaatgaattcccttagagcggaggatacagcggtatactactgtgtcaaagagaccgggtgggctgactttgactattggggccagggcaccctggttacggtatcaagcacacagcctcaagagacccagggtcccccggcgagaccgattactgtgcagcctactgaagcgtggccaagaacatctcagggacccagtacacggccggtagaggtccccgggggcagagcggtcgccgctatccttggactgggtttggtcttgggattgctcgggcctttggccatactgcttgcactctatctgctgagaagggatcagcgaaagcgaggtcggaaaaagttgctctacatctttaagcaaccattcatgcggcctgtacaaacaacccaggaggaagatggttgtagttgccgctttccagaagaggaagaaggtggctgcgaattgcgggtaaagttctcacgaagcgcagatgcgccggcttatcagcaaggtcaaaatcagttgtataacgagttgaatctgggaaggcgagaagagtatgatgttttggacaaacggagaggtcgagacccagaaatgggcggtaagccgcaaaggaggaaaaacccccaggaggggttgtataatgaattgcaaaaggacaaaatggctgaagcgtacagtgaaataggaatgaaaggggagagaaggcggggcaagggccatgacggactgtatcagggtctctccaccgcaactaaggatacatacgatgctttgcacatgcaggcacttccgccaagaggctcaggagctacaaatttctcactcttgaagcaagcgggagatgtcgaagaaaatcctggtccgggttcaggcgagggacgcggatcattgctcacctgtggggatgtggaagagaaccccggtccgatgcctcgcggctggacagccctgtgcctgctgtctctgctgccatccggcttcatgagcctggataataacggcacagccaccccagagctgcctacacagggcaccttcagcaatgtgtccacaaacgtgagctatcaggagaccacaaccccttctaccctgggatctacaagcctgcaccccgtgtctcagcacggcaacgaagccaccaccaacatcaccgagaccacagtgaagtttacctccacctctgtgattacctctgtgtacggaaatacaaactccagcgtgcagtctcagacatctgtgatctccacagtgtttacaacacctgccaatgtgtccaccccagagacaaccctgaagcccagcctgtctcctggaaatgtgtccgatctgtctaccacctccaccagcctggccacctctcccaccaagccctatacctcctcttctcccatcctgagcgatatcaaagccgagatcaaatgcagcgggattcgggaagtgaaactgacacagggcatctgcctggaacagaataagacatccagctgcgccgagtttaagaaagatagaggagagggactggccagggtgctgtgtggcgaagagcaggccgacgccgatgccggcgcccaggtgtgttccctgctgctggcccagtctgaggtgcgcccccagtgcctgctgctggtgctggccaatcggacagaaattagcagcaagctgcagctgatgaaaaaacaccagagcgatctgaaaaagctgggcatcctggactttaccgagcaggacgtggcctctcaccagagctacagccagaaaacactgatcgccctggtgaccagcggagccctgctggccgtgctgggcatcaccggatatttcctgatgaataggcgcagctggagccccaccggcgagcggctggagctggagcct

(19) United States
(12) Patent Application Publication
(10) Pub. No.: US 2026/0224702 A1
(43) Pub. Date: Aug. 6, 2026

(54) CLL1 AND/OR CD33 BINDING MOLECULES

(71) Applicant: CODING BIO LIMITED, Oxford (GB)

(72) Inventors: Simon BORNSCHEIN, Oxford (GB); Youlia LAMPI, Oxford (GB); Rui Tiago De Lima RODRIGUES, Oxford (GB); Alice Louise GODSON, Oxford (GB)

(21) Appl. No.: 19/154,238

(22) PCT Filed: Feb. 12, 2024
(86) PCT No.: PCT/GB2024/050373

§ 371 Date: Aug. 6, 2025

(30) Foreign Application Priority Data
Feb. 10, 2023 (GB) 2301949.0

(51) Int. Cl. A61K 40/33; A61K 38/00; A61K 40/11; A61K 40/41; A61P 35/00; C07K 14/705; C07K 16/28; C12N 5/0783

(52) U.S. Cl. CPC A61K 40/33; A61K 40/11; A61K 40/41; A61P 35/00; C07K 14/7051; C07K 14/70517; C07K 14/70578; C07K 16/2803; C07K 16/2809; C07K 16/2851; C12N 5/0636; A61K 38/00; C07K 2317/569; C07K 2319/02; C07K 2319/03; C12N 2510/00

(57) ABSTRACT

The present disclosure relates to a novel class of C-type lectin-like molecule 1 (CLL1) binding molecules and/or sialic acid binding Ig-like lectin 3 (SIGLEC-3 or CD33) binding molecules, in particular to anti-CLL1 and/or anti-CD33 antibodies and antigen binding fragments. This disclosure also relates to the synthetic molecules comprising the anti-CLL1 and/or anti-CD33 antibodies and antigen binding fragments, such as chimeric antigen receptors (CARs), bispecific T-Cell engagers (BiTEs) and antibody-drug conjugates (ADCs), and use of the anti-CLL1 and/or anti-CD33 antibodies and antigen binding fragments, or synthetic molecules comprising them, in the treatment of disease.

### FIELD OF THE INVENTION

The present invention relates to C-type lectin-like molecule 1 (CLL1) and sialic acid binding Ig-like lectin 3 (SIGLEC-3 or CD33) binding molecules, in particular to anti-CLL1 and/or anti-CD33 antibodies and antigen binding fragments. The present invention further relates to recombinant molecules comprising said CLL1/CD33 binding molecules, such as, chimeric antigen receptors (CARs), BiTEs and ADCs. Another object of the present invention is an engineered immune cell, suitably, a T cell comprising said CAR. Uses thereof in therapeutic methods of said antibody, said recombinant molecule and said engineered immune cell are further described.

### BACKGROUND OF THE INVENTION

The C-type lectin-like molecule 1 (CLL1) glycoprotein (also known as CLEC12A, DCAL2, MICL, KLRL1) is a type II transmembrane protein comprising a single C-type lectin-like domain, a transmembrane domain, a stalk region, and a cytoplasmic tail containing an immunoreceptor tyrosine-based inhibitory motif (ITIM) (https://www.uniprot.org/uniprotkb/Q5QGZ9/entry; Marshall et al. 2006. Human CLL1 is differentially glycosylated and is down-regulated following cellular activation. Eur. J. Immunol., 36, 2159-2169).

CLL1 functions as a cell surface receptor and is expressed on myeloid lineage cells. The cytoplasmic ITIM motif is a conserved sequence of amino acids, commonly found in a number of immune-function related receptors, which contains a tyrosine residue that may be phosphorylated and thereby effect a signalling cascade which is thought to be important for regulating cellular maturation and activation.

CD33 is a protein that is expressed on the surface of myeloid cells, including acute myeloid leukaemia (AML) cells, and plays a role in regulating the growth and differentiation of these cells. CD33 is a member of the sialic acid-binding immunoglobulin-like lectin (Siglec) family of receptors. One of the main functions of CD33 is to act as a negative regulator of myeloid cell activation.

Importantly, while CLL1 and CD33 are expressed on myeloid lineage cells, these receptors show limited expression in other cell lineages and tissues and are not expressed on haematopoietic stem cells (HSCs). Furthermore, CLL1 and CD33 have been found to be expressed in more than 80% of AML patients. Therefore, CLL1 and CD33 represent good targets for immunotherapy as they appear to be attractive proteins to discriminate between AML and non-AML cells.

AML typically affects middle aged and older adults and represents approximately 1% of new cancer cases every year in the USA (Cancer Stat Facts: Leukemia-Acute Myeloid Leukemia (AML). Available online: https://seer.cancer.gov/statfacts/html/amyl.html (accessed on 5 Sep. 2022)). Historically, AML has been challenging to treat and is associated with a high mortality rate. For example, in the USA, AML has a 5-year relative survival rate of 30.5% (Cancer Stat Facts: Leukemia-Acute Myeloid Leukemia (AML). Available online: https://seer.cancer.gov/statfacts/html/amyl.html (accessed on 5 Sep. 2022)). One reason for this difficulty is that AML has many antigens in common with haematopoietic stem cells (HSCs). Hence, in most instances of AML new therapeutic methods, such as immunotherapy, it has not been possible to specifically target cancerous cells.

CLL1 is also implicated with other cancers and diseases including but not limited to: AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN), or combinations thereof. CD33 is implicated in AML and some types of lymphoma. Several studies have demonstrated that targeting CD33 with monoclonal antibodies or small molecule inhibitors can effectively kill AML cells in vitro and in vivo (Luger S M, Fatula J A, Heerema N A, et al. Phase 3 study of gemtuzumab ozogamicin in older adults with newly diagnosed acute myeloid leukemia. N Engl J Med. 2019; 380 (4): 328-338. doi: 10.1056/NEJMoa1806772).

CLL1 may additionally play a role in inflammatory or autoimmune diseases such as rheumatoid arthritis, psoriasis, allergies, asthma, Crohn's disease, IBD, IBS, fibromyalga, mastocytosis, and Celiac disease.

Therapeutic treatments that make use of the immune system or immune cells are known as immunotherapy treatments. In some cases, immunotherapy treatment relies on antibodies to an antigen on a target cell triggering or promoting cell death or inhibiting cell-division. Antigens may be targeted using antibody or antigen binding fragments, such as a single chain variable fragment (scFv). Examples of antibodies that target CLL1 have been previously described e.g. U.S. Pat. No. 9,163,090 B2.

The antibody or antigen binding domain can be linked or conjugated to a synthetic molecule, such as a synthetic molecule that binds to a T-cell antigen, suitably via a linker, such as a flexible peptide linker to form a bi-specific T-cell engager (BiTE), or a dual-affinity retargeting antibody (DART) or other bi-specific, tri-specific or multi-specific T-cell engagers.

Alternatively, such antibodies or antigen binding fragments may be linked or conjugated with a synthetic molecule such as a label, cytotoxic agent (antibody-drug conjugates or ADCs), or therapeutic isotope.

A specific type of immunotherapy is chimeric antigen receptor (CAR) T-cell therapy. CAR T-cell therapy consists of obtaining T-cells from a patient and incorporating an engineered receptor to recognise an antigen found on the cancerous cells. The resulting CAR T-cells are then incorporated back into the patient, where they may then act to mount an immune response against the antigen i.e. against the cancerous cells and kill the cancerous cells.

In this case, one or more antibodies or antigen binding fragments can be linked or conjugated with a synthetic molecule comprising other protein domains, such as hinge domains, transmembrane domains, co-stimulation domains and activation domains to form a CAR that is capable of effecting an immune response towards the antigen-containing cell. This CAR is then expressed in a T-cell.

Examples of CARs that target CLL1 have been previously described e.g. U.S. Pat. No. 10,568,947 B2 and U.S. Pat. No. 10,597,456 B2 and Zhang et al. Clin Cancer Res. 2021; 27 (13): 3549-3555,

Two recent reports have claimed success of CLL1-based CAR T cells in patients with secondary AML (Zhang et al. Front Oncol. 2020; 10:685; and Fang et al. Blood. 2018; 132:901), thereby highlighting potential for use in AML and other cancers.

The nature of the binding between the CAR and the target antigen, and the associated activation of the T-cell as a result of this binding via other functional domains, is critical to the effectiveness of the CAR T-cell therapy.

Producing CARs that demonstrate high activation from low levels of CLL1 is important due to the known variable level of CLL1 expression in cells, and the potential for antigen escape during immunotherapy treatment.

Previous anti-CLL1 CARs have shown varying levels of effectiveness at eliciting an immune response and killing cancer cells. The reasons for this remain unknown, but it may be related to a low binding affinity of the antibody or antibody derivative portion of the CAR, which results in poor binding to CLL1 and weaker activation of the immune response. Alternatively, this may be due to the lack of optimisation or co-optimisation of the remaining functional domains of the CAR, which also affect T-cell activation.

CD33 CAR-T cell constructs have, similarly to CLL1, shown clinical activity. However, for both targets, cancer cells commonly evolve to lose CD33 or CLL1 expression, and hence become resistant to the targeted therapy over time, so-called “antigen escape”.

Commonly, for CARS for example, the combination of two scFvs into a single CAR is considered for such an application. However, two or more scFvs present on the same antibody has the potential to mismatch VH/VL sequences due to close proximity. VH/VL mismatched binders can have a detrimental effect on binding of each scFv and/or potentially unwanted off-target activity. Besides having potential unwanted off-target binding activity, in the context of CAR constructs this can also induce tonic activation of the CAR.

Therefore, there exists a need for novel and improved antibodies or antigen binding fragments, suitable for treating CLL1/CD33-associated diseases and disorders.

### SUMMARY OF THE INVENTION

In a first aspect, the invention provides an antigen binding molecule that binds CLL1 and/or CD33 (i.e. that binds CLL1, or CD33, or both CLL1 and CD33); wherein the antigen binding molecule comprises an antigen binding domain selected from the group consisting of:
Antigen binding domain 1, comprising a single variable domain on a heavy chain comprising,
   (i) a CDR1 of SEQ ID NO:8,
   (ii) a CDR2 of SEQ ID NO:11, and
   (iii) a CDR3 of SEQ ID NO:14;
and,
Antigen binding domain 2, comprising a single variable domain on a heavy chain comprising,
   (i) a CDR1 of SEQ ID NO:9,
   (ii) a CDR2 of SEQ ID NO: 12, and
   (iii) a CDR3 of SEQ ID NO:15;
and,
Antigen binding domain 3, comprising a single variable domain on a heavy chain comprising,
   (i) a CDR1 of SEQ ID NO:10,
   (ii) a CDR2 of SEQ ID NO:13, and
   (iii) a CDR3 of SEQ ID NO:16;
and,
Antigen binding domain 4, comprising a single variable domain on a heavy chain comprising,
   (i) a CDR1 of SEQ ID NO:19,
   (ii) a CDR2 of SEQ ID NO:21, and
   (iii) a CDR3 of SEQ ID NO:23;
and,
Antigen binding domain 5, comprising a single variable domain on a heavy chain comprising,
   (i) a CDR1 of SEQ ID NO:20,
   (ii) a CDR2 of SEQ ID NO:22, and
   (iii) a CDR3 of SEQ ID NO:24.

In embodiments, Antibody binding domains 1, 2 and 3 bind (suitably specifically bind) CLL1 and Antibody binding domains 4 and 5 bind (suitably specifically bind) CD33.

In embodiments, the antigen binding molecule binds to CLL1 and CD33. Suitably, the antigen binding molecule comprises one of antigen binding domain 1 to 3 (i.e. 1, 2 or 3) and one of antigen binding domain 4 to 5 (i.e. 4 or 5).

In embodiments, the antigen binding molecule comprises:
a. Antigen binding domain 1 and antigen binding domain 4; or
b. Antigen binding domain 2 and antigen binding domain 4; or
c. Antigen binding domain 3 and antigen binding domain 5.

In embodiments, the single variable domain on a heavy chain of any one of antigen binding domain 1 to 5 (i.e. 1, 2, 3, 4 or 5) is a VHH. Suitably, the VHH is selected from the group consisting of: SEQ ID NOs 5 to 7 (i.e. 5, 6 or 7) and SEQ ID NOs 17 to 18 (i.e. 17 or 18).

In embodiments, the antigen binding molecule is a chimeric antigen receptor further comprises a hinge domain (region), a transmembrane domain (region) and an intracellular T-cell receptor signaling domain (region). Suitably, the hinge region and the transmembrane region is from CD8A or TNFRSF4. Suitably, the intracellular T-cell receptor signaling domain is from CD247 (CD3-zeta). In embodiments, the antigen binding molecule further comprises an intracellular domain of a costimulatory protein receptor. Suitably, the antigen binding molecule further comprises an intracellular domain of a costimulatory protein receptor is from CD137.

In embodiments, the antigen binding molecule is a bi-specific T-cell engager (BiTE) comprising a T-cell antigen binding domain. Suitably, the T-cell antigen binding domain comprises an antibody fragment that specifically binds CD3.

In embodiments, the antigen binding molecule is an antibody-drug conjugate (ADC) comprising a cytotoxic drug or a therapeutic radioisotope.

In second aspect, the invention provides a chimeric antigen receptor that binds CLL1 and/or CD33 comprising one or more antigen binding domains selected from the group consisting of:
Antigen binding domain 1, comprising a single variable domain on a heavy chain comprising,
   (i) a CDR1 of SEQ ID NO:8,
   (ii) a CDR2 of SEQ ID NO:11, and
   (iii) a CDR3 of SEQ ID NO:14;
and,
Antigen binding domain 2, comprising a single variable domain on a heavy chain comprising,
   (i) a CDR1 of SEQ ID NO:9,
   (ii) a CDR2 of SEQ ID NO:12, and
   (iii) a CDR3 of SEQ ID NO:15;
and,
Antigen binding domain 3, comprising a single variable domain on a heavy chain comprising,
   (i) a CDR1 of SEQ ID NO:10,
   (ii) a CDR2 of SEQ ID NO:13, and
   (iii) a CDR3 of SEQ ID NO:16;
and,
Antigen binding domain 4, comprising a single variable domain on a heavy chain comprising,
   (i) a CDR1 of SEQ ID NO:19,
   (ii) a CDR2 of SEQ ID NO:21, and
   (iii) a CDR3 of SEQ ID NO:23;
and,
Antigen binding domain 5, comprising a single variable domain on a heavy chain comprising,
   (i) a CDR1 of SEQ ID NO:20,
   (ii) a CDR2 of SEQ ID NO:22, and
   (iii) a CDR3 of SEQ ID NO:24;
b) A transmembrane domain; and
c) An intracellular signaling domain.

In embodiments, a CAR comprising Antibody binding domains 1, 2 and 3 binds (suitably specifically binds) CLL1, and wherein a CAR comprising Antibody binding domains 4 and 5 binds (suitably specifically binds) CD33, and wherein a CAR comprising one of Antibody binding domains 1, 2 and 3 binds and one of Antibody binding domains 4 and 5 binds (suitably specifically binds) CLL1 and CD33.

In embodiments, the CAR further comprises a signal peptide.

In embodiments, the CAR has a structure comprising: a signal peptide domain; an antigen binding domain; a hinge domain; a transmembrane domain; and an intracellular signalling domain. Suitably, the CAR has a structure with the order of domains from the N-terminus to the C-terminus comprising: a signal peptide domain linked to an antigen binding domain linked to a hinge domain linked to a transmembrane domain linked to an intracellular signalling domain. Suitably, each domain of the CAR is linked directly to the next domain (i.e. with no additional amino acid residues between the domains).

In embodiments of the CAR of the second aspect, the transmembrane domain is from CD8A or TNFRSF4. Suitably, the hinge domain and the transmembrane domain is from CD8A or TNFRSF4. In embodiments, the intracellular signaling domain is from CD247 (CD3-zeta).

In embodiments, the CAR further comprises an intracellular signaling domain of a costimulatory protein receptor. Suitably, the intracellular signaling domain of a costimulatory protein receptor is positioned between, and linked at the N-terminus to the transmembrane domain and at the C-terminus to the intracellular signalling domain. Suitably, the intracellular signaling domain of a costimulatory protein receptor is from CD137.

In embodiments of the chimeric antigen receptor of the second aspect, the single variable domain on a heavy chain of any one of antigen binding domain 1, 2, 3, 4 or 5 is a VHH. Suitably, the VHH is selected from the group consisting of: SEQ ID NOs 5 to 7 (i.e. 5, 6 or 7) and SEQ ID NOs 17 to 18 (i.e. 17 or 18).

In embodiments, the CAR comprises:
a. a CD8A extracellular signal peptide (e.g. SEQ ID NO:39); one or more antigen binding domains of the antigen binding molecule of the first aspect (e.g. SEQ ID NOS: 5, 6 or 7 and SEQ ID NOS: 17 or 18); a human CD8A hinge/transmembrane domain (e.g. SEQ ID NO:34), a human CD137 co-stimulatory domain (e.g. SEQ ID NO:33); and a human CD247 (CD3-zeta) intracellular signaling domain (e.g. SEQ ID NO:32); or
b. a CD8A extracellular signal peptide (e.g. SEQ ID NO:39); one or more antigen binding domains of the antigen binding molecule of the first aspect (e.g. SEQ ID NOS: 5, 6 or 7 and SEQ ID NOS: 17 or 18); a human TNFRSF4 hinge/transmembrane domain (e.g. SEQ ID NO:35); a human CD137 co-stimulatory domain (e.g. SEQ ID NO:33); a human CD137 co-stimulatory domain (e.g. SEQ ID NO:33); and a human CD247 (CD3-zeta) intracellular signaling domain (e.g. SEQ ID NO:32). In embodiments, the CAR comprises 1, 2, 3 or 4 antigen binding domains of the antigen binding molecule of the first aspect (e.g. SEQ ID NOS: 5, 6 or 7 and SEQ ID NOS: 17 or 18). Suitably, the CAR comprises 1 or 2 antigen binding domains of the antigen binding molecule of the first aspect (e.g. SEQ ID NOS: 5, 6 or 7 and SEQ ID NOS: 17 or 18). Suitably, the CAR comprises 2 antigen binding domains of the antigen binding molecule of the first aspect (e.g. SEQ ID NOS: 5, 6 or 7 and SEQ ID NOS: 17 or 18). In embodiments, the chimeric antigen receptor binds CLL1 and CD33. Suitably, the chimeric antigen receptor comprises one of antigen binding domain 1 to 3 (i.e. 1, 2 or 3) and one of antigen binding domain 4 to 5 (i.e. 4 or 5). Suitably, the chimeric antigen receptor of the second aspect comprises:
a. Antigen binding domain 1 and antigen binding domain 4; or
b. Antigen binding domain 2 and antigen binding domain 4; or
c. Antigen binding domain 3 and antigen binding domain 5.

In embodiments, the CAR comprises two antigen binding domains, the antigen binding domains being joined by at least one linker. This is referred to in the art, and is defined herein, as a “tandem” CAR. Suitably, the linker has a sequence selected from the group consisting of: SEQ ID NOS: 36 to 38 (i.e. 36, 37 or 38).

In embodiments, the CAR is a tandem CAR, comprising:
a. a CD8A extracellular signal peptide (e.g. SEQ ID NO: 39); a first antigen binding domain of the antigen binding molecule of the first aspect (e.g. SEQ ID NOS: 5, 6 or 7); a linker (e.g. SEQ ID NOS: 36, 37 or 38); a second antigen binding domain of the antigen binding molecule of the first aspect (e.g. SEQ ID NOS: 17 or 18); a human CD8A hinge/transmembrane domain (e.g. SEQ ID NO:34), a human CD137 co-stimulatory domain (e.g. SEQ ID NO: 33); and a human CD247 (CD3-zeta) intracellular signalling domain (e.g. SEQ ID NO: 32); or
b. a CD8A extracellular signal peptide (e.g. SEQ ID NO:39); a first antigen binding domain of the antigen binding molecule of the first aspect (e.g. SEQ ID NOS: 17 or 18); a linker (e.g. SEQ ID NOS: 36, 37 or 38); a second antigen binding domain of the antigen binding molecule of the first aspect (e.g. SEQ ID NOS: 5, 6 or 7); a human CD8A hinge/transmembrane domain (e.g. SEQ ID NO:34), a human CD137 co-stimulatory domain (e.g. SEQ ID NO: 33); and a human CD247 (CD3-zeta) intracellular signalling domain (e.g. SEQ ID NO: 32); or
c. a CD8A extracellular signal peptide (e.g. SEQ ID NO: 39); a first antigen binding domain of the antigen binding molecule of the first aspect (e.g. SEQ ID NOS: 5, 6 or 7); a linker (e.g. SEQ ID NOS: 36, 37 or 38); a second antigen binding domain of the antigen binding molecule of the first aspect (e.g. SEQ ID NOS: 17 or 18); a human TNFRSF4 hinge/transmembrane domain (e.g. SEQ ID NO: 35); a human CD137 co-stimulatory domain (e.g. SEQ ID NO: 33); a human CD137 co-stimulatory domain (e.g. SEQ ID NO: 33); and a human CD247 (CD3-zeta) intracellular signalling domain (e.g. SEQ ID NO: 32); or
d. a CD8A extracellular signal peptide (e.g. SEQ ID NO:39); a first antigen binding domain of the antigen binding molecule of the first aspect (e.g. SEQ ID NOS: 17 or 18); a linker (e.g. SEQ ID NOS: 36 to 38); a second antigen binding domain of the antigen binding molecule of the first aspect (e.g. SEQ ID NOS: 5, 6 or 7); a human TNFRSF4 hinge/transmembrane domain (e.g. SEQ ID NO: 35); a human CD137 co-stimulatory domain (e.g. SEQ ID NO: 33); and a human CD247 (CD3-zeta) intracellular signalling domain (e.g. SEQ ID NO: 32).

In embodiments, the CAR is encoded by a nucleic acid sequence comprising two or more CAR sequences, joined by a sequence encoding a self-cleaving linker (self-cleaving peptide linker). This is referred to in the art, and is defined herein, as a “parallel” CAR Suitably, the self-cleaving linker has a sequence selected from the group consisting of: SEQ ID NO 43, 88 and 89.

In embodiments, the CAR is a parallel CAR encoded by a nucleic acid sequence comprising two or more CAR sequences comprising:
a. a CD8A extracellular signal peptide (e.g. SEQ ID NO: 39); a first antigen binding domain of the antigen binding molecule of the first aspect (e.g. SEQ ID NOS: 5, 6 or 7); a human CD8A hinge/transmembrane domain (e.g. SEQ ID NO: 34), a human CD137 co-stimulatory domain (e.g. SEQ ID NO: 33); and a human CD247 (CD3-zeta) intracellular signalling domain (e.g. SEQ ID NO: 32); a self-cleaving linker (e.g. SEQ ID NO: 43); a CD8A extracellular signal peptide (e.g. SEQ ID NO: 39); a second antigen binding domain of the antigen binding molecule of the first aspect (e.g. SEQ ID NOS: 17 or 18); a human TNFRSF4 hinge/transmembrane domain (e.g. SEQ ID NO: 35); a human CD137 co-stimulatory domain (e.g. SEQ ID NO: 33); and a human CD247 (CD3-zeta) intracellular signalling domain (e.g. SEQ ID NO: 32).

In embodiments, the CAR has a sequence selected from the group consisting of: SEQ ID NOS: 25 to 31; SEQ ID NOS: 40 to 42, 93 and 94 and SEQ ID NOS: 44 to 46, 95, 96 and 102 to 106.

In embodiments, the nucleic acid sequence is selected from the group consisting of: SEQ ID NOS: 83 to 85; 100 to 101 and 107 to 111

In a third aspect, the invention provides a recombinant T cell comprising the chimeric antigen receptor of the second aspect.

In a fourth aspect, the invention provides a nucleic acid having a sequence encoding the antigen binding molecule of the first aspect of the invention, or the CAR of the second aspect of the invention. Suitably, the nucleic acid comprises a nucleic acid sequence selected from the group consisting of: SEQ ID NOS 73 to 85; SEQ ID NOS 97 to 101 and SEQ ID NOS 107 to 111. Suitably the nucleic acid is a vector.

In a fifth aspect, the invention provides a vector comprising the nucleic acid of the fourth aspect.

In a sixth aspect, the invention provides a pharmaceutical composition comprising:
a. the antigen binding molecule of the first aspect of the invention; or
b. the CAR of the second aspect of the invention; or
c. the recombinant T cell of the third aspect of the invention; or
d. the nucleic acid of the fourth aspect of the invention; or
e. the vector of the fifth aspect of the invention;
   and a pharmaceutically acceptable carrier.

In a seventh aspect, the invention provides the antigen binding molecule of the first aspect, or the chimeric antigen receptor of the second aspect, or the recombinant T cell of the third aspect, or the nucleic acid of the fourth aspect, or the vector of the fifth aspect of the invention, or the pharmaceutical composition of the sixth aspect, for use as a medicament.

In an eighth aspect, the invention provides the antigen binding molecule of the first aspect, or the chimeric antigen receptor of the second aspect, or the recombinant T cell of the third aspect, or the nucleic acid of the fourth aspect, or the vector of the fifth aspect of the invention, or the pharmaceutical composition of the sixth aspect, for use in killing or inhibiting the growth of cells expressing CLL1 and/or CD33.

In a ninth aspect, the invention provides the antigen binding molecule of the first aspect, or the chimeric antigen receptor of the second aspect, or the recombinant T cell of the third aspect, or the nucleic acid of the fourth aspect, or the vector of the fifth aspect of the invention, or the pharmaceutical composition of the sixth aspect, for use in the treatment of a disease or disorder selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

In a tenth aspect, the invention provides a method of treatment of a disorder or disease, wherein said method comprises administering to a patient in need thereof, the invention provides the antigen binding molecule of the first aspect, or the chimeric antigen receptor of the second aspect, or the recombinant T cell of the third aspect, or the nucleic acid of the fourth aspect, or the vector of the fifth aspect of the invention, or the pharmaceutical composition of the sixth aspect.

In embodiments, the disorder or disease is selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

Also described is a bispecific T-cell engager that binds CLL1 and/or CD33 comprising one or more antigen binding domains selected from the group consisting of:
Antigen binding domain 1, comprising a single variable domain on a heavy chain comprising,
   (i) a CDR1 of SEQ ID NO:8,
   (ii) a CDR2 of SEQ ID NO:11, and
   (iii) a CDR3 of SEQ ID NO:14;
and,
Antigen binding domain 2, comprising a single variable domain on a heavy chain comprising,
   (i) a CDR1 of SEQ ID NO:9,
   (ii) a CDR2 of SEQ ID NO:12, and
   (iii) a CDR3 of SEQ ID NO:15;
and,
Antigen binding domain 3, comprising a single variable domain on a heavy chain comprising,
   (i) a CDR1 of SEQ ID NO:10,
   (ii) a CDR2 of SEQ ID NO:13, and
   (iii) a CDR3 of SEQ ID NO:16;
and,
Antigen binding domain 4, comprising a single variable domain on a heavy chain comprising,
   (i) a CDR1 of SEQ ID NO:19,
   (ii) a CDR2 of SEQ ID NO:21, and
   (iii) a CDR3 of SEQ ID NO:23;
and,
Antigen binding domain 5, comprising a single variable domain on a heavy chain comprising,
   (i) a CDR1 of SEQ ID NO:20,
   (ii) a CDR2 of SEQ ID NO:22, and
   (iii) a CDR3 of SEQ ID NO:24;
b) An antigen binding domain which binds to a T-cell antigen; and
c) A linker joining (a) and (b), suitably a flexible linker.

In embodiments, a bispecific T-cell engager comprising Antibody binding domains 1, 2 and 3 binds (suitably specifically binds) CLL1 and wherein a bispecific T-cell engager comprising Antibody binding domains 4 and 5 binds (suitably specifically binds) CD33.

The antigen binding domain which binds to a T-cell antigen may comprise an antigen binding fragment that specifically binds CD3. Suitably, the antigen binding fragment that specifically binds CD3 comprises:
(a) a heavy chain variable region comprising,
   (i) a CDR1 of SEQ ID NO:67,
   (ii) a CDR2 of SEQ ID NO:68, and
   (iii) a CDR3 of SEQ ID NO:69; and
(b) a light chain variable region comprising,
   (i) a CDR1 of SEQ ID NO:70,
   (ii) a CDR2 of SEQ ID NO:71, and
   (iii) a CDR3 of SEQ ID NO:72.

Also described is an antibody-drug conjugate that binds CLL1 and/or CD33 comprising one or more antigen binding domains selected from the group consisting of:
Antigen binding domain 1, comprising a single variable domain on a heavy chain comprising,
   (i) a CDR1 of SEQ ID NO:8,
   (ii) a CDR2 of SEQ ID NO:11, and
   (iii) a CDR3 of SEQ ID NO:14;
and,
Antigen binding domain 2, comprising a single variable domain on a heavy chain comprising,
   (i) a CDR1 of SEQ ID NO:9,
   (ii) a CDR2 of SEQ ID NO:12, and
   (iii) a CDR3 of SEQ ID NO:15;
and,
Antigen binding domain 3, comprising a single variable domain on a heavy chain comprising,
   (i) a CDR1 of SEQ ID NO:10,
   (ii) a CDR2 of SEQ ID NO:13, and
   (iii) a CDR3 of SEQ ID NO:16;
and,
Antigen binding domain 4, comprising a single variable domain on a heavy chain comprising,
   (i) a CDR1 of SEQ ID NO:19,
   (ii) a CDR2 of SEQ ID NO:21, and
   (iii) a CDR3 of SEQ ID NO:23;
and,
Antigen binding domain 5, comprising a single variable domain on a heavy chain comprising,
   (i) a CDR1 of SEQ ID NO:20,
   (ii) a CDR2 of SEQ ID NO:22, and
   (iii) a CDR3 of SEQ ID NO:24;
b) A therapeutic drug substance;
c) A linker joining (a) and (b).

In embodiments, an antibody-drug conjugate comprising Antibody binding domains 1, 2 and 3 binds (suitably specifically binds) CLL1 and wherein an antibody-drug conjugate comprising Antibody binding domains 4 and 5 binds (suitably specifically binds) CD33.

The therapeutic drug substance may be a cytotoxic drug.

The bispecific T-cell engager, or the antibody-drug conjugate bind CLL1 and/or CD33. Suitably, the bispecific T-cell engager or ADC comprise one of antigen binding domain 1, 2 or 3 and one of antigen binding domain 4 or 5.

Suitably, the bispecific T-cell engager or ADC comprise:
a. Antigen binding domain 1 and antigen binding domain 4; or
b. Antigen binding domain 2 and antigen binding domain 4; or
c. Antigen binding domain 3 and antigen binding domain 5.

In embodiments of the bispecific T-cell engager of the invention, or the antibody-drug conjugate, the single variable domain on a heavy chain of any one of antigen binding domain 1 to 5 is a VHH. Suitably, the VHH is selected from the group consisting of: SEQ ID NOs 5, 6 or 7 and SEQ ID NOs 17 or 18.

In embodiments the bispecific T-cell engager, or the antibody-drug conjugate may be for use as a medicament.

In embodiments, the bispecific T-cell engager, or the antibody-drug conjugate may be for use in killing or inhibiting the growth of cells expressing CLL1 and/or CD33.

In embodiments, the bispecific T-cell engager, or the antibody-drug conjugate may be for use in the treatment of a disease or disorder selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

In embodiments, the invention provides a method of treatment of a disorder or disease, wherein said method comprises administering to a patient in need thereof, the bispecific T-cell engager, or the antibody-drug conjugate.

In embodiments, the disorder or disease is selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

### BRIEF DESCRIPTION OF THE FIGURES

FIG. 1 (A) shows an embodiment of the general structure of the antibody or antigen binding fragment of the present invention (VHH 1) comprising CDRs 1, 2 and 3, within an example single chimeric antigen receptor scaffold comprising a CD8A signal peptide, a CD8A hinge and TM region, CD137 costimulatory domain and CD247 stimulatory domain.

FIG. 1 (B) shows an embodiment of the general structure of the antibodies or antigen binding fragments of the present invention (VHH 1 and VHH 2) each comprising CDRs 1, 2 and 3, within an example tandem chimeric antigen receptor scaffold comprising a CD8A signal peptide, a linker, a CD8A hinge and TM region, CD137 costimulatory domain and CD247 stimulatory domain.

FIG. 1 (C) shows an embodiment of the general structure of the antibodies or antigen binding fragments of the present invention (VHH 1 and VHH 2) each comprising CDRs 1, 2 and 3, within an example parallel chimeric antigen receptor scaffold comprising a CD8A signal peptide, a CD8A hinge and TM region, CD137 costimulatory domain, CD247 stimulatory domain, a T2A self-cleaving peptide, a CD8A signal peptide, a TNFRSF4 hinge and TM domain, CD137 costimulatory domain and CD247 stimulatory domain.

FIG. 2 shows cells transduced with different CLL1/CD33-CAR constructs and expanded for eight days and stained with CLL1-Fc or CD33-Fc. In a second step, cells were stained with PE-conjugated anti-Fc and APC-conjugated anti-CD34 antibodies. Binding is shown as median fluorescence intensity (MFI) of anti-Fc antibody for CD34+ CAR-T cells.

FIG. 3 shows cells transduced with different CLL1/CD33 CAR constructs and expanded for eight days and co-cultured for 24 h with the CD33/CLL1-expressing cancer cell line (HL-60). IFNg production was assessed in the supernatant via IFNg ELISA.

FIG. 4 shows killing capacity of CLL1/CD33-CAR-T cells against A) engineered HeLa cell lines expressing CLL1, CD33 or co-expressing CD33 and CLL1; B) shows killing capacity of CLL1/CD33 CAR-T cells against the HL60 AML cell line, expressing both CLL1 and CD33. CAR-T cells, transduced with anti-CLL1/CD33 CAR constructs or mock transduced T cells, were co-cultured with target cells at a 1:1 ratio for 24 h. Killing was assessed relative to cells cultured in the absence of T cells. Cell death was quantified via ONE-Glo reagent (Promega), with target cells expressing a luciferase reporter gene.

FIG. 5 shows the design optimization of tandem and parallel CARs. A) shows the design of a parallel CAR, based on the parallel CAR comprising antibodies 3 and 5 (Parallel-3/3.1) (SEQ ID NO: 46), where the 2A self-cleaving peptides have been optimized to include a GSG sequence (parallel 3.2) or a dual P2A/T2A sequence to increase protein cleavage and functional CAR expression (parallel 3.3). The schematics for the parallel CARs include the optional additional tCD34 domain and self-cleaving peptide linker. B) shows tandem CARs, where the CAR coding sequence of the tandem CAR comprising antibodies 5 and 3 (tandem-3) (SEQ ID NO: 42) was codon optimized (tandem 3.1) using known methods as described in Fath S, Bauer A P, Liss M, Spriestersbach A, Maertens B, Hahn P, et al. (2011) Multiparameter RNA and Codon Optimization: A Standardized Tool to Assess and Enhance Autologous Mammalian Gene Expression. PLOS ONE 6 (3): e17596. https://doi.org/10.1371/journal.pone.0017596, the contents of which is incorporated herein in its entirety, and in particular the GeneArt® codon optimisation tool (ex ThermoFisher®: www.thermofisher.com/uk/en/home/life-science/cloning/gene-synthesis.html; accessed 8 Feb. 2024), VHH binder order was switched (tandem 3.2) and the G4S linker was reduced in size from 3 repeats to 1 repeat (tandem 3.3).

FIG. 6 shows cells transduced with different tandem CLL1/CD33-CAR constructs and expanded for eight days and stained with CLL1-Fc or CD33-Fc. In a second step, cells were stained with PE-conjugated anti-Fc and APC-conjugated anti-CD34 antibodies. Binding intensity is shown as median fluorescence intensity (MFI) of anti-Fc antibody for CD34+ CAR-T cells.

FIG. 7 shows activation capacity, based on CD25/CD69 upregulation after activation with HL60 cancer cell line, expressing both CLL1 and CD33. Cells were stimulated for 24 h with target cells and subsequently stained with anti-CD34, anti-CD25 and anti-CD69 fluorescence conjugated antibodies. Activation was assessed in CD34+ cells as the percentage of CD25/CD69 double-positive cells.

FIG. 8 shows killing capacity of CLL1/CD33-CAR-T cells against HL60 cancer cell line, expressing both CLL1 and CD33.

FIG. 9 shows cells transduced with different CLL1/CD33 CAR constructs and expanded for eight days and co-cultured for 24 h with the CD33/CLL1-expressing cancer cell line (HL-60). IFNg production was assessed in the supernatant via IFNg ELISA.

### DETAILED DESCRIPTION OF THE INVENTION

The present invention will be described with respect to particular embodiments and with reference to certain drawings but the invention is not limited thereto but only by the claims. Any reference signs in the claims shall not be construed as limiting the scope. The drawings described are only schematic and are non-limiting.

All references cited herein are incorporated by reference in their entirety. Unless otherwise defined, all technical and scientific terms used herein have the same meaning as commonly understood by one of ordinary skill in the art to which this invention belongs.

Prior to further setting forth the invention, a number of definitions are provided that will assist in the understanding of the invention.

The articles ‘a’, ‘an’ and ‘the’ are used to refer to one or to more than one (i.e. to at least one) of the grammatical object of the article.

As used herein, the term ‘comprising’ means any of the recited elements are necessarily included and other elements may optionally be included as well. ‘Consisting essentially of’ means any recited elements are necessarily included, elements which would materially affect the basic and novel characteristics of the listed elements are excluded, and other elements may optionally be included. ‘Consisting of’ means that all elements other than those listed are excluded. Embodiments defined by each of these terms are within the scope of this invention.

Unless specifically defined herein, all terms used herein have the same meaning as they would to one skilled in the art of the present invention. Practitioners are particularly directed to Green and Sambrook, Molecular Cloning: A Laboratory Manual, 4th ed., Cold Spring Harbor Laboratory Press, New York (2012); and Ausubel et al., Current Protocols in Molecular Biology (up to Supplement 114), John Wiley & Sons, New York (2016), for definitions and terms of the art. The definitions provided herein should not be construed to have a scope less than understood by a person of ordinary skill in the art.

The term ‘antibody’ refers to all isotypes of immunoglobulins (IgG, IgA, IgE, IgM, IgD, and IgY) including various monomeric, polymeric and chimeric forms, unless otherwise specified. Specifically encompassed by the term ‘antibody’ are polyclonal antibodies, monoclonal antibodies (mAbs), single domain antibodies, human (FHVH) or heavy-chain antibodies found in camelids (VHH) and antibody-like polypeptides, such as chimeric antibodies and humanized antibodies. The term ‘monoclonal antibody’ refers to an antibody produced by a single clone of cells or cell line and consisting of identical antibodies. As used herein, the term ‘antibody’ or ‘antigen-binding fragments’ may encompass any protein structure that exhibits binding affinity for a particular antigen, or more than one particular antigen.

Antigen-binding fragments include those provided by any known technique, such as enzymatic cleavage, peptide synthesis, and recombinant techniques. Some antigen-binding fragments are composed of portions of intact antibodies that retain antigen-binding specificity of the parent antibody molecule. For example, antigen-binding fragments may comprise at least one variable region (either a heavy chain or light chain variable region) or one or more CDRs of an antibody known to bind a particular antigen. Examples of suitable antigen-binding fragments include, without limitation diabodies, nanobodies and single-chain molecules as well as Fab, F(ab′)2, Fc, Fabc, and Fv molecules, single chain (sc) antibodies, individual antibody light chains, individual antibody heavy chains, chimeric fusions between antibody chains or CDRs and other proteins, protein scaffolds, heavy chain monomers or dimers, light chain monomers or dimers, dimers consisting of one heavy and one light chain, a monovalent fragment consisting of the VL, VH, CL and CH1 domains, or a monovalent antibody as described in WO2007059782, bivalent fragments comprising two Fab fragments linked by a disulfide bridge at the hinge region, a Fd fragment consisting essentially of the VH and CH1 domains; a Fv fragment consisting essentially of the VL and VH domains of a single arm of an antibody, a dAb fragment (Ward et al., Nature 341, 544-546 (1989)), which consists essentially of a VH domain and also called domain antibodies (Holt et al; Trends Biotechnol. 2003 November; 21 (11): 484-90); camelid or nanobodies (Revets et al; Expert Opin Biol Ther. 2005 January; 5 (1): 111-24); an isolated complementarity determining region (CDR), and the like. All antibody isotypes may be used to produce antigen-binding fragments. Additionally, antigen-binding fragments may include non-antibody proteinaceous frameworks that may successfully incorporate polypeptide segments in an orientation that confers affinity for a given antigen of interest, such as protein scaffolds. Antigen-binding fragments may be recombinantly produced or produced by enzymatic or chemical cleavage of intact antibodies. The phrase ‘an antibody or antigen-binding fragment’ may be used to denote that a given antigen binding fragment incorporates one or more amino acid segments of the antibody referred to in the phrase.

As used herein the term ‘antigen binding domain’ refers to a peptide sequence that is intended or able to bind a target of interest. In examples, the antigen binding domain is an antigen-binding fragment as defined above. All types of antigen binding domains are encompassed by the present invention. Examples of some antigen binding domains are scFvs, VHH single domain antibodies or nanobodies, and antigen binding fragments.

The term “VHH” or “V_{H}H” refers to a single-domain antibody (sdAb), also known as a nanobody, and is an antibody fragment consisting of a single monomeric variable antibody domain located on a heavy chain.

An ‘scFv’ or ‘single chain variable fragment’ as used herein, refers to a type of antigen binding domain. Typically, an scFv is a fusion of the variable regions of the heavy (VH) and light chains (VL) of an antibody for a given target connected by a short linker.

Antigen binding domains may comprise ‘CDRs’ or ‘complementarity determining regions’ which are predominantly responsible for target binding. On a typical antibody, multiple CDRs exist and may be selected or varied independently to achieve multiple points of diversity. A ‘recognition sequence’ refers to the nucleic acid sequence encoding one or more antigen binding domains.

‘Specific binding’ or ‘immunospecific binding’ or derivatives thereof when used in the context of antibodies, or antibody fragments, represents binding via domains encoded by immunoglobulin genes or fragments of immunoglobulin genes to one or more epitopes of a protein of interest, without preferentially binding other molecules in a sample containing a mixed population of molecules. Typically, an antibody binds to a cognate antigen with a Kd of less than about 1×10⁻⁸ M, as measured by a surface plasmon resonance assay or a cell binding assay. Phrases such as ‘[antigen]-specific’ antibody (e.g. CLL1-specific antibody) are meant to convey that the recited antibody specifically binds the recited antigen.

As used herein, the term ‘isolated’ means a biological component (such as a nucleic acid, peptide or protein) has been substantially separated, produced apart from, or purified away from other biological components of the organism in which the component naturally occurs, i.e., other chromosomal and extrachromosomal DNA and RNA, and proteins. Nucleic acids, peptides and proteins that have been isolated thus include nucleic acids and proteins purified by standard purification methods. Isolated nucleic acids, peptides and proteins can be part of a composition and still be isolated if such composition is not part of the native environment of the nucleic acid, peptide, or protein. The term also embraces nucleic acids, peptides and proteins prepared by recombinant expression in a host cell as well as chemically synthesized nucleic acids. An ‘isolated’ antibody or antigen-binding fragment, as used herein, is intended to refer to an antibody or antigen-binding fragment which is substantially free of other antibodies or antigen-binding fragments having different antigenic specificities (for instance, an isolated antibody that specifically binds to CLL1 is substantially free of antibodies that specifically bind antigens other than CLL1). An isolated antibody that specifically binds to an epitope, isoform or variant of CLL1 may, however, have cross-reactivity to other related antigens, for instance from other species (such as CLL1 species homologs).

As used herein, the term ‘chimeric antigen receptor’ or ‘CAR’ refers to transmembrane receptor that has been engineered to target or bind to a non-native substrate or antigen. In this way the intracellular signalling of the receptor may triggered by binding of a non-native substrate or antigen. Typically, the term CAR refers to a chimeric receptor (i.e. a receptor composed of two or more parts from different sources) that has at least a binding moiety or recognition sequence with a specificity for a target such as an antigen or protein and an intracellular signaling (or cytosolic) domain that can invoke a signal in the cell in which the CAR is expressed (e.g. a CD247 (CD3 zeta) chain). In embodiments, a ‘chimeric antigen receptor’ or ‘CAR’ is formed of at least three domains: an extracellular antigen binding domain (as defined elsewhere herein), a transmembrane domain and an intracellular domain. A hinge domain between the antigen binding domain and the transmembrane domain is often used to improve recognition of the target. A costimulatory domain may optionally be present in the intracellular domain to modulate the response. To be functional, the domains of the CAR must be ordered correctly. CARs are often used on T-cells (to produce ‘CAR T-cells’) to effect recognition and elicit an appropriate intracellular response which both binds the T-cell to a target cell and triggers the innate immune response of the T-cell, typically causing lysis of the target cell. Such cells have found use in therapy. The term a ‘tandem CAR’ is used herein to refer to a CAR with one or more, suitably two, antigen binding fragments in a single antigen binding domain, optionally joined by a linker, suitable a single or multi-repeat GGGGS (4GS) linker. The term a ‘parallel CAR’ is used herein to refer to a CAR sequence (or CARs derived from that sequence) that comprises two or more CAR sequences joined by a linker, suitably a self-cleaving peptide linker, such that following translation, two or more CARs are expressed from the separate CAR sequences.

A ‘transmembrane domain’ or ‘TM domain’ as used herein is any membrane-spanning protein domain. Suitably, the TM domain in a CAR is derived from a known transmembrane protein sequence. However, it can also be artificially designed. A ‘transmembrane sequence’ refers to the nucleic acid sequence encoding a transmembrane domain.

As used herein the term ‘hinge domain’ refers to a peptide sequence that connects the antigen binding domain and transmembrane region of a CAR. The hinge domain is located between the antigen binding fragment and the T cell plasma membrane (Moritz D, et al. Gene Ther. 1995; 2 (8): 539-46)

The term ‘signaling domain’ or ‘intracellular domain’ or ‘intracellular signaling domain’ as used herein refers to a moiety that can transmit a signal in a cell, for example an immune cell. The signaling domain typically comprises a domain derived from a receptor that signals by itself in immune cells, such as the T Cell Receptor (TCR) complex or the Fc receptor or DAP10/DAP12 receptors. Additionally, it may contain a costimulatory domain (i.e. a domain derived from a receptor that is required in addition to the TCR to obtain full activation of T cells). The costimulatory domain can be from an activating costimulatory receptor or from an inhibitory costimulatory receptor. An ‘intracellular domain sequence’ refers to the nucleic acid sequence encoding an intracellular signaling domain.

The term ‘self-cleaving peptide’ or ‘self-cleaving peptide sequence’ as used herein means a peptide or peptide sequence that encodes a protein capable of undergoing a cleavage reaction or breaks translation under certain conditions, resulting in the separation of specific functional groups or domains in a larger peptide chain (Liu, Z., et al. Systematic comparison of 2A peptides for cloning multi-genes in a polycistronic vector. Sci Rep 7, 2193 (2017); Wang, Y., Wang, F., Wang, R. et al. 2A self-cleaving peptide-based multi-gene expression system in the silkworm Bombyx mori. Sci Rep 5, 16273 (2015)). The term can mean ‘2A self-cleaving peptides’ or ‘2A peptides’ that are a class of short (18-22 amino acid) peptides that can induce ribosomal skipping during translation of a protein. 2A peptides share a core sequence motif of DxExNPGP which causes the ribosome to fail to make a peptide bond to the next amino acid resulting in cleavage. Four members of 2A peptides family are frequently used: P2A (ATNFSLLKQAGDVEENPGP), E2A (QCTNYALLKLAGDVESNPGP), F2A (VKQTLNFDLLKLAGDVESNPGP), and T2A (EGRGSLLTCGDVEENPGP). Elaboration of the conserved sequence, for example at the start of the sequence, is possible and adding an optional “GSG” at the N-terminal of a 2A peptide can help with cleavage efficiency.

As used herein, the terms ‘bi-specific’, ‘tri-specific’ or ‘multi-specific’ refer to an antibody molecule (i.e. an antibody or antigen binding fragment conjugated to a synthetic molecule) that comprises one or more further antigen binding domains such that the antibody molecule can have specificity for more than one antigen.

As is known in the art, as used herein, the term ‘bispecific T-cell engager’ or ‘BiTE’ is used synonymously to refer to a single polypeptide chain molecule having two antigen binding domains, one of which binds to a T-cell antigen (e.g., CD3) and the second of which binds to an antigen present on the surface of a target cell (e.g. CLL1), these two antigen binding domains separated by a linker, specifically a flexible linker, more specifically a flexible protein linker (WO 05/061547; Baeuerle, et al. (2008) Drugs of the Future 33:137-147; Bargou, et al. (2008) Science 321:974-977). BiTE antibodies have been constructed to various target antigens including CD19, EpCAM, Her2/neu, EGFR, CD66e (or CEA, CEACAM5), CD33, EphA2, and MCSP (or HMW-MAA) (Baeuerle, et al. (2009) Curr. Opin. Mol. Ther. 11:22-30). Key hallmarks of BiTE antibodies that, in their combination, distinguish them from other bispecific antibody constructs, include a high potency of redirected lysis with EC50 values ranging from 0.1 to 50 μmol/L (2-1,000 μg/mL) (Baeuerle, et al. (2009) supra); strict target cell-dependent activation of T cells (Brischwein, et al. (2007) J. Immunother. 30:798-807); and support of serial lysis by activated T cells, i.e., activity at low E: T ratios. BiTE antibodies are typically produced as recombinant, glycosylated proteins secreted by higher eukaryotic cell lines. The terms ‘bi-specific T-Cell engager’ and/or ‘BiTE’ as herein defined encompass, but are not limited to, the specific bi-specific T-Cell engager structure encompassed by the trademark ‘BITER’ in the name of Amgen Research (Munich) GmbH.

The term ‘DART™ ’ (Dual Affinity Re-Targeting reagent) diabody refers to an immunoglobulin molecule that comprises at least two polypeptide chains that associate (for example, through a covalent interaction) to form at least two antigen binding domains, which may recognize the same or different antigens. Each of the polypeptide chains of a DART™ diabody comprise an immunoglobulin light chain variable region and an immunoglobulin heavy chain variable region, but these regions do not interact to form an antigen binding domain. Rather, the immunoglobulin heavy chain variable region of one (e.g., the first) of the DART™ diabody polypeptide chains interacts with the immunoglobulin light chain variable region of a different (e.g. the second) DART™ polypeptide chain to form an antigen binding domain. Similarly, the immunoglobulin light chain variable region of one (e.g., the first) of the DART™ diabody polypeptide chains interacts with the immunoglobulin heavy chain variable region of a different (e.g., the second) DART™ diabody polypeptide chain to form an antigen binding domain. DART™ diabodies may be monospecific, bispecific, trispecific, etc., thus being able to simultaneously bind one, two, three or more different antigens (which may be of the same or of different antigens). DART™ diabodies may additionally be monovalent, bivalent, trivalent, tetravalent, pentavalent, hexavelent, etc., thus being able to simultaneously bind one, two, three, four, five, six or more molecules. These two attributes of DART™ diabodies i.e. degree of specificity and valency, may be combined, for example to produce bispecific antibodies i.e. capable of binding two antigens, that are tetravalent i.e., capable of binding four sets of epitopes, etc. DART™ diabody molecules are disclosed in PCT applications published as WO 2006/113665, WO 2008/157379, and WO 2010/080538.

As used herein, the term ‘antibody-drug conjugate’ or ‘ADC’ means a biopharmaceutical drug that comprises an antibody or antigen binding fragment, and a drug substance, typically a cytotoxic drug substance, connected via a linker, suitably a covalent, chemical linker.

As used herein, the term ‘high-throughput screening’ refers to any assay or screening methodology that allows for a higher rate of screening than would be achieved by traditional or previous state of the art techniques. Typically, high-throughput screening enables automation to prepare, screen and/or evaluate libraries of test samples in parallel, reproducibly and rapidly. High-throughput screening can also make use of combinatorial or pooled or mixed sample screening strategies, with associated deconvolution of hits. In the context of CAR high throughput screening, the number of samples for test may be of any size larger than that typically used in prior art non-high-throughput methods. For example, the number of samples may be more than 10. Suitably the number of samples may be more than 11, 12, 13, 14, 15, 16, 17, 18, 19, 20, 30, 40, 50, 100, 200 or more.

As used herein, the term ‘recognition sequence library’ refers to a set of one or more antigen binding domain sequences or recognition sequences that may be used for cloning into a CAR construct (wherein a CAR construct comprises all components required for a functioning CAR including one or more antigen binding domains, a hinge domain, a transmembrane domain and an intracellular domain) to prepare a CAR library. An ‘scFv library’ is a recognition sequence library formed of scFv recognition sequences from antibodies. A ‘VHH library’ is a recognition sequence library formed of VHH recognition sequences from antibodies. In embodiments, the number of recognition sequences present in the recognition sequence library may be more than 1 or more than 2. Suitably the number of hinge region sequences present in the hinge region sequence library may be more than 3, 4, 5, 6, 7, 8, 9, 10, 11, 12, 13, 14, 15, 16, 17, 18, 19, 20, 30, 40, 50, 100, 200 or more.

As used herein, the term ‘CAR scaffold’ refers to the part of the CAR sequence that comprises the components, parts, modules, domains of the CAR excluding the antigen binding domain, that form the sequence of a CAR in a CAR library. The CAR scaffold is formed of the sequences of the individual components, or groups thereof, and combined with the recognition sequence to form a sequence encoding a CAR in any suitable manner, for example, sequentially, convergently, with the recognition sequence being incorporated at any suitable point, i.e. the recognition sequence may be joined to a single unitary sequence of the CAR scaffold, or may be joined to a component part of the CAR scaffold initially and the full CAR sequence of the recognition sequence and scaffold completed subsequently. The component parts of the CAR scaffold may derive from single sequences leading to one or a small number of CAR scaffolds for addition to a recognition sequence, or the component parts of the CAR scaffold may derive from libraries of one or more of the individual components leading to a CAR scaffold library comprising a plurality of sequences. A CAR scaffold library may be formed by combination, suitably in a combinatorial, or directed, manner of the individual components or component libraries.

As used herein, the term ‘CAR library’ refers to a set of sequences encoding a functional CAR structure, including at least one recognition domain, such as an scFv or VHH, a hinge domain, a transmembrane domain and intracellular domain(s). In embodiments, the number of CARs present in the CAR library may be more than 10. Suitably the number of CARs present in the CAR library may be more than 11, 12, 13, 14, 15, 16, 17, 18, 19, 20, 30, 40, 50, 100, 200 or more. The term ‘CAR library’ may also be used interchangeably to refer to the plasmid or vector, or otherwise modified sequence of the sequence(s) encoding for a functional CAR structure.

As used herein, the term ‘CAR-cell library’ refers to a collection or set of cells expressing CARs on their surface. Each cell in the CAR-cell library may express a single CAR (i.e. express only one CAR encoded by a single sequence), or a single cell in the CAR-cell library may express two or more CARs, each encoded by the same or different sequences. Suitably, each cell in the CAR-cell library expresses a single CAR or all CARs present in the library. In embodiments, the number of CARs present in the CAR-cell library may be more than 10. Suitably the number of CARs present in the CAR-cell library may be more than 11, 12, 13, 14, 15, 16, 17, 18, 19, 20, 30, 40, 50, 100, 200 or more.

As used herein, the term ‘next generation sequencing’ of ‘NGS’ refers to a catch-all term used to describe a number of different modern sequencing technologies. These technologies allow for sequencing of DNA and RNA much more quickly and cheaply than the previously used Sanger sequencing. Examples are Solex™ sequencing by Illumina™, Roche 454™ sequencing, Ion Torrent™ sequencing and nanopore based sequencing methods (e.g. Oxford Nanopore Technologies™ Grid ION™).

The phrase ‘nucleic acid molecule’ synonymously referred to as ‘nucleotides’ or ‘nucleic acids’ or ‘polynucleotide’ refers to any polyribonucleotide or polydeoxyribonucleotide, which may be unmodified RNA or DNA or modified RNA or DNA. Nucleic acid molecules include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, ‘polynucleotide’ refers to triple-stranded regions comprising RNA or DNA or both RNA and DNA. The term polynucleotide also includes DNAs or RNAs containing one or more modified bases and DNAs or RNAs with backbones modified for stability or for other reasons. ‘Modified’ bases include, for example, tritylated bases and unusual bases such as inosine. A variety of modifications may be made to DNA and RNA; thus, ‘polynucleotide’ embraces chemically, enzymatically or metabolically modified forms of polynucleotides as typically found in nature, as well as the chemical forms of DNA and RNA characteristic of viruses and cells. ‘Polynucleotide’ also embraces relatively short nucleic acid chains, often referred to as oligonucleotides.

There are various means by which a nucleic acid sequence may be inserted into a genome, including but not limited to plasmid or vector transfection, transposition and genome editing. All are contemplated for use in the present invention.

As used herein a ‘vector’ is a replicon, such as plasmid, phage, cosmid, or virus in which another nucleic acid segment may be operably inserted so as to bring about the replication or expression of the segment. A ‘transposon’ or ‘transposable elements’ are DNA sequences that can change their position within a genome. ‘Genome editing’ refers to the ability to edit the genome to insert the required sequence, for example using CRISPR-Cas9 genome editing technology.

A ‘clone’ is a population of cells derived from a single cell or common ancestor by mitosis.

A ‘cell line’ is a clone of a primary cell that is capable of stable growth in vitro for many generations. In some examples provided herein, cells are transformed by transfecting the cells with DNA.

The terms ‘express’ and ‘produce’ are used synonymously herein and refer to the biosynthesis of a gene product. These terms encompass the transcription of a gene into RNA. These terms also encompass translation of RNA into one or more polypeptides, and further encompass all naturally occurring post-transcriptional and post-translational modifications.

The term ‘subject’ refers to human and non-human animals, including all vertebrates, e.g., mammals and non-mammals, such as non-human primates, mice, rabbits, sheep, goats, dogs, cats, horses, cows, chickens, amphibians, and reptiles. In most particular embodiments of the described methods, the subject is a human.

The terms ‘treating’ or ‘treatment’ refer to any success or indicia of success in the attenuation or amelioration of an injury, pathology or condition, including any objective or subjective parameter such as abatement, remission, diminishing of symptoms or making the condition more tolerable to the patient, slowing in the rate of degeneration or decline, making the final point of degeneration less debilitating, improving a subject's physical or mental well-being, or prolonging the length of survival. The treatment may be assessed by objective or subjective parameters; including the results of a physical examination, neurological examination, or psychiatric evaluations.

### Sequence Homology

When referring to CLL1 and/or CD33, it will be appreciated that reference thereto encompasses fragments thereof, as well as related polypeptides, which include, but are not limited to, allelic variants, splice variants, derivative variants, substitution variants, deletion variants, and/or insertion variants including the addition of an N-terminal methionine, fusion polypeptides, and interspecies homologs. In certain embodiments, a CLL1 and/or CD33 polypeptide includes terminal residues, such as, but not limited to, leader sequence residues, targeting residues, amino terminal methionine residues, lysine residues, tag residues and/or fusion protein residues.

The amino acid sequence of an example isoform of CLL1 to which the antibodies of the present invention bind is provided as SEQ ID NO: 1.

The DNA sequence of CLL1 (SEQ ID NO: 1) is provided SEQ ID NO: 2. Splice variants and other isoforms of CLL1 are known and are encompassed by the present disclosure.

The amino acid sequence of an example isoform of CD33 to which the antibodies of the present invention bind is provided as SEQ ID NO: 3.

The DNA sequence of CD33 (SEQ ID NO: 3) is provided SEQ ID NO: 4. Splice variants and other isoforms of CD33 are known and are encompassed by the present disclosure.

For all aspects and embodiments of the present invention, the amino acid sequence of CLL1 encompasses a polypeptide that has at least 50%, 60%, 70%, 80%, 90%, 95% or 99% complete sequence identity to CLL1 (for example SEQ ID NO: 1). Likewise, the CLL1 polynucleotide sequence can comprise a polynucleotide that has at least 50%, 60%, 70%, 80%, 90%, 95% or 99% complete sequence identity to CLL1 (for example, SEQ ID NO: 2). Sequence identity can also be to a fragment or portion of the full-length polynucleotide or polypeptide. Hence, a sequence may have only 50% overall sequence identity with a sequence of the invention but in a particular region, domain or subunit could share 80%, 90%, or as much as 99% sequence identity with sequence of the invention.

For all aspects and embodiments of the present invention, the amino acid sequence of CD33 encompasses a polypeptide that has at least 50%, 60%, 70%, 80%, 90%, 95% or 99% complete sequence identity to CD33 (for example SEQ ID NO: 3). Likewise, the CD33 polynucleotide sequence can comprise a polynucleotide that has at least 50%, 60%, 70%, 80%, 90%, 95% or 99% complete sequence identity to CD33 (for example, SEQ ID NO: 4). Sequence identity can also be to a fragment or portion of the full-length polynucleotide or polypeptide. Hence, a sequence may have only 50% overall sequence identity with a sequence of the invention but in a particular region, domain or subunit could share 80%, 90%, or as much as 99% sequence identity with sequence of the invention.

According to the present invention, homology to a nucleic acid sequence is not limited simply to sequence identity. Many nucleic acid sequences can demonstrate biologically significant homology to each other despite having apparently low sequence identity. In the present invention homologous nucleic acid sequences are considered to be those that will hybridise to each other under conditions of low stringency (Sambrook J. et al, Molecular Cloning: A Laboratory Manual, Cold Spring Harbor Press, Cold Spring Harbor, NY).

For all aspects and embodiments of the present invention, the nucleic acid sequence of the antigen binding fragments or the antigen binding molecules of the present invention (e.g. antibodies, antigen binding fragments, CARs, BiTEs. ADCs) encompasses a nucleic acid that has at least 80%, 90%, 95% or 99% complete sequence identity to the sequences provided (for example SEQ ID NO: 3, and SEQ ID NOS. 73-85, 97-99, 107-111). Sequence identity can also be to a fragment or portion of the full-length polynucleotide. Hence, a sequence may have only 50% overall sequence identity with a sequence of the invention but in a particular region, domain or subunit could share 80%, 90%, or as much as 99% sequence identity with sequence of the invention.

The present application shows a systematic high-throughput screen output of a CAR-T cell library. This allowed identification of multiple novel antibody or antigen binding fragments (e.g. VHHs) and their associated heavy chain CDRs, that bind to CLL1 and/or CD33, and outperform other known antibody or antigen binding fragments in several experimental models.

Accordingly, in at least one aspect, the invention provides an antibody or antigen binding fragments having specificity for CLL1 (Antibody Nos. 1 to 3), wherein the antibody or antigen binding fragments have an antigen binding domain comprising the amino acid sequences (SEQ ID NOS: 5 to 16) as shown in Table 1 below:

TABLE 1
Exemplified CLL1 antibodies or antigen binding fragments of the present invention

| Antibody | | VHH | | |
|---|---|---|---|---|
| No. | VHH | CDR1 | CDR2 | CDR3 |
| 1 | SEQ ID NO. 5 | SEQ ID NO.8: | SEQ ID NO.11: | SEQ ID NO.14: |
| | | GFTFSTYD | ISGSGGTT | VRGDDH |
| 2 | SEQ ID NO. 6 | SEQ ID NO.9: | SEQ ID NO.12: | SEQ ID NO.15: |
| | | GFTFSNYD | ITGSGGST | AAGVDGDY |
| 3 | SEQ ID NO. 7 | SEQ ID NO.10: | SEQ ID NO.13: | SEQ ID NO.16: |
| | | GFTFSAYD | ISGSGGST | AYDGDSTGSLD |
| | | | | EMAH |

In a further aspect, the invention provides an antibody or antigen binding fragments having specificity for CD33 (Antibody Nos. 4 to 5), wherein the antibody or antigen binding fragments have an antigen binding domain comprising the amino acid sequences (SEQ ID NOS: 17 to 24) as shown in Table 2 below:

TABLE 2
Exemplified CD33 antibodies or antigen binding fragments of the present invention

| Antibody | | VHH | | |
|---|---|---|---|---|
| No. | VHH | CDR1 | CDR2 | CDR3 |
| 4 | SEQ ID NO. 17 | SEQ ID NO. 19: | SEQ ID NO. 21: | SEQ ID NO. 23: |
| | | GFTFSTYA | IEGSGGST | AVETGWADFDY |
| 5 | SEQ ID NO. 18 | SEQ ID NO. 20: | SEQ ID NO. 22: | SEQ ID NO. 24: |
| | | GFTFSYYA | IEGSGGRT | VKETGWADFDY |

In a further aspect, the invention provides a CAR having specificity for CLL1 and/or CD33 (single VHH CARs), wherein the CARs comprise the amino acid sequences (SEQ ID NOS: 25 to 31) as shown in Table 3 below (numbers in the table relate to SEQ ID NOS):

TABLE 3
Exemplified CLL1/CD33 single VHH CARs of the present invention

| | | | Domains | | | | |
|---|---|---|---|---|---|---|---|
| | | | | | | Co- | Intra- |
| | Protein | DNA | Signal | | Hinge/TM | stimulatory | cellular |
| CAR | Seq | Seq | Peptide | VHH | domain | domain | domain |
| Single-1 | 25 | 73 | 39 | 5 | 34 | 33 | 32 |
| Single-2 | 26 | 74 | 39 | 6 | 34 | 33 | 32 |
| Single-3 | 27 | 75 | 39 | 7 | 34 | 33 | 32 |
| Single-4a | 28 | 76 | 39 | 17 | 34 | 33 | 32 |
| Single-4b | 30 | 78 | 39 | 17 | 35 | 33 | 32 |
| Single-5a | 29 | 77 | 39 | 18 | 34 | 33 | 32 |
| Single-5b | 31 | 79 | 39 | 18 | 35 | 33 | 32 |

In a further aspect, the invention provides a tandem CAR comprising two VHH joined by a linker having specificity for CLL1 and CD33 (tandem CARs), wherein the CARs comprise the amino acid sequences (SEQ ID NOS: 40 to 42 and 93 to 94) as shown in Table 4 below:

TABLE 4
Exemplified CLL1/CD33 tandem VHH CARs of the present invention

| | | | Domains | | | | | | |
|---|---|---|---|---|---|---|---|---|---|
| | | | | | | | | | Intra- |
| | Protein | DNA | Signal | | | | Hinge/TM | Co-stim | cellular |
| CAR | Seq | Seq | Peptide | VHH1 | Linker | VHH2 | domain | domain | domain |
| Tandem-1 | 40 | 80 | 39 | 5 | 36 | 17 | 34 | 33 | 32 |
| Tandem-2 | 41 | 81 | 39 | 6 | 36 | 17 | 34 | 33 | 32 |
| Tandem-3 | 42 | 82 | 39 | 18 | 36 | 7 | 34 | 33 | 32 |
| Tandem-3.1 | 42 | 97 | 39 | 18 | 36 | 7 | 34 | 33 | 32 |
| Tandem-3.2 | 93 | 98 | 39 | 18 | 36 | 7 | 34 | 33 | 32 |
| Tandem-3.3 | 94 | 99 | 39 | 18 | 36 | 7 | 34 | 33 | 32 |

The CARs labelled Tandem-3.1, Tandem-3.2 and Tandem-3.3 are based on the CAR labelled Tandem-3, with further codon and sub-unit optimisation to improve expression and functionality. The protein sequence of the antigen binding region is unchanged.

The CARs labelled Tandem-3 and Tandem-3.1 have an identical CAR protein sequence but the DNA sequence of Tandem-3.1 has been optimised and therefore differs from the DNA sequence of Tandem-3.

In a further aspect, the invention provides a parallel CAR comprising two full CAR sequences joined by a self-cleaving linker each CAR having specificity for CLL1 or CD33 (parallel CARs), wherein the CARs comprise the amino acid sequences (SEQ ID NOS: 44 to 46 and 95 to 96) as shown in Table 5 below:

TABLE 5
Exemplified CLL1/CD33 tandem VHH CARs of the present invention.

| | | | Domains | | | | | |
|---|---|---|---|---|---|---|---|---|
| | | | | | | | Intra- | Self- |
| | Protein | DNA | Signal | | Hinge/TM | Co-stim | cellular | cleaving |
| CAR | Seq | Seq | Peptide | VHH | domain | domain | domain | linker** |
| Parallel-1 | 44 | 83 | 1:39* | 1:5 | 1:34 | 1:33 | 1:32 | 43 |
| | | | 2:39 | 2:17 | 2:35 | 2:33 | 2:32 | |
| Parallel-2 | 45 | 84 | 1:39 | 1:6 | 1:34 | 1:33 | 1:32 | 43 |
| | | | 2:39 | 2:17 | 2:35 | 2:33 | 2:32 | |
| Parallel-3 | 46 | 85 | 1:39 | 1:7 | 1:34 | 1:33 | 1:32 | 43 |
| | | | 2:39 | 2:18 | 2:35 | 2:33 | 2:32 | |
| Parallel-3.2 | 95 | 100 | 1:39 | 1:7 | 1:34 | 1:33 | 1:32 | 88 |
| | | | 2:39 | 2:18 | 2:35 | 2:33 | 2:32 | |
| Parallel-3.3 | 96 | 101 | 1:39 | 1:7 | 1:34 | 1:33 | 1:32 | 89 |
| | | | 2:39 | 2:18 | 2:35 | 2:33 | 2:32 | |

*“1:” relates to CAR 1 or the CAR closer to the N-terminus of the full protein sequence; “2:” relates to CAR 2 or the CAR nearer the C-terminus of the protein sequence;
**“Self-cleaving linker relates to the peptide sequence linking CAR 1 to CAR 2.

Each of the CARs above may comprise one or more additional domain to facilitate or validate expression, or other biological activity. Such additional domains may suitably be linked to the main CAR sequence via a self-cleaving peptide. Suitably, the additional domain is positioned at the C-terminus end, separated from the main CAR sequence by the self-cleaving peptide. An example of an additional domain of this type, as exemplified in the examples, is tCD34.

While the additional domain is optional and is not an essential feature of the present invention, when present, along with the self-cleaving linker, it can have an effect on expression of the CAR and other factors affecting performance of the CAR. All of the CARs with a single VHH (Single-1 to Single-5b) and the tandem CARs (Tandem-1 to Tandem-3.3) have the same N-terminus domain, linked by a self-cleaving peptide, with the sequences:

Self-cleaving (2A) peptide:
(SEQ ID NO. 90)
ARAKRSGSGEGRGSLITCGDVEENPGP
tCD34:
(SEQ ID NO. 92)
MPRGWTALCLLSLLPSGFMSLDNNGTATPELPTQGTFSNVSTNVSYQETTTPSTLGSTSLHP
VSQHGNEATTNITETTVKFTSTSVITSVYGNTNSSVQSQTSVISTVFTTPANVSTPETTLKPSLSPGNV
SDLSTTSTSLATSPTKPYTSSSPILSDIKAEIKCSGIREVKLTQGICLEQNKTSSCAEFKKDRGEGLARV
LCGEEQADADAGAQVCSLLLAQSEVRPQCLLLVLANRTEISSKLQLMKKHQSDLKKLGILDFTEQDVA
SHQSYSQKTLIALVTSGALLAVLGITGYFLMNRRSWSPTGERLELEP

TABLE 6
The parallel CARs of the present invention each have the
following C-terminus additional domains (Table 6):
Exemplified CLL1/CD33 parallel VHH CARs of the present
invention with the attached tCD34 C-terminus domain.

| | Full Protein | Full DNA | | |
|---|---|---|---|---|
| | Sequence | Sequence | Additional Domain | |
| | (inc. additional | (inc. additional | Self- | |
| | C-terminus | C-terminus | cleaving | |
| CAR | domain) | domain) | peptide-2* | tCD34 |
| Parallel-1 + tCD34 | 102 | 107 | 90 | 92 |
| Parallel-2 + tCD34 | 103 | 108 | 90 | 92 |
| Parallel-3 + tCD34 | 104 | 109 | 90 | 92 |
| Parallel-3.2 + tCD34 | 105 | 110 | 91 | 92 |
| Parallel-3.3 + tCD34 | 106 | 111 | 89 | 92 |

*Self-cleaving peptide-2 denotes that this is the self-cleaving peptide that links the terminal intracellular domain of the CAR to the tCD34 domain, and not the self-cleaving peptide indicated in Table 5 that links the CAR 1 and CAR 2 sequences in the parallel CAR motif.

The protein sequence of the antibodies, or antigen binding fragments, of the present invention were identified from a functional high-throughput screen of CAR-T cells, wherein the CAR-T cells express a unique chimeric antigen receptor (CARs), each CAR comprising an antibody or antigen binding fragment and/or VHH derived from a diverse VHH library. The antibodies or antigen binding fragments as identified in Tables 1 and 2 and the associated CARs in Tables 3 to 6 are those exhibiting surprisingly good functional response (i.e. were positive hits) in various assays against cells expressing CLL1 and/or CD33 and are therefore deemed to be superior CAR-T cells over that known in the art and provide novel and effective antibodies or antigen binding fragments of CLL1 and/or CD33.

The high-throughput screening method employed to identify antibodies or antigen binding fragments, and/or bi-specific, tri-specific or multi-specific antigen binding molecules and/or antibody-drug conjugates and/or CAR-T cells derived therefrom is described in the Applicant's co-pending International application PCT/GB2022/050158 (published as WO2022/157500), the content of which is incorporated by reference.

In embodiments, the antibody or antigen binding fragment can be an isolated monospecific or bispecific antibody having specificity for CLL1 (suitably human CLL1) and/or CD33 (suitably human CD33) and can be a full-length antibody or an antibody fragment. The antibody can be polyclonal, monoclonal, recombinant, chimeric, or humanised. Furthermore, the antibody can be of any isotype including without limitation IgA, IgD, IgE, IgG, or IgM. Thus, for example, the antibody can be any IgA such as IgA1 or IgA2, or any IgG such as IgG1, IgG2, IgG3, IgG4, or synthetic IgG. The antibody can also be any antibody fragment having specificity for CLL1 and/or CD33, such as F (ab) 2, Fv, scFv, F(ab′) 2, F (ab), VL, VH, VHH, dsFv, Fv, scFv-Fc, (scFv) 2, a diabody, and a bivalent antibody. The antibody can be any modified or synthetic antibody, including, but not limited to, non-depleting IgG antibodies, T-bodies, or other Fe or Fab variants of antibodies.

In some embodiments, the invention provides an antibody or antigen binding fragment with avidity for CLL1 and/or CD33 of about 10 UM or less, 5 UM or less, 2 UM or less, 1 μM or less, 500 nM or less, 400 nM or less, 300 nM or less, or 200 nM or less, or 100 nM or less, or 75 nM or less, or 50 nM or less, or 25 nM or less, or 10 nM or less, or 5 nM or less. Avidity can be measured using art-known techniques, such as ELISA or BIACORE.

The antibody or antigen binding fragment of the invention can be produced by any suitable technique, for example, using any suitable eukaryotic or non-eukaryotic expression system. In certain embodiments, the antibody is produced using a mammalian expression system. Alternatively, the antibody or antigen binding fragment of the invention can be produced using a suitable non-eukaryotic expression system such as a bacterial expression system. Bacterial expression systems can be used to produce fragments such as a F (ab) 2, Fv, scFv, F(ab′) 2, F (ab), VL, VH, VHH, dsFv, Fv, scFv-Fc, (scFv) 2, and diabodies.

The antibody or antigen binding fragment of the invention can be conjugated to a synthetic molecule. Conjugation of the antibody or antigen binding fragment of the invention to the synthetic molecule may be by any suitable method, for example recombinant engineering. The synthetic molecule can be any molecule such as a drug targeting a tumour or tumour cells. The synthetic molecule can also be a peptide/protein or an antibody or an antigen binding fragment such that the resultant molecule has specificity for more than one antigen, wherein the resulting fusion protein can be produced by conventional recombinant protein expression systems and methods.

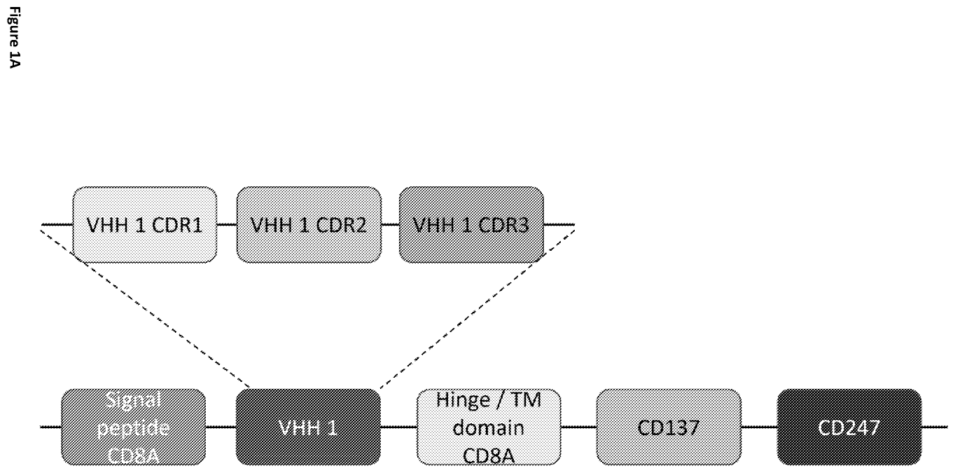

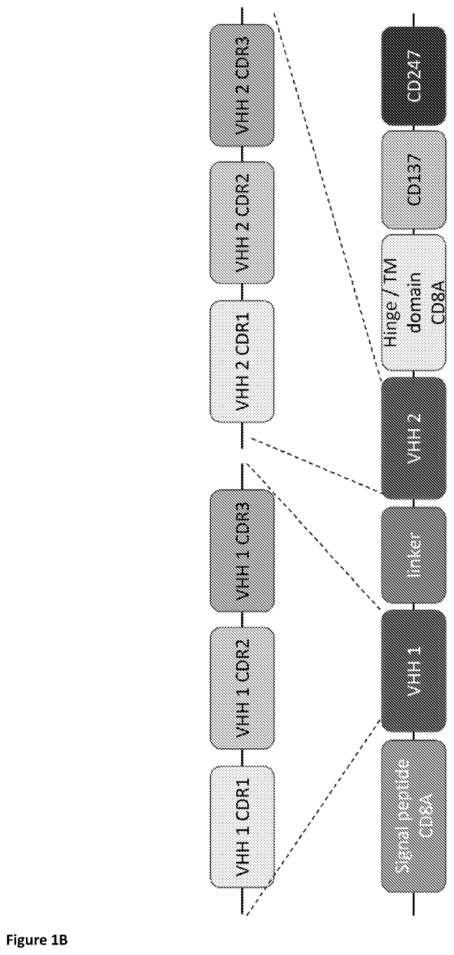

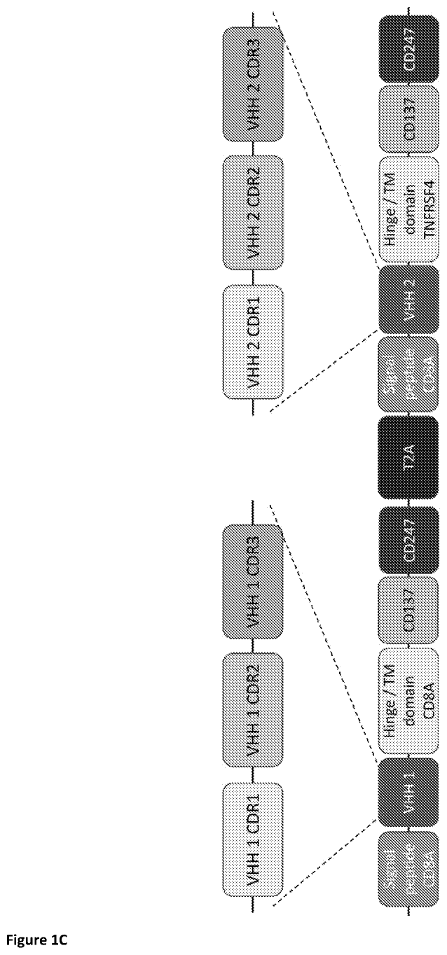

In this respect, a further aspect of the invention relates to chimeric antigen receptors (CARs). CARS, are engineered receptors, which confer specificity for a desired antigen onto an immune effector cell, such as a T-cell. CARs may be expressed on the extracellular surface of a cell. Expression may be by any suitable means via retroviral vector expression. The most common form of these molecules are fusions of an antibody or antigen binding fragment, such as an scFv or VHH, to the transmembrane and intracellular domains of a native T-cell activation receptor, such as CD3, typically CD3-zeta. Such molecules result in the transmission of a signal, suitably an activation signal, in response to recognition by the scFv or VHH of its target. ‘First-generation’ CARs typically have the intracellular domain from the CD3-zeta chain, which is the primary transmitter of signals from endogenous TCRs. ‘Second-generation’ CARs add intracellular signaling domains from various costimulatory protein receptors (e.g., CD28, CD137, 41BB, ICOS) to the intracellular portion of the CAR to provide additional signals to the T cell (see FIG. 1). Preclinical studies have indicated that the second generation of CAR designs improves the antitumor activity of T cells (Maher, et al. (2002) Nat. Biotechnol. 20:70-75; Kowolik, et al. (2006) Cancer Res. 66:10995-11004). ‘Third-generation’ CARs combine multiple signaling domains, such as CD3z-CD28-41BB or CD3z-CD28-OX40, to further augment potency (Zhao, et al. (2009) J. Immunol. 183:5563-5574; Pule, et al. (2005) Mol. Ther. 12:933-941; Zhong, et al. (2010) Mol. Ther. 18:413-420). Accordingly, in an embodiment of this invention, a CAR is provided that comprises an anti-CLL1 and/or an anti-CD33 antibody or antigen binding fragment, such as a VHH fragment, as defined in SEQ ID NOS 5 to 7 and 17 to 18.

CARs of this invention can be prepared using standard recombinant protein techniques using sequences of CD3, e.g. CD247 (CD3-zeta) and optionally other costimulatory molecules known in the art. For example, the human CD247 (CD3-zeta) sequence is available under GENBANK accession number NP_932170, the human CD28 sequence is available under GENBANK accession number NP 006130, the human CD8A sequence is available under GENBANK accession number AAH25715, and the human CD137 sequence is available under GENBANK accession number NP_001552.

In embodiments, the CARs of the present invention include, but are not necessarily limited to: a CD8A extracellular signal peptide (SEQ ID NO: 39); one or more antigen binding fragments of the present invention (SEQ ID NOS: 5, 6 or 7 and 17 or 18) optionally comprising a linker (SEQ ID NOS: 36 to 38) between the one or more antigen binding fragment VHH sequences; a human CD8A hinge/transmembrane domain (SEQ ID NO: 34) or a TNFRSF4 hinge/transmembrane domain (SEQ ID NO: 35); a human CD137 co-stimulatory domain (SEQ ID NO: 33); and a human CD247 (CD3-zeta) intracellular signaling domain (SEQ ID NO: 32) . . . . In particular embodiments, the CARs of the present invention are represented by the SEQ ID NOS: 25 to 31, 40 to 42, 93 and 94.

In specific embodiments, the CARs of the present invention have a single CLL1 or CD33 VHH antigen binding domain, with a protein sequence as defined in SEQ ID NOS: 25 to 31.

In specific embodiments, the CARs as defined in SEQ ID NOS: 25 to 31 are encoded by DNA sequences represented by the SEQ ID NOS: 73 to 79.

In specific embodiments, the CARs of the present invention have tandem CLL1/CD33 VHH antigen binding domains, with protein sequences as defined in SEQ ID NOS: 40 to 42, 93 and 94.

In specific embodiments, the CARs as defined in SEQ ID NOS: 40 to 42, 93 and 94 are encoded by DNA sequences represented by the SEQ ID NOS: 80 to 82, 97 to 99.

In particular embodiments, the CARs of the present invention are represented by SEQ ID NOS: 44 to 46, 95 and 96. These parallel CARs may encompass one or more CARs as defined elsewhere herein (SEQ ID NOS: 25 to 31) linked together by one or more self-cleaving peptides, for example T2A (SEQ ID NO: 43), or they may be different CARS. As defined herein, a CAR sequence comprising one or more full CAR sequences is also referred to as a CAR.

In specific embodiments, the CARs as defined in SEQ ID NOS: 44 to 46, 95 and 96 are encoded by DNA sequences represented by the SEQ ID NOS: 83 to 85, 100 and 101.

In some embodiments, the antibody or the antigen binding fragment can be conjugated to a synthetic molecule that can confer specificity for one or more antigens in addition to CLL1 and/or CD33. For example, the antibody of the invention can be engineered (e.g. as a bivalent diabody or a conjugated Fab dimer or trimer) to have specificity for CLL1 and/or CD33 and another tumour antigen, e.g., an antigen associated with a disease or disorder as disclosed herein. Alternatively, the antibody can be engineered to have specificity for CLL1 and/or CD33 and an antigen that promotes activation or targeting of other cells, such as cytotoxic effector cells or T cells. Accordingly, the invention also includes bispecific, tri-specific and multi-specific molecules such as BiTEs (bi-specific T-cell engagers) and DART™s (dual affinity retargeting reagents).

As is known in the art, a bi-specific T-cell engager (BiTE) refers to a single polypeptide chain molecule having two antigen binding domains, one of which binds to a T-cell antigen (e.g., CD3) and the second of which binds to an antigen present on the surface of a target cell (WO 05/061547; Baeuerle, et al. (2008) Drugs of the Future 33:137-147; Bargou, et al. (2008) Science 321:974-977). BiTE antibodies have been constructed to various target antigens including CD19, EpCAM, Her2/neu, EGFR, CD66e (or CEA, CEACAM5), CD33, EphA2, and MCSP (or HMW-MAA) (Baeuerle, et al. (2009) Curr. Opin. Mol. Ther. 11:22-30). Accordingly, in another embodiment of this invention, an anti-CLL1 and/or anti-CD33 antibody or antigen binding fragment (e.g. a scFv or VHH) is a component of a bi-specific T-cell engager (BiTE). In particular embodiments, the bi-specific T-cell engager (BiTE) of this invention is composed of an anti-CLL1 and/or anti-CD33 antibody or antigen binding fragment and an anti-CD3 antibody fragment fused together by a linker, e.g., a flexible protein linker. See, for example, U.S. Pat. No. 5,929,212. The term ‘BiTE’ as used herein may encompass but is not limited to the bispecific T-cell engager named BiTE® available from Amgen®.

In embodiments, the antibody fragment that specifically binds CD3 comprises:
(a) a heavy chain variable region comprising,
   (i) a CDR1 of SEQ ID NO:67,
   (ii) a CDR2 of SEQ ID NO:68, and
   (iii) a CDR3 of SEQ ID NO:69; and
(b) a light chain variable region comprising,
   (i) a CDR1 of SEQ ID NO:70,
   (ii) a CDR2 of SEQ ID NO:71, and
   (iii) a CDR3 of SEQ ID NO:72.

In other embodiments, the invention provides the antibody or the antigen binding fragment of the invention coupled to a synthetic molecule as defined herein to provide a DART™. DART™ refers to an immunoglobulin molecule that includes at least two polypeptide chains that associate (especially through a covalent interaction) to form at least two antigen binding sites, which may recognize the same or different antigens. Each of the polypeptide chains of a DART™ include a VHH heavy chain region, or an immunoglobulin light chain variable region and an immunoglobulin heavy chain variable region, but these regions do not interact to form an epitope binding site. Rather, the immunoglobulin heavy chain variable region of one (e.g., the first) of the DART™ polypeptide chains interacts with the immunoglobulin light chain variable region of a different (e.g. the second) DART™ polypeptide chain to form an epitope binding site. Similarly, the immunoglobulin light chain variable region of one (e.g. the first) of the DART™ polypeptide chains interacts with the immunoglobulin heavy chain variable region of a different (e.g., the second) DART™ polypeptide chain to form an epitope binding site. DART™s may be monospecific, bi-specific, tri-specific, etc., thus being able to simultaneously bind one, two, three or more different antigens (which may be of the same or of different antigens). DART™s may additionally be monovalent, bivalent, trivalent, tetravalent, pentavalent, hexavalent, etc., thus being able to simultaneously bind one, two, three, four, five, six or more molecules. These two attributes of DART™s (i.e., degree of specificity and valency may be combined, for example to produce bispecific antibodies (i.e., capable of binding two epitopes) that are tetravalent (i.e. capable of binding four sets of antigens/epitopes), etc. The construction of DART™ molecules is disclosed in WO 2006/113665, WO 2008/157379, and WO 2010/080538. Accordingly, in another embodiment of this invention, an anti-CLL1 and/or anti-CD33 antibody or antigen binding fragment is included in a DART™.

In other embodiments, the antibody or antigen binding fragment of the present invention is conjugated, linked or joined to other synthetic molecules including therapeutic agents (or ‘payloads’) such as cytotoxic, cytostatic, or anti-angiogenic agents and radioisotopes, labels or nanoparticles. Such molecules are terms antibody-drug conjugates or ADCs. Examples of ADCs include: Gemtuzumab ozogamicin, Brentuximab vedotin, Trastuzumab emtansine, Trastuzumab emtansine and Trastuzumab emtansine.

The invention provides a method of inhibiting or killing cells that express CLL1 and/or CD33 (CLL1 and/or CD33 cells) by contacting the cells with an antibody, antigen binding fragment or antigen binding molecule as described herein. This includes antibodies or antigen-binding fragments conjugated to a synthetic molecule (e.g. a CAR, a bispecific T-cell engager such as a BiTE, DART™, or ADC) of the invention. The method can be used to inhibit CLL1 and/or CD33 cells in vitro or in a subject (i.e., in vivo). The contacted CLL1 and/or CD33 cells can be in, for example, a cell culture or animal model of a disorder associated with aberrant expression or levels of CLL1 and/or CD33. The method is useful, for example, to measure and/or rank (relative to another antibody) the antibody's inhibitory activity for a CLL1 and/or CD33 cell type. Inhibiting CLL1 and/or CD33 cells can include blocking or reducing the activity or growth of CLL1 and/or CD33 cells. Inhibiting can also include the killing of CLL1 and/or CD33 cells. Cytotoxicity of an antibody, antibody fragment or antibody conjugated to a synthetic molecule such as a BiTE, DART™, or ADC of the invention can be assessed using any conventional assay including, e.g. a lactate dehydrogenase cytotoxicity assay such as the CYTOTOX 96 non-radioactive cytotoxicity assay commercially available from PROMEGA. Similarly, the invention provides an antibody, antigen binding fragment or antibody conjugated to a synthetic molecule (e.g. a bispecific T-cell engager such as a BiTE, DART™, or ADC) of the invention for use in inhibiting and killing cells expressing CLL1 and/or CD33.

The invention also provides a method of treating a subject that has, is suspected to have, or is at risk of a disorder associated with elevated levels of expression of CLL1 and/or CD33 on a cell or elevated levels of cells expressing CLL1 and/or CD33 cells. As used in the context of the present invention, the term ‘elevated’ is intended to include increased CLL1 and/or CD33 expression on a cell as compared to expression of CLL1 and/or CD33 in normal or healthy cells, or an increase in cells expressing CLL1 and/or CD33 compared to a normal or healthy state. Generally, the method of treatment includes administering a therapeutically effective amount of an isolated antibody, antibody fragment or fusion protein of the invention to the subject. The antibody can be any anti-CLL1 and/or anti-CD33 antibody, antigen binding fragment (e.g. chimeric, humanised, synthetic, F (ab) 2, Fv, scFv, F(ab′) 2, F (ab), VL, VH, VHH, dsFv, Fv, or (scFv) 2) or antibody conjugated to a synthetic molecule (e.g., a bispecific T-cell engager such as a BiTE, DART™, or ADC) as described herein. Similarly, the invention provides an antibody, antigen binding fragment or antibody conjugated to a synthetic molecule (e.g., a bispecific T-cell engager such as a BiTE, DART™, or ADC) of the invention for use in medicine, and for the treatment of disease and disorders associated with aberrant or elevated CLL1 and/or CD33 expression.

Disorders that can be treated include, for example, cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

The invention also provides a method of treating a subject that has, is suspected to have, or is at risk of a disorder associated with elevated levels of expression of CLL1 and/or CD33 on a cell, or by elevated levels of cells expressing CLL1 and/or CD33 wherein treatment comprises adoptive transfer of the recombinant host cells, e.g. T-cells described herein, which express an antibody or antigen binding fragment conjugated to a synthetic molecule of the invention as a CAR that selectively binds CLL1 and/or CD33. Similarly, the invention provides recombinant cells expressing an antibody or antigen binding fragment conjugated to a synthetic molecule or antigen binding molecule (e.g. CAR) of the invention for use in medicine, or for use in the manufacture of a medicament.

Recombinant technology can be used to introduce CAR-encoding genetic material into any suitable T-cells, e.g. effector memory T-cells from the subject to be treated. The recombinant T-cells are transferred, typically by infusion, to the patient. The transferred T-cells of the invention can then mount an immune response against CLL1 and/or CD33 expressing cells (CLL1 and/or CD33 cells) in the patient.

The adoptive transfer method can be used, for example, to treat subjects that have or are suspected to have cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

In embodiments, and suitable for use in treatment, the invention also provides a pharmaceutical composition containing an antibody or antigen binding fragment or antibody conjugated to a synthetic molecule or antigen binding molecule (e.g., a CAR, a bispecific T-cell engager such as a BiTE, DART™, or ADC) as described herein and a pharmaceutically acceptable carrier. Pharmaceutical compositions can be prepared from any of the antibody, antigen binding fragment (e.g. chimeric, humanised, synthetic, F (ab) 2, Fv, scFv, F(ab′) 2, F (ab), VL, VH, VHH, dsFv, Fv, or (scFv) 2) or antibody conjugated to a synthetic molecule or antigen binding molecule (e.g., a CAR, a bispecific T-cell engager such as a BiTE, DART™, or ADC) as described herein.

The composition of the invention can include a carrier for the antibody or antigen binding fragment, or antibody conjugated to a synthetic molecule or antigen binding molecule, desirably a pharmaceutically acceptable carrier. The pharmaceutically acceptable carrier can be any suitable pharmaceutically acceptable carrier. The term ‘pharmaceutically acceptable carrier’, as used herein, means one or more compatible solid or liquid fillers, diluents, other excipients, or encapsulating substances, which are suitable for administration into a human or veterinary patient (e.g. a physiologically acceptable carrier or a pharmacologically acceptable carrier). The term ‘carrier’ denotes an organic or inorganic ingredient, natural or synthetic, with which the active ingredient is combined to facilitate the application. The pharmaceutically acceptable carrier can be co-mingled with one or more of the active components, e.g., a hybrid molecule, and with each other, when more than one pharmaceutically acceptable carrier is present in the composition in a manner so as not to substantially impair the desired pharmaceutical efficacy. ‘Pharmaceutically acceptable’ materials typically are capable of administration to a patient without the production of significant undesirable physiological effects such as nausea, dizziness, rash, or gastric upset.

The pharmaceutical composition can contain suitable buffering agents, including, for example, acetic acid in a salt, citric acid in a salt, boric acid in a salt, and phosphoric acid in a salt. The pharmaceutical compositions also optionally can contain suitable preservatives, such as benzalkonium chloride, chlorobutanol, parabens, and thimerosal.

The pharmaceutical composition can be presented in unit dosage form and can be prepared by any suitable method, many of which are well-known in the pharmaceutical arts. Such methods include the step of bringing the antibody of the invention into association with a carrier that constitutes one or more accessory ingredients. In general, the composition is prepared by uniformly and intimately bringing the active agent into association with a liquid carrier, a finely divided solid carrier, or both, and then, if necessary, shaping the product.

### High Throughput Screening to Identify CARs/Antibodies

The present invention benefits from the high-throughput screening methodology as described in the co-pending International application no. PCT/GB2022/050158, the content of which is incorporated herein by reference.

The high-throughput screening method allows resources to be focused primarily on the diversity of the CAR library and identification of the CARs that are able to demonstrate promising activity in a functional assay relevant to a clinical context.

The invention is described in greater detail by the following non-limiting examples.

### EXAMPLES

### Example 1

A synthetic human single domain heavy chain (VHH) phage library was panned against recombinant human CLL1 protein (R&D Biosystems™) and human CD33 protein (R&D Biosystems™). To this end, each library was grown to log phase, and then rescued with M13KO7 helper phage (Antibody Design Lab™, PH010L) before being amplified overnight at 32° C. in a shaker. Each phage library was subsequently precipitated with PEG/NaCl, re-suspended in PBS and stored at −80° C. Protein G coated magnetic beads were coated with hCLL1-Fc or hCD33-Fc or Fc recombinant protein in PBS and subsequently blocked in PBS+ BSA. Phage particles were incubated for 30 minutes with negative magnetic particles (Fc protein). Subsequently, magnetic particles were pelleted and the supernatant was incubated for 1 h with hCLL1 coated magnetic beads or with hCD33 coated magnetic beads under rotation. After 1 h incubation, unbound and non-specifically bound phages were washed away by rinsing the beads with PBST. Bound phages were eluted by 100 mM triethylamine (TEA), and the eluate was neutralized by 1 M Tris-HCl (pH 7.4). Each eluate was then used to infect exponentially growing E. coli TGI cells. The panning was repeated for an additional two to four cycles.

### Example 2

After the last panning step, VHH sequences were PCR amplified from the eluted phages or the isolated plasmids using Q5 DNA polymerase (NEB) and VHH-specific forward and reverse primers. Forward and reverse primers contained overhang sequences, including protein linker sequences and Esp31 restriction sites. Resulting amplicons were cleaned by PCR clean-up and PCR amplicons were directly used as inserts for golden gate assembly. A backbone, a CAR scaffold library, consisting of pooled plasmids containing different CAR scaffolds with variation in VHH domain, hinge domain, transmembrane domain and intracellular signalling domains, was used. Golden gate assembly was performed with NEB Golden Gate kit and incubated, cycling between 16 C and 42 C for 40× cycles. The resulting CAR-library was electroporated into electrocompetent bacteria and grown overnight at 30° C., followed by plasmid maxi-prep isolation. The CAR sequences from the resulting plasmid library were briefly PCR amplified and sequenced on an Oxford Nanopore Technology™ MinION™, using the amplicon sequencing kit.

### Example 3

In order to assess if the CAR library was functionally expressed and recognizing CLL1 and/or CD33, the CAR library was used to produce lentiviral vector particles and was then transduced into human T cells or Jurkat cell line. The resulting CAR-T cell library was expanded for six more days after transduction and assessed for CLL1 and/or CD33 binding. CLL1/CD33 CAR-T cell library cells were stained with CLL1-Fc fusion protein, CD33-Fc fusion protein, or a negative control protein (recombinant Fc, R&D systems). In a second step, cells were stained with PE-conjugated anti-Fc and APC-conjugated anti-CD34 antibody to detect transduced cells. The expression of CARs on the cell surface was assessed.

### Example 4

A CAR-T cell library was produced by activation of PBMCs with TransAct™ (Miltenyi™) in the presence of human IL-2 (100 IU/ml) and transduced at day two with the lentiviral CLL1/CD33 CAR library. The next day, cells were washed and further expanded until day 8 of the process. Transduction was assessed by flow cytometry and transduced cells were enriched through CD34 microbeads (Miltenyi™). Purity was assessed by flow cytometry. The CAR-T-cell library was co-cultured with CLL1/CD33-positive cells (HL60) or no cells. After 6 h activation, CAR-T cells were prepared for single cell sequencing analysis. CAR-T cell libraries from different donors were prepared for 10× genomics single cell gene expression analysis (10× genomics 3′ sequencing kit V3 or Single Cell 5′ Kit v2) and sequenced on a NovaSeq™ 6000 (Illumina™). Cell Ranger™ software (10× Genomics™) was used for downstream processing and alignment of reads. In order to deconvolute the single cell sequencing data and identify the CAR sequence and cell barcode identity, CAR sequences were amplified using long PCR with a forward read 1 primer and CAR-specific reverse primer. After 10 cycles of PCR, the product was cleaned using SPRI bead clean-up and a second, nested PCR was performed. PCR products were barcoded and an Oxford Nanopore library was prepared using the Oxford Nanopore ligation sequencing kit. Libraries were then sequenced on a MinION® flow cell and CAR sequences and 10× cell barcodes were determined.

### Example 5

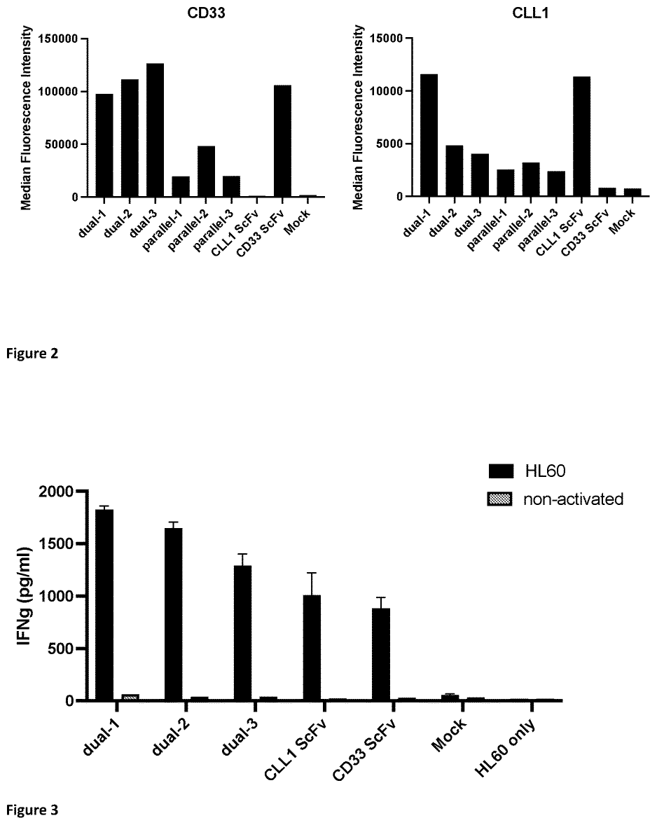

Single CAR constructs were selected based on best activation propensity from the single cell gene expression analysis, and CAR sequences were synthetized by Integrated DNA technologies (IDT). Primary T cells expressing the different CAR constructs were generated and used for functional assays. The affinity/avidity with which the different CARS bind to CLL1 and/or CD33 was assessed. Cells transduced with the different CAR constructs were stained with CLL1-Fc, CD33-Fc or both fusion proteins (R&D systems). In a second step, cells were stained with PE-conjugated anti-Fc and an APC-conjugated anti-CD34 antibodies. Median Fluorescence Intensity deriving from CLL1 and/or CD33 binding was assessed (FIGS. 2 and 6).

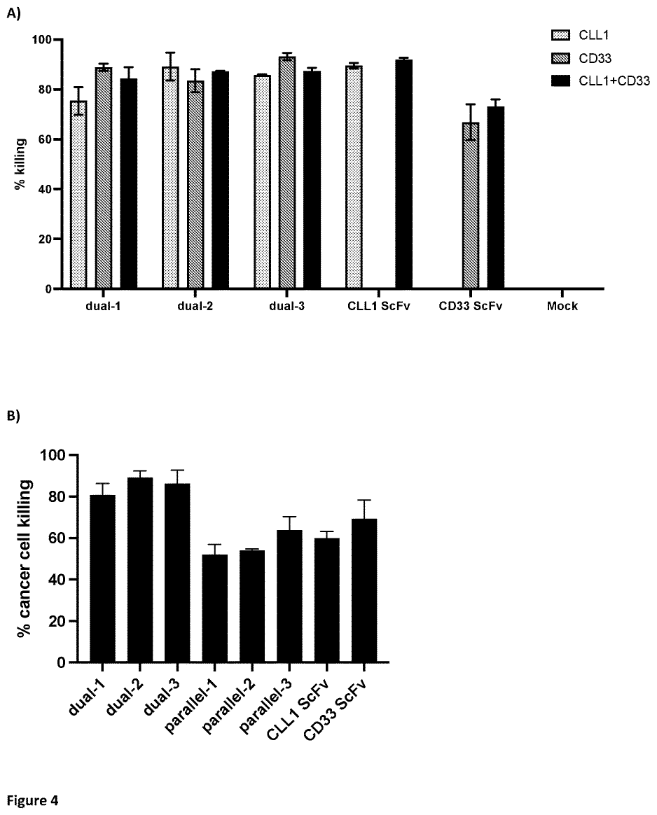

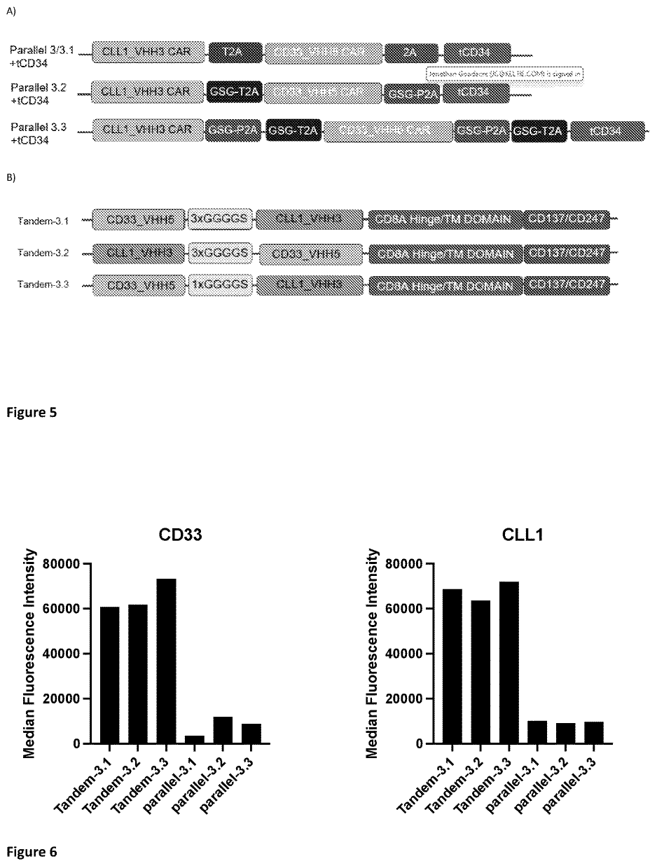

CARs labelled dual-1 to dual-3 in FIGS. 2, 3, 4A and 4B relate to CARs labelled Tandem-1 to Tandem-3 in Table 4 (SEQ ID Nos: 40 to 42 respectively). CARs labelled parallel-1 to parallel-3 in FIGS. 2 and 4B, relate to CARs labelled Parallel-1 to Parallel-3 in Table 5 (SEQ ID Nos: 44 to 46 respectively). The CAR labelled Parallel-3.1 in FIGS. 6, 7, 8 and 9 is identical to the CAR named parallel-3 elsewhere herein.

CARs labelled Tandem-3.1 to Tandem-3.3 in FIGS. 6, 7, 8 and 9 relate to CARs labelled Tandem-3.1 to Tandem-3.3 in Table 4 (SEQ ID Nos: 42, 93 and 94 respectively).

CARs labelled parallel-3.2 and parallel-3.3 in FIGS. 6, 7, 8 and 9 relate to CARs labelled Parallel-3.2 to Parallel-3.3 in Table 5 (SEQ ID Nos: 95 and 96 respectively).

CARs labelled CLL1 scFv and CD33 scFv in FIGS. 2, 3, 4A, 4B, 7, 8 and 9 are comparative examples. CLL1 scFv relates to a comparative example of prior art CLL1 CAR (comparative antibody no. 6), CLL1 scFv relates to comparative example of prior art CD33 CAR (comparative antibody no. 7) ScFv CARS (SEQ ID NOS: 65 to 66) from US2020/0115457A1 and US 2021/0017277 A1 respectively (Table 7). The CARs as defined in SEQ ID NOS: 65 to 66 are encoded by DNA sequences represented by the SEQ ID NOS: 86 to 87.

TABLE 7
Comparative example antibodies from US2020/0115457A1 (comparative antibody no.
6) and US 2021/0017277 A1 (comparative antibody no. 7)

| Comparative | | | | | | | | | |
|---|---|---|---|---|---|---|---|---|---|
| Antibody | | | V_{H} | | | | V_{L} | | |
| No. | scFv. | V_{H} | CDR1 | CDR2 | CDR3 | V_{L} | CDR1 | CDR2 | CDR3 |
| 6 | SEQ ID | SEQ ID | SEQ ID | SEQ ID | SEQ ID | SEQ ID | SEQ ID | SEQ ID | SEQ ID |
| | NO. 47 | NO. 49 | NO. 51: | NO. 53: | NO. 55: | NO. 57 | NO. 59: | NO. 61: | NO. 63: |
| | | | GGSISS | IYYSGST | VSLVYC | | QDINNF | DAS | QQYGNL |
| | | | YY | | GGDCY | | | | PFT |
| | | | | | SGFDY | | | | |
| 7 | SEQ ID | SEQ ID | SEQ ID | SEQ ID | SEQ ID | SEQ ID | SEQ ID | SEQ ID | SEQ ID |
| | NO. 48 | NO. 50 | NO. 52: | NO. 54: | NO. 56: | NO. 58 | NO. 60: | NO. 62: | NO. 64: |
| | | | GYTFT | IYPYNGG | ARGRP | | ESVDN | AAS | QQSKEV |
| | | | DYN | T | AMDY | | YGISF | | PWT |

As is evident from FIG. 2, each of the dual/tandem CARs (SEQ ID NOS 40 to 42) showed similar binding activity against CLL1 and/or CD33 compared to the prior art CARs. The parallel CARs (SEQ ID NOS 44 to 46) still showed comparable activity compared to the prior art CARs.

As is evident from FIG. 6, each of the dual/tandem CARs (SEQ ID NOS 42, 93 and 94) showed similar binding activity against CLL1 or CD33 compared to the CAR labelled Parallel 3.1 (identical to the CAR labelled Parallel-3 in FIGS. 2, 3, 4A and 4B). The parallel CARs (SEQ ID NOS 95 and 96) still showed comparable activity compared to the CAR labelled Parallel 3.1 (identical to the CAR labelled Parallel-3 in FIGS. 2, 3, 4A and 4B).

This demonstrates the surprisingly beneficial effect of the antibody/antigen binding fragments of the present invention, and the utility of such antibody/antigen binding fragments in CARs, other bi-specific, tri-specific and multi-specific molecules (such as BiTEs and DART™s), and ADCs.

### Example 6

To further evaluate the activity of different anti-CLL1/CD33 CARS, CLL1/CD33-CAR T-cells were evaluated for cellular toxicity and activity against cancer cells.

The ability of the different CARs to elicit functional responses was explored by incubating the CAR T-cells for 24 hours at 1:1 ratio with CLL1 and CD33 co-expressing cancer cell line (HL60). After 24 h co-culture, cells were stained with anti-CD3 Pacific Blue, anti-CLL1 APC in order to separate T cells and cancer cells. Cells were acquired and counted using flow cytometry and cell killing was calculated compared to HL60 cells cultured in the absence of CAR-T cells.

In order to assess killing against either target separately, HeLa cell lines were generated, expressing either CD33 alone, CLL1 alone or CD33 and CLL1. CAR-T cells of one aspect of the present invention were co-cultured with Hela cells expressing either CLL1, CD33 or both. After 24 h, Hela cells were quantified using ONE-Glo reagent (Promega) in a luciferase assay (FIG. 4A). This showed that dual-specific (tandem) CAR-T cells, comprising CARs with sequences as defined in SEQ ID NOS 40 to 42, can efficiently kill cancer cells expressing CLL1 and/or CD33 and are still effective in case of antigen-loss.

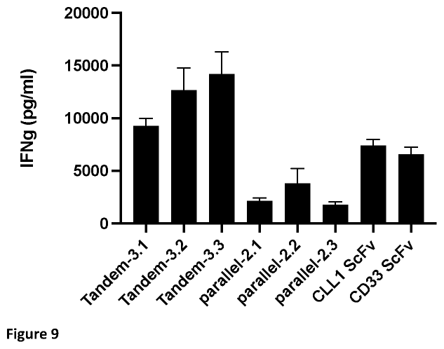

In order to assess killing of the AML suspension cell line HL60, CAR-T cells were co-cultured at 1:1 ratio with HL60 cells, expressing a luciferase reporter. After 24 h, cell killing was quantified via ONE-Glo reagent (Promega) and normalized to HL60 cells cultured in the absence of CAR-T cells. Results for % cancer cell killing is shown for CARS labelled dual-1 to dual-3 (Tandem-1 to Tandem 3) (SEQ ID NOS 40-42) and Parallel-3 to Parallel-3 (SEQ ID NOS 44 to 46) in FIG. 4B; and for CARS labelled Tandem-3.1 to Tandem 3.2 (SEQ ID NOS 42, 93 and 94) and Parallel-3.2 and Parallel-3.3 (SEQ ID NOS 95 and 96) in FIG. 8.

CAR-T cell activation was assessed via upregulation of activation markers, such as CD69 and CD25 on CD3+, CD34+ CAR-T cells after a 24 h co-culture with HL60 cells. As a non-activated control, CAR-T cells were cultured in the absence of target cells for 24 h. After 24 h cells were assessed for activation markers via flow cytometry and the supernatant of the cell co-cultures was used for IFNg ELISA assay. IFNg was quantified using human IFN-gamma DuoSet ELISA (R&D systems) (FIG. 3 and FIG. 9).

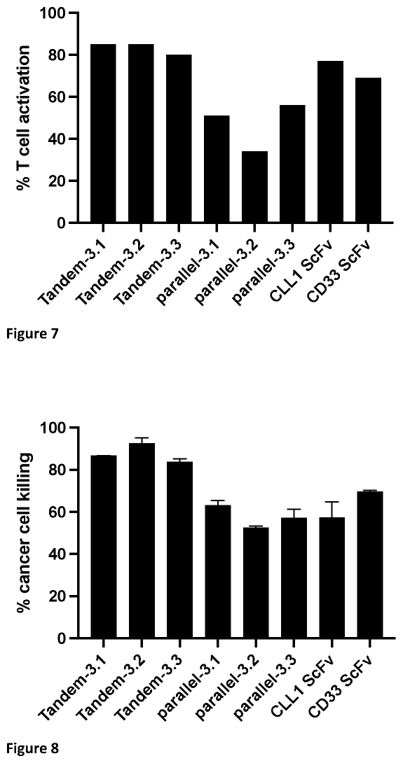

Cells were stimulated for 24 h with target cells and subsequently stained with anti-CD34, anti-CD25 and anti-CD69 fluorescence conjugated antibodies. Activation was assessed in CD34+ cells as the percentage of CD25/CD69 double-positive cells (FIG. 7).

As is evident from the above examples, each of the CARs corresponding to CAR SEQ ID NOs 40 to 42 and 93 to 96, comprising the VHHs SEQ ID NOs 5 to 7 and 17 to 18 (and the associated CDRs, as detailed in Tables 1 and 2) showed enhanced activity against CLL1+ and CD33+ cells than the prior art CARs. This demonstrates the surprisingly beneficial effect of the antibody/antigen binding fragments of the present invention, and the utility of such antibody/antigen binding fragments in CARs, other bi-specific, tri-specific and multi-specific molecules (such as BiTEs and DART™s), and ADCs.

The IFNg response is best in tandem-3.3 and activation was similar in six tandem CARs identified. That said, all CARs tested work well and, perhaps most importantly, all retain the significant advantage that the CD33/CLL1 dual (tandem) and parallel CARs can kill cancer cells that might lose CLL1 or CD33 through epitope escape leading to patient relapse. These CARs therefore represent a significant advancement in the search for effective therapies.

Although particular embodiments of the invention have been disclosed herein in detail, this has been done by way of example and for the purposes of illustration only. The aforementioned embodiments are not intended to be limiting with respect to the scope of the invention. It is contemplated by the inventors that various substitutions, alterations, and modifications may be made to the invention without departing from the spirit and scope of the invention.

Sequence listing:

| SEQ | | |
|---|---|---|
| ID | | |
| NO: | Description | Sequence |
| 1 | CLL1 amino acid | MSEEVTYADLQFQNSSEMEKIPEIGKFGEKAPPAPSHVWRPAALFLTLLCLL |
| | | LLIGLGVLASMFHVTLKIEMKKMNKLQNISEELQRNISLQLMSNMNISNKIRN |
| | | LSTTLQTIATKLCRELYSKEQEHKCKPCPRRWIWHKDSCYFLSDDVQTWQE |
| | | SKMACAAQNASLLKINNKNALEFIKSQSRSYDYWLGLSPEEDSTRGMRVDN |
| | | IINSSAWVIRNAPDLNNMYCGYINRLYVQYYHCTYKKRMICEKMANPVQLG |
| | | STYFREA |
| 2 | CLL1 nucleic acid | ATGTCTGAAGAAGTTACTTATGCAGATCTTCAATTCCAGAACTCCAGTGA |
| | | GATGGAAAAAATCCCAGAAATTGGCAAATTTGGGGAAAAAGCACCTCCA |
| | | GCTCCCTCTCATGTATGGCGTCCAGCAGCCTTGTTTCTGACTCTTCTGT |
| | | GCCTTCTGTTGCTCATTGGATTGGGAGTCTTGGCAAGCATGTTTCACGT |
| | | AACTTTGAAGATAGAAATGAAAAAAATGAACAAACTACAAAACATCAGTG |
| | | AAGAGCTCCAGAGAAATATTTCTCTACAACTGATGAGTAACATGAATATC |
| | | TCCAACAAGATCAGGAACCTCTCCACCACACTGCAAACAATAGCCACCA |
| | | AATTATGTCGTGAGCTATATAGCAAAGAACAAGAGCACAAATGTAAGCCT |
| | | TGTCCAAGGAGATGGATTTGGCATAAGGACAGCTGTTATTTCCTAAGTG |
| | | ATGATGTCCAAACATGGCAGGAGAGTAAAATGGCCTGTGCTGCTCAGAA |
| | | TGCCAGCCTGTTGAAGATAAACAACAAAAATGCATTGGAATTTATAAAAT |
| | | CCCAGAGTAGATCATATGACTATTGGCTGGGATTATCTCCTGAAGAAGA |
| | | TTCCACTCGTGGTATGAGAGTGGATAATATAATCAACTCCTCTGCCTGG |
| | | GTTATAAGAAACGCACCTGACTTAAATAACATGTATTGTGGATATATAAA |
| | | TAGACTATATGTTCAATATTATCACTGCACTTATAAAAAAAGAATGATATG |
| | | TGAGAAGATGGCCAATCCAGTGCAGCTTGGTTCTACATATTTTAGGGAG |
| | | GCATGA |
| 3 | CD33 amino acid | MPLLLLLPLLWAGALAMDPNFWLQVQESVTVQEGLCVLVPCTFFHPIPYYD |
| | | KNSPVHGYWFREGAIISRDSPVATNKLDQEVQEETQGRFRLLGDPSRNNC |
| | | SLSIVDARRRDNGSYFFRMERGSTKYSYKSPQLSVHVTDLTHRPKILIPGTL |
| | | EPGHSKNLTCSVSWACEQGTPPIFSWLSAAPTSLGPRTTHSSVLIITPRPQD |
| | | HGTNLTCQVKFAGAGVTTERTIQLNVTYVPQNPTTGIFPGDGSGKQETRAG |
| | | VVHGAIGGAGVTALLALCLCLIFFIVKTHRRKAARTAVGRNDTHPTTGSASP |
| | | KHQKKSKLHGPTETSSCSGAAPTVEMDEELHYASLNFHGMNPSKDTSTEY |
| | | SEVRTQ |
| 4 | CD33 nucleic acid | ATGCCGCTGCTGCTACTGCTGCCCCTGCTGTGGGCAGGGGCCCTGGCT |
| | | ATGGATCCAAATTTCTGGCTGCAAGTGCAGGAGTCAGTGACGGTACAG |
| | | GAGGGTTTGTGCGTCCTCGTGCCCTGCACTTTCTTCCATCCCATACCCT |
| | | ACTACGACAAGAACTCCCCAGTTCATGGTTACTGGTTCCGGGAAGGAGC |
| | | CATTATATCCAGGGACTCTCCAGTGGCCACAAACAAGCTAGATCAAGAA |
| | | GTACAGGAGGAGACTCAGGGCAGATTCCGCCTCCTTGGGGATCCCAGT |
| | | AGGAACAACTGCTCCCTGAGCATCGTAGACGCCAGGAGGAGGGATAAT |
| | | GGTTCATACTTCTTTCGGATGGAGAGAGGAAGTACCAAATACAGTTACA |
| | | AATCTCCCCAGCTCTCTGTGCATGTGACAGACTTGACCCACAGGCCCAA |
| | | AATCCTCATCCCTGGCACTCTAGAACCCGGCCACTCCAAAAACCTGACC |
| | | TGCTCTGTGTCCTGGGCCTGTGAGCAGGGAACACCCCCGATCTTCTCC |
| | | TGGTTGTCAGCTGCCCCCACCTCCCTGGGCCCCAGGACTACTCACTCC |
| | | TCGGTGCTCATAATCACCCCACGGCCCCAGGACCACGGCACCAACCTG |
| | | ACCTGTCAGGTGAAGTTCGCTGGAGCTGGTGTGACTACGGAGAGAACC |
| | | ATCCAGCTCAACGTCACCTATGTTCCACAGAACCCAACAACTGGTATCTT |
| | | TCCAGGAGATGGCTCAGGGAAACAAGAGACCAGAGCAGGAGTGGTTCA |
| | | TGGGGCCATTGGAGGAGCTGGTGTTACAGCCCTGCTCGCTCTTTGTCTC |
| | | TGCCTCATCTTCTTCATAGTGAAGACCCACAGGAGGAAAGCAGCCAGGA |
| | | CAGCAGTGGGCAGGAATGACACCCACCCTACCACAGGGTCAGCCTCCC |
| | | CGAAACACCAGAAGAAGTCCAAGTTACATGGCCCCACTGAAACCTCAAG |
| | | CTGTTCAGGTGCCGCCCCTACTGTGGAGATGGATGAGGAGCTGCATTA |
| | | TGCTTCCCTCAACTTTCATGGGATGAATCCTTCCAAGGACACCTCCACC |
| | | GAATACTCAGAGGTCAGGACCCAG |
| 5 | VHH of Antibody 1 | EVQLVESGGGLVQPGGSLRLSCAASGFTFSTYDMSWVRQAPGKQREWVS |
| | | AISGSGGTTYYADSVKGRFTISRDNSKNTLYLQMNSLRAEDTAVYYCVRGD |
| | | DHWGQGTLVTVSS |
| 6 | VHH of Antibody 2 | EVQLVESGGGLVQPGGSLRLSCAASGFTFSNYDMSWVRQAPGKQLEWVS |
| | | AITGSGGSTYYADSVKGRFTISRDNSKNTLYLQMNSLRAEDTAVYYCAAGV |
| | | DGDYWGQGTLVTVSS |
| 7 | VHH of Antibody 3 | EVQLVESGGGLVQPGGSLRLSCAASGFTFSAYDMSWVRQAPGKQLEWVS |
| | | AISGSGGSTYYADSVKGRFTISRDNSKNTLYLQMNSLRAEDTAVYYCAYDG |
| | | DSTGSLDEMAHWGQGTLVTVSS |
| 8 | CDR1 of Antibody 1 | GFTFSTYD |
| 9 | CDR1 of Antibody 2 | GFTFSNYD |
| 10 | CDR1 of Antibody 3 | GFTFSAYD |
| 11 | CDR2 of Antibody 1 | ISGSGGTT |
| 12 | CDR2 of Antibody 2 | ITGSGGST |
| 13 | CDR2 of Antibody 3 | ISGSGGST |
| 14 | CDR3 of Antibody 1 | VRGDDH |
| 15 | CDR3 of Antibody 2 | AAGVDGDY |
| 16 | CDR3 of Antibody 3 | AYDGDSTGSLDEMAH |
| 17 | VHH of Antibody 4 | EVOLVESGGGLVQPGGSLRLSCAASGFTFSTYAMSWVRQAPGKQREWVS |
| | | AIEGSGGSTYYADSVKGRFTISRDNSKNTLYLQMNSLRAEDTAVYYCAVET |
| | | GWADFDYWGQGTLVTVSS |
| 18 | VHH of Antibody 5 | EVOLVESGGGLVQPGGSLRLSCAASGFTFSYYAMSWVRQAPGKQREWVS |
| | | AIEGSGGRTYYADSVKGRFTISRDNSKNTLYLQMNSLRAEDTAVYYCVKET |
| | | GWADFDYWGQGTLVTVSS |
| 19 | CDR1 of Antibody 4 | GFTFSTYA |
| 20 | CDR1 of Antibody 5 | GFTFSYYA |
| 21 | CDR2 of Antibody 4 | IEGSGGST |
| 22 | CDR2 of Antibody 5 | IEGSGGRT |
| 23 | CDR3 of Antibody 4 | AVETGWADFDY |
| 24 | CDR3 of Antibody 5 | VKETGWADFDY |
| 25 | CAR comprising Antibody 1 | MALPVTALLLPLALLLHAARPEVQLVESGGGLVQPGGSLRLSCAASGFTFS |
| | Single-1 | TYDMSWVRQAPGKQREWVSAISGSGGTTYYADSVKGRFTISRDNSKNTLY |
| | | LQMNSLRAEDTAVYYCVRGDDHWGQGTLVTVSSTTTPAPRPPTPAPTIAS |
| | | QPLSLRPEACRPAAGGAVHTRGLDFACDIYIWAPLAGTCGVLLLSLVITLYC |
| | | NHRNRRRVCKRGRKKLLYIFKQPFMRPVQTTQEEDGCSCRFPEEEEGGCE |
| | | LRVKFSRSADAPAYQQGQNQLYNELNLGRREEYDVLDKRRGRDPEMGGK |
| | | PQRRKNPQEGLYNELQKDKMAEAYSEIGMKGERRRGKGHDGLYQGLSTA |
| | | TKDTYDALHMQALPPR |
| 26 | CAR comprising Antibody 2 | MALPVTALLLPLALLLHAARPEVQLVESGGGLVQPGGSLRLSCAASGFTFS |
| | Single-2 | NYDMSWVRQAPGKQLEWVSAITGSGGSTYYADSVKGRFTISRDNSKNTLY |
| | | LQMNSLRAEDTAVYYCAAGVDGDYWGQGTLVTVSSTTTPAPRPPTPAPTI |
| | | ASQPLSLRPEACRPAAGGAVHTRGLDFACDIYIWAPLAGTCGVLLLSLVITL |
| | | YCNHRNRRRVCKRGRKKLLYIFKQPFMRPVQTTQEEDGCSCRFPEEEEGG |
| | | CELRVKFSRSADAPAYQQGQNQLYNELNLGRREEYDVLDKRRGRDPEMG |
| | | GKPQRRKNPQEGLYNELQKDKMAEAYSEIGMKGERRRGKGHDGLYQGLS |
| | | TATKDTYDALHMQALPPR |
| 27 | CAR comprising Antibody 3 | MALPVTALLLPLALLLHAARPEVQLVESGGGLVQPGGSLRLSCAASGFTFS |
| | Single-3 | AYDMSWVRQAPGKQLEWVSAISGSGGSTYYADSVKGRFTISRDNSKNTLY |
| | | LQMNSLRAEDTAVYYCAYDGDSTGSLDEMAHWGQGTLVTVSSTTTPAPRP |
| | | PTPAPTIASQPLSLRPEACRPAAGGAVHTRGLDFACDIYIWAPLAGTCGVLL |
| | | LSLVITLYCNHRNRRRVCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPQRRKNPQEGLYNELQKDKMAEAYSEIGMKGERRRGKGHD |
| | | GLYQGLSTATKDTYDALHMQALPPR |
| 28 | CAR comprising Antibody 4 | MALPVTALLLPLALLLHAARPEVQLVESGGGLVQPGGSLRLSCAASGFTFS |
| | Single-4A | TYAMSWVRQAPGKQREWVSAIEGSGGSTYYADSVKGRFTISRDNSKNTLY |
| | | LQMNSLRAEDTAVYYCAVETGWADFDYWGQGTLVTVSSTTTPAPRPPTPA |
| | | PTIASQPLSLRPEACRPAAGGAVHTRGLDFACDIYIWAPLAGTCGVLLLSLVI |
| | | TLYCNHRNRRRVCKRGRKKLLYIFKQPFMRPVQTTQEEDGCSCRFPEEEE |
| | | GGCELRVKFSRSADAPAYQQGQNQLYNELNLGRREEYDVLDKRRGRDPE |
| | | MGGKPQRRKNPQEGLYNELQKDKMAEAYSEIGMKGERRRGKGHDGLYQ |
| | | GLSTATKDTYDALHMQALPPR |
| 29 | CAR comprising Antibody 5 | MALPVTALLLPLALLLHAARPEVQLVESGGGLVQPGGSLRLSCAASGFTFS |
| | Single-5A | YYAMSWVRQAPGKQREWVSAIEGSGGRTYYADSVKGRFTISRDNSKNTLY |
| | | LQMNSLRAEDTAVYYCVKETGWADFDYWGQGTLVTVSSTTTPAPRPPTPA |
| | | PTIASQPLSLRPEACRPAAGGAVHTRGLDFACDIYIWAPLAGTCGVLLLSLVI |
| | | TLYCNHRNRRRVCKRGRKKLLYIFKQPFMRPVQTTQEEDGCSCRFPEEEE |
| | | GGCELRVKFSRSADAPAYQQGQNQLYNELNLGRREEYDVLDKRRGRDPE |
| | | MGGKPQRRKNPQEGLYNELQKDKMAEAYSEIGMKGERRRGKGHDGLYQ |
| | | GLSTATKDTYDALHMQALPPR |
| 30 | CAR comprising Antibody 4 | MALPVTALLLPLALLLHAARPEVQLVESGGGLVQPGGSLRLSCAASGFTFS |
| | and TNFRSF4 hinge/TM | TYAMSWVRQAPGKQREWVSAIEGSGGSTYYADSVKGRFTISRDNSKNTLY |
| | domain | LQMNSLRAEDTAVYYCAVETGWADFDYWGQGTLVTVSSTQPQETQGPPA |
| | Single-4B | RPITVQPTEAWPRTSQGPSTRPVEVPGGRAVAAILGLGLVLGLLGPLAILLA |
| | | LYLLRRDQRKRGRKKLLYIFKQPFMRPVQTTQEEDGCSCRFPEEEEGGCE |
| | | LRVKFSRSADAPAYQQGQNQLYNELNLGRREEYDVLDKRRGRDPEMGGK |
| | | PQRRKNPQEGLYNELQKDKMAEAYSEIGMKGERRRGKGHDGLYQGLSTA |
| | | TKDTYDALHMQALPPR |
| 31 | CAR comprising Antibody 5 | MALPVTALLLPLALLLHAARPEVQLVESGGGLVQPGGSLRLSCAASGFTFS |
| | and TNFRSF4 hinge/TM | YYAMSWVRQAPGKQREWVSAIEGSGGRTYYADSVKGRFTISRDNSKNTLY |
| | domain | LQMNSLRAEDTAVYYCVKETGWADFDYWGQGTLVTVSSTQPQETQGPPA |
| | Single-5B | RPITVQPTEAWPRTSQGPSTRPVEVPGGRAVAAILGLGLVLGLLGPLAILLA |
| | | LYLLRRDQRKRGRKKLLYIFKQPFMRPVQTTQEEDGCSCRFPEEEEGGCE |
| | | LRVKFSRSADAPAYQQGQNQLYNELNLGRREEYDVLDKRRGRDPEMGGK |
| | | PQRRKNPQEGLYNELQKDKMAEAYSEIGMKGERRRGKGHDGLYQGLSTA |
| | | TKDTYDALHMQALPPR |
| 32 | Human CD247 (CD3-zeta) | RVKFSRSADAPAYQQGQNQLYNELNLGRREEYDVLDKRRGRDPEMGGKP |
| | intracellular signaling domain | QRRKNPQEGLYNELQKDKMAEAYSEIGMKGERRRGKGHDGLYQGLSTAT |
| | | KDTYDALHMQALPPR |
| 33 | Human CD137 co-stimulatory | KRGRKKLLYIFKQPFMRPVQTTQEEDGCSCRFPEEEEGGCEL |
| | domain | |
| 34 | Human CD8A | TTTPAPRPPTPAPTIASQPLSLRPEACRPAAGGAVHTRGLDFACDIYIWAPL |
| | hinge/transmembrane domain | AGTCGVLLLSLVITLYCNHRNRRRVC |
| 35 | TNFRSF4 | TQPQETQGPPARPITVQPTEAWPRTSQGPSTRPVEVPGGRAVAAILGLGLV |
| | hinge/transmembrane domain | LGLLGPLAILLALYLLRRDQR |
| 36 | Linker 1 | GGGGSGGGGSGGGGS |
| 37 | Linker 2 | GGGGSGGGGSGGGGSGGGGS |
| 38 | Linker 3 | GGGGS |
| 39 | CD8A extracellular signal | MALPVTALLLPLALLLHAARP |
| | peptide | |
| 40 | Tandem CAR comprising | MALPVTALLLPLALLLHAARPEVQLVESGGGLVQPGGSLRLSCAASGFTFS |
| | Antibodies 1 and 4 | TYDMSWVRQAPGKQREWVSAISGSGGTTYYADSVKGRFTISRDNSKNTLY |
| | Tandem-1 | LQMNSLRAEDTAVYYCVRGDDHWGQGTLVTVSSGGGGSGGGGSGGGGS |
| | | EVOLVESGGGLVQPGGSLRLSCAASGFTFSTYAMSWVRQAPGKQREWVS |
| | | AIEGSGGSTYYADSVKGRFTISRDNSKNTLYLQMNSLRAEDTAVYYCAVET |
| | | GWADFDYWGQGTLVTVSSTTTPAPRPPTPAPTIASQPLSLRPEACRPAAG |
| | | GAVHTRGLDFACDIYIWAPLAGTCGVLLLSLVITLYCNHRNRRRVCKRGRKK |
| | | LLYIFKQPFMRPVQTTQEEDGCSCRFPEEEEGGCELRVKFSRSADAPAYQ |
| | | QGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPP |
| | | R |
| 41 | Tandem CAR comprising | MALPVTALLLPLALLLHAARPEVQLVESGGGLVQPGGSLRLSCAASGFTFS |
| | Antibodies 2 and 4 | NYDMSWVRQAPGKQLEWVSAITGSGGSTYYADSVKGRFTISRDNSKNTLY |
| | Tandem-2 | LQMNSLRAEDTAVYYCAAGVDGDYWGQGTLVTVSSGGGGSGGGGSGGG |
| | | GSEVOLVESGGGLVQPGGSLRLSCAASGFTFSTYAMSWVRQAPGKQREW |
| | | VSAIEGSGGSTYYADSVKGRFTISRDNSKNTLYLQMNSLRAEDTAVYYCAV |
| | | ETGWADFDYWGQGTLVTVSSTTTPAPRPPTPAPTIASQPLSLRPEACRPAA |
| | | GGAVHTRGLDFACDIYIWAPLAGTCGVLLLSLVITLYCNHRNRRRVCKRGR |
| | | KKLLYIFKQPFMRPVQTTQEEDGCSCRFPEEEEGGCELRVKFSRSADAPAY |
| | | QQGQNQLYNELNLGRREEYDVLDKRRGRDPEMGGKPQRRKNPQEGLYN |
| | | ELQKDKMAEAYSEIGMKGERRRGKGHDGLYQGLSTATKDTYDALHMQALP |
| | | PR |
| 42 | Tandem CAR comprising | MALPVTALLLPLALLLHAARPEVQLVESGGGLVQPGGSLRLSCAASGFTFS |
| | Antibodies 5 and 3 | YYAMSWVRQAPGKQREWVSAIEGSGGRTYYADSVKGRFTISRDNSKNTLY |
| | Tandem-3 | LQMNSLRAEDTAVYYCVKETGWADFDYWGQGTLVTVSSGGGGSGGGGS |
| | | GGGGSEVQLVESGGGLVQPGGSLRLSCAASGFTFSAYDMSWVRQAPGK |
| | | QLEWVSAISGSGGSTYYADSVKGRFTISRDNSKNTLYLQMNSLRAEDTAVY |
| | | YCAYDGDSTGSLDEMAHWGQGTLVTVSSTTTPAPRPPTPAPTIASQPLSLR |
| | | PEACRPAAGGAVHTRGLDFACDIYIWAPLAGTCGVLLLSLVITLYCNHRNRR |
| | | RVCKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPQRRK |
| | | NPQEGLYNELQKDKMAEAYSEIGMKGERRRGKGHDGLYQGLSTATKDTY |
| | | DALHMQALPPR |
| 43 | T2A | EGRGSLLTCGDVEENPGP |
| 44 | Parallel CAR comprising | MALPVTALLLPLALLLHAARPEVQLVESGGGLVQPGGSLRLSCAASGFTFS |
| | Antibodies 1 and 4 | TYDMSWVRQAPGKQREWVSAISGSGGTTYYADSVKGRFTISRDNSKNTLY |
| | Parallel-1 | LQMNSLRAEDTAVYYCVRGDDHWGQGTLVTVSSTTTPAPRPPTPAPTIAS |
| | | QPLSLRPEACRPAAGGAVHTRGLDFACDIYIWAPLAGTCGVLLLSLVITLYC |
| | | NHRNRRRVCKRGRKKLLYIFKQPFMRPVQTTQEEDGCSCRFPEEEEGGCE |
| | | LRVKFSRSADAPAYQQGQNQLYNELNLGRREEYDVLDKRRGRDPEMGGK |
| | | PQRRKNPQEGLYNELQKDKMAEAYSEIGMKGERRRGKGHDGLYQGLSTA |
| | | TKDTYDALHMQALPPREGRGSLLTCGDVEENPGPMALPVTALLLPLALLLH |
| | | AARPEVQLVESGGGLVQPGGSLRLSCAASGFTFSTYAMSWVRQAPGKQR |
| | | EWVSAIEGSGGSTYYADSVKGRFTISRDNSKNTLYLQMNSLRAEDTAVYYC |
| | | AVETGWADFDYWGQGTLVTVSSTQPQETQGPPARPITVQPTEAWPRTSQ |
| | | GPSTRPVEVPGGRAVAAILGLGLVLGLLGPLAILLALYLLRRDQRKRGRKKL |
| | | LYIFKQPFMRPVQTTQEEDGCSCRFPEEEEGGCELRVKFSRSADAPAYQQ |
| | | GQNQLYNELNLGRREEYDVLDKRRGRDPEMGGKPQRRKNPQEGLYNELQ |
| | | KDKMAEAYSEIGMKGERRRGKGHDGLYQGLSTATKDTYDALHMQALPPR |
| 45 | Parallel CAR comprising | MALPVTALLLPLALLLHAARPEVQLVESGGGLVQPGGSLRLSCAASGFTFS |
| | Antibodies 2 and 4 | NYDMSWVRQAPGKQLEWVSAITGSGGSTYYADSVKGRFTISRDNSKNTLY |
| | Parallel-2 | LQMNSLRAEDTAVYYCAAGVDGDYWGQGTLVTVSSTTTPAPRPPTPAPTI |
| | | ASQPLSLRPEACRPAAGGAVHTRGLDFACDIYIWAPLAGTCGVLLLSLVITL |
| | | YCNHRNRRRVCKRGRKKLLYIFKQPFMRPVQTTQEEDGCSCRFPEEEEGG |
| | | CELRVKFSRSADAPAYQQGQNQLYNELNLGRREEYDVLDKRRGRDPEMG |
| | | GKPQRRKNPQEGLYNELQKDKMAEAYSEIGMKGERRRGKGHDGLYQGLS |
| | | TATKDTYDALHMQALPPREGRGSLLTCGDVEENPGPMALPVTALLLPLALL |
| | | LHAARPEVQLVESGGGLVQPGGSLRLSCAASGFTFSTYAMSWVRQAPGK |
| | | QREWVSAIEGSGGSTYYADSVKGRFTISRDNSKNTLYLQMNSLRAEDTAVY |
| | | YCAVETGWADFDYWGQGTLVTVSSTQPQETQGPPARPITVQPTEAWPRT |
| | | SQGPSTRPVEVPGGRAVAAILGLGLVLGLLGPLAILLALYLLRRDQRKRGRK |
| | | KLLYIFKQPFMRPVQTTQEEDGCSCRFPEEEEGGCELRVKFSRSADAPAY |
| | | QQGQNQLYNELNLGRREEYDVLDKRRGRDPEMGGKPQRRKNPQEGLYN |
| | | ELQKDKMAEAYSEIGMKGERRRGKGHDGLYQGLSTATKDTYDALHMQALP |
| | | PR |
| 46 | Parallel CAR comprising | MALPVTALLLPLALLLHAARPEVQLVESGGGLVQPGGSLRLSCAASGFTFS |
| | Antibodies 3 and 5 | AYDMSWVRQAPGKQLEWVSAISGSGGSTYYADSVKGRFTISRDNSKNTLY |
| | Parallel-3 | LQMNSLRAEDTAVYYCAYDGDSTGSLDEMAHWGQGTLVTVSSTTTPAPRP |
| | | PTPAPTIASQPLSLRPEACRPAAGGAVHTRGLDFACDIYIWAPLAGTCGVLL |
| | | LSLVITLYCNHRNRRRVCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPQRRKNPQEGLYNELQKDKMAEAYSEIGMKGERRRGKGHD |
| | | GLYQGLSTATKDTYDALHMQALPPREGRGSLLTCGDVEENPGPMALPVTA |
| | | LLLPLALLLHAARPEVQLVESGGGLVQPGGSLRLSCAASGFTFSYYAMSWV |
| | | RQAPGKQREWVSAIEGSGGRTYYADSVKGRFTISRDNSKNTLYLQMNSLR |
| | | AEDTAVYYCVKETGWADFDYWGQGTLVTVSSTQPQETQGPPARPITVQPT |
| | | EAWPRTSQGPSTRPVEVPGGRAVAAILGLGLVLGLLGPLAILLALYLLRRDQ |
| | | RKRGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPQRRKNP |
| | | QEGLYNELQKDKMAEAYSEIGMKGERRRGKGHDGLYQGLSTATKDTYDAL |
| | | HMQALPPR |
| 47 | ScFv of Comparative Antibody | QVQLQESGPGLVKPSETLSLTCTVSGGSISSYYWSWIRQPPGKGLEWIGYI |
| | 6 | YYSGSTNYNPSLKSRVTISVDTSKNQFSLKLSSVTAADTAVYYCVSLVYCG |
| | | GDCYSGFDYWGQGTLVTVSSGGGGSGGGGSGGGGSDIQLTQSPSSLSA |
| | | SVGDRVSFTCQASQDINNFLNWYQQKPGKAPKLLIYDASNLETGVPSRFSG |
| | | SGSGTDFTFTISSLQPEDIATYYCQQYGNLPFTFGGGTKVEIK |
| 48 | ScFv of Comparative Antibody | QVQLVQSGAEVKKPGSSVKVSCKASGYTFTDYNMHWVRQAPGQGLEWIG |
| | 7 | YIYPYNGGTGYNQKFKSKATITADESTNTAYMELSSLRSEDTAVYYCARGR |
| | | PAMDYWGQGTLVTVSSGGGGSGGGGSGGGGSDIQMTQSPSSLSASVGD |
| | | RVTITCRASESVDNYGISFMNWFQQKPGKAPKLLIYAASNQGSGVPSRFSG |
| | | SGSGTDFTLTISSLQPDDFATYYCQQSKEVPWTFGQGTKVEIK |
| 49 | VH of Comparative Antibody 6 | QVQLQESGPGLVKPSETLSLTCTVSGGSISSYYWSWIRQPPGKGLEWIGYI |
| | | YYSGSTNYNPSLKSRVTISVDTSKNQFSLKLSSVTAADTAVYYCVSLVYCG |
| | | GDCYSGFDYWGQGTLVTVSS |
| 50 | VH of Comparative Antibody 7 | QVQLVQSGAEVKKPGSSVKVSCKASGYTFTDYNMHWVRQAPGQGLEWIG |
| | | YIYPYNGGTGYNQKFKSKATITADESTNTAYMELSSLRSEDTAVYYCARGR |
| | | PAMDYWGQGTLVTVSS |
| 51 | CDR1 of VH of Comparative | GGSISSYY |
| | Antibody 6 | |
| 52 | CDR1 of VH of Comparative | GYTFTDYN |
| | Antibody 7 | |
| 53 | CDR2 of VH of Comparative | IYYSGST |
| | Antibody 6 | |
| 54 | CDR2 of VH of Comparative | IYPYNGGT |
| | Antibody 7 | |
| 55 | CDR3 of VH of Comparative | VSLVYCGGDCYSGFDY |
| | Antibody 6 | |
| 56 | CDR3 of VH of Comparative | ARGRPAMDY |
| | Antibody 7 | |
| 57 | VL of Comparative Antibody 6 | DIQLTQSPSSLSASVGDRVSFTCQASQDINNFLNWYQQKPGKAPKLLIYDA |
| | | SNLETGVPSRFSGSGSGTDFTFTISSLQPEDIATYYCQQYGNLPFTFGGGT |
| | | KVEIK |
| 58 | VL of Comparative Antibody 7 | DIQMTQSPSSLSASVGDRVTITCRASESVDNYGISFMNWFQQKPGKAPKLL |
| | | IYAASNQGSGVPSRFSGSGSGTDFTLTISSLQPDDFATYYCQQSKEVPWTF |
| | | GQGTKVEIK |
| 59 | CDR1 of VL of Comparative | QDINNF |
| | Antibody 6 | |
| 60 | CDR1 of VL of Comparative | ESVDNYGISF |
| | Antibody 7 | |
| 61 | CDR2 of VL of Comparative | DAS |
| | Antibody 6 | |
| 62 | CDR2 of VL of Comparative | AAS |
| | Antibody 7 | |
| 63 | CDR3 of VL of Comparative | QQYGNLPFT |
| | Antibody 6 | |
| 64 | CDR3 of VL of Comparative | QQSKEVPWT |
| | Antibody 7 | |
| 65 | CAR comprising Comparative | MALPVTALLLPLALLLHAARPQVQLQESGPGLVKPSETLSLTCTVSGGSISS |
| | Antibody 6 | YYWSWIRQPPGKGLEWIGYIYYSGSTNYNPSLKSRVTISVDTSKNQFSLKLS |
| | | SVTAADTAVYYCVSLVYCGGDCYSGFDYWGQGTLVTVSSGGGGSGGGG |
| | | SGGGGSDIQLTQSPSSLSASVGDRVSFTCQASQDINNFLNWYQQKPGKAP |
| | | KLLIYDASNLETGVPSRFSGSGSGTDFTFTISSLQPEDIATYYCQQYGNLPF |
| | | TFGGGTKVEIKTTTPAPRPPTPAPTIASQPLSLRPEACRPAAGGAVHTRGLD |
| | | FACDIYIWAPLAGTCGVLLLSLVITLYCNHRNRRRVCKRGRKKLLYIFKQPFM |
| | | RPVQTTQEEDGCSCRFPEEEEGGCELRVKFSRSADAPAYQQGQNQLYNE |
| | | LNLGRREEYDVLDKRRGRDPEMGGKPQRRKNPQEGLYNELQKDKMAEAY |
| | | SEIGMKGERRRGKGHDGLYQGLSTATKDTYDALHMQALPPR |
| 66 | CAR comprising Comparative | MALPVTALLLPLALLLHAARPQVQLVQSGAEVKKPGSSVKVSCKASGYTFT |
| | Antibody 7 | DYNMHWVRQAPGQGLEWIGYIYPYNGGTGYNQKFKSKATITADESTNTAY |
| | | MELSSLRSEDTAVYYCARGRPAMDYWGQGTLVTVSSGGGGSGGGGSGG |
| | | GGSDIQMTQSPSSLSASVGDRVTITCRASESVDNYGISFMNWFQQKPGKA |
| | | PKLLIYAASNQGSGVPSRFSGSGSGTDFTLTISSLQPDDFATYYCQQSKEV |
| | | PWTFGQGTKVEIKTTTPAPRPPTPAPTIASQPLSLRPEACRPAAGGAVHTR |
| | | GLDFACDIYIWAPLAGTCGVLLLSLVITLYCNHRNRRRVCKRGRKKLLYIFKQ |
| | | PFMRPVQTTQEEDGCSCRFPEEEEGGCELRVKFSRSADAPAYQQGQNQL |
| | | YNELNLGRREEYDVLDKRRGRDPEMGGKPQRRKNPQEGLYNELQKDKMA |
| | | EAYSEIGMKGERRRGKGHDGLYQGLSTATKDTYDALHMQALPPR |
| 67 | V_{H} CDR1 of antibody fragment | SGYTFTRYTMH |
| | that binds CD3 | |
| 68 | V_{H} CDR2 of antibody fragment | YINPSRGYTNYNQKFKD |
| | that binds CD3 | |
| 69 | V_{H} CDR3 of antibody fragment | YYDDHYCL |
| | that binds CD3 | |
| 70 | V_{L} CDR1 of antibody fragment | SASSSVSYMN |
| | that binds CD3 | |
| 71 | V_{L} CDR2 of antibody fragment | DTSKLAS |
| | that binds CD3 | |
| 72 | V_{L} CDR3 of antibody fragment | QQWSSNPF |
| | that binds CD3 | |
| 73 | CAR comprising Antibody 1 | ATGGCCCTTCCCGTTACCGCCTTACTCTTACCTCTTGCTTTATTACTGCA |
| | nucleic acid | CGCGGCACGGCCTGAAGTCCAACTCGTCGAAAGCGGGGGAGGCTTGG |
| | Single-1 | TACAGCCTGGGGGGTCCCTGAGACTCTCCTGTGCAGCCTCTGGATTCA |
| | | CCTTTAGCACATATGATATGAGCTGGGTCCGCCAGGCTCCAGGGAAGC |
| | | AGCGTGAGTGGGTCTCAGCTATTTCCGGTAGTGGTGGTACAACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGTGCGCGGCGACGACCATTGGGGCCAGGGAACCCT |
| | | CGTGACAGTTAGCAGCACTACCACACCAGCTCCCAGGCCACCCACGCC |
| | | TGCACCGACAATCGCATCTCAACCACTCTCACTCCGCCCCGAAGCGTG |
| | | CAGACCCGCAGCAGGTGGAGCCGTACATACTAGGGGTCTTGACTTCGC |
| | | CTGTGATATATATATTTGGGCGCCCTTGGCCGGGACTTGTGGGGTCCTT |
| | | CTCCTGTCACTGGTTATCACCCTTTACTGCAACCACAGGAACCGAAGAC |
| | | GTGTTTGCAAACGGGGCAGAAAGAAACTCCTGTATATATTCAAACAACC |
| | | ATTTATGAGACCAGTACAAACTACTCAAGAGGAAGATGGCTGTAGCTGC |
| | | CGATTTCCAGAAGAAGAAGAAGGAGGATGTGAACTACGCGTTAAGTTCA |
| | | GCAGGAGCGCAGACGCCCCCGCGTACCAGCAGGGCCAGAACCAGCTC |
| | | TATAACGAGCTCAATCTAGGACGAAGAGAGGAGTACGATGTTTTGGACA |
| | | AGCGCCGTGGCCGGGACCCTGAGATGGGGGGAAAGCCGCAGAGAAGG |
| | | AAGAACCCTCAGGAAGGCCTGTACAATGAACTGCAGAAAGATAAGATGG |
| | | CGGAGGCCTACAGTGAGATTGGGATGAAAGGCGAGCGCCGGAGGGGC |
| | | AAGGGGCACGATGGCCTTTACCAGGGTCTCAGTACAGCCACCAAGGAC |
| | | ACCTACGACGCCCTTCACATGCAGGCCCTGCCCCCTCGC |
| 74 | CAR comprising Antibody 2 | ATGGCCCTTCCCGTTACCGCCTTACTCTTACCTCTTGCTTTATTACTGCA |
| | nucleic acid | CGCGGCACGGCCTGAAGTCCAACTCGTCGAAAGCGGGGGAGGCTTGG |
| | Single-2 | TACAGCCTGGGGGGTCCCTGAGACTCTCCTGTGCAGCCTCTGGATTCA |
| | | CCTTTAGCAACTATGACATGAGCTGGGTCCGCCAGGCTCCAGGGAAGC |
| | | AGCTTGAGTGGGTCTCAGCTATTACCGGTAGTGGTGGTAGTACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGCTGCAGGCGTAGATGGCGACTATTGGGGCCAGGG |
| | | AACCCTCGTGACAGTTAGCAGCACTACCACACCAGCTCCCAGGCCACC |
| | | CACGCCTGCACCGACAATCGCATCTCAACCACTCTCACTCCGCCCCGAA |
| | | GCGTGCAGACCCGCAGCAGGTGGAGCCGTACATACTAGGGGTCTTGAC |
| | | TTCGCCTGTGATATATATATTTGGGCGCCCTTGGCCGGGACTTGTGGGG |
| | | TCCTTCTCCTGTCACTGGTTATCACCCTTTACTGCAACCACAGGAACCG |
| | | AAGACGTGTTTGCAAACGGGGCAGAAAGAAACTCCTGTATATATTCAAA |
| | | CAACCATTTATGAGACCAGTACAAACTACTCAAGAGGAAGATGGCTGTA |
| | | GCTGCCGATTTCCAGAAGAAGAAGAAGGAGGATGTGAACTACGCGTTAA |
| | | GTTCAGCAGGAGCGCAGACGCCCCCGCGTACCAGCAGGGCCAGAACC |
| | | AGCTCTATAACGAGCTCAATCTAGGACGAAGAGAGGAGTACGATGTTTT |
| | | GGACAAGCGCCGTGGCCGGGACCCTGAGATGGGGGGAAAGCCGCAGA |
| | | GAAGGAAGAACCCTCAGGAAGGCCTGTACAATGAACTGCAGAAAGATAA |
| | | GATGGCGGAGGCCTACAGTGAGATTGGGATGAAAGGCGAGCGCCGGA |
| | | GGGGCAAGGGGCACGATGGCCTTTACCAGGGTCTCAGTACAGCCACCA |
| | | AGGACACCTACGACGCCCTTCACATGCAGGCCCTGCCCCCTCGC |
| 75 | CAR comprising Antibody 3 | ATGGCCCTTCCCGTTACCGCCTTACTCTTACCTCTTGCTTTATTACTGCA |
| | nucleic acid | CGCGGCACGGCCTGAGGTGCAGCTGGTGGAGTCTGGGGGAGGCTTGG |
| | Single-3 | TACAGCCTGGGGGGTCCCTGAGACTCTCCTGTGCAGCCTCTGGATTCA |
| | | CCTTTAGCGCGTATGATATGAGCTGGGTCCGCCAGGCTCCAGGGAAGC |
| | | AGCTTGAGTGGGTCTCAGCTATTAGCGGTAGTGGTGGTAGCACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGCCTACGATGGTGATTCTACAGGGTCTCTGGATGAG |
| | | ATGGCGCACTGGGGCCAGGGAACCCTGGTCACCGTTTCATCTACTACC |
| | | ACACCAGCTCCCAGGCCACCCACGCCTGCACCGACAATCGCATCTCAA |
| | | CCACTCTCACTCCGCCCCGAAGCGTGCAGACCCGCAGCAGGTGGAGC |
| | | CGTACATACTAGGGGTCTTGACTTCGCCTGTGATATATATATTTGGGCG |
| | | CCCTTGGCCGGGACTTGTGGGGTCCTTCTCCTGTCACTGGTTATCACCC |
| | | TTTACTGCAACCACAGGAACCGAAGACGTGTTTGCAAACGGGGCAGAAA |
| | | GAAACTCCTGTATATATTCAAACAACCATTTATGAGACCAGTACAAACTA |
| | | CTCAAGAGGAAGATGGCTGTAGCTGCCGATTTCCAGAAGAAGAAGAAG |
| | | GAGGATGTGAACTACGCGTTAAGTTCAGCAGGAGCGCAGACGCCCCCG |
| | | CGTACCAGCAGGGCCAGAACCAGCTCTATAACGAGCTCAATCTAGGAC |
| | | GAAGAGAGGAGTACGATGTTTTGGACAAGCGCCGTGGCCGGGACCCTG |
| | | AGATGGGGGGAAAGCCGCAGAGAAGGAAGAACCCTCAGGAAGGCCTG |
| | | TACAATGAACTGCAGAAAGATAAGATGGCGGAGGCCTACAGTGAGATTG |
| | | GGATGAAAGGCGAGCGCCGGAGGGGCAAGGGGCACGATGGCCTTTAC |
| | | CAGGGTCTCAGTACAGCCACCAAGGACACCTACGACGCCCTTCACATG |
| | | CAGGCCCTGCCCCCTCGC |
| 76 | CAR comprising Antibody 4 | ATGGCCCTTCCCGTTACCGCCTTACTCTTACCTCTTGCTTTATTACTGCA |
| | nucleic acid | CGCGGCACGGCCTGAGGTGCAGCTGGTGGAGTCTGGGGGAGGCTTGG |
| | Single-4A | TACAGCCTGGGGGGTCCCTGAGACTCTCCTGTGCAGCCTCTGGATTCA |
| | | CCTTTAGCACGTATGCCATGAGCTGGGTCCGCCAGGCTCCAGGGAAGC |
| | | AGCGTGAGTGGGTCTCAGCTATTGAAGGTAGTGGTGGTTCTACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGCCGTAGAGACAGGATGGGCGGATTTTGACTATTGG |
| | | GGCCAGGGAACCCTGGTCACCGTTTCATCTACTACCACACCAGCTCCCA |
| | | GGCCACCCACGCCTGCACCGACAATCGCATCTCAACCACTCTCACTCC |
| | | GCCCCGAAGCGTGCAGACCCGCAGCAGGTGGAGCCGTACATACTAGG |
| | | GGTCTTGACTTCGCCTGTGATATATATATTTGGGCGCCCTTGGCCGGGA |
| | | CTTGTGGGGTCCTTCTCCTGTCACTGGTTATCACCCTTTACTGCAACCAC |
| | | AGGAACCGAAGACGTGTTTGCAAACGGGGCAGAAAGAAACTCCTGTATA |
| | | TATTCAAACAACCATTTATGAGACCAGTACAAACTACTCAAGAGGAAGAT |
| | | GGCTGTAGCTGCCGATTTCCAGAAGAAGAAGAAGGAGGATGTGAACTA |
| | | CGCGTTAAGTTCAGCAGGAGCGCAGACGCCCCCGCGTACCAGCAGGG |
| | | CCAGAACCAGCTCTATAACGAGCTCAATCTAGGACGAAGAGAGGAGTAC |
| | | GATGTTTTGGACAAGCGCCGTGGCCGGGACCCTGAGATGGGGGGAAA |
| | | GCCGCAGAGAAGGAAGAACCCTCAGGAAGGCCTGTACAATGAACTGCA |
| | | GAAAGATAAGATGGCGGAGGCCTACAGTGAGATTGGGATGAAAGGCGA |
| | | GCGCCGGAGGGGCAAGGGGCACGATGGCCTTTACCAGGGTCTCAGTA |
| | | CAGCCACCAAGGACACCTACGACGCCCTTCACATGCAGGCCCTGCCCC |
| | | CTCGC |
| 77 | CAR comprising Antibody 5 | ATGGCCCTTCCCGTTACCGCCTTACTCTTACCTCTTGCTTTATTACTGCA |
| | nucleic acid | CGCGGCACGGCCTGAAGTCCAACTCGTCGAAAGCGGGGGAGGCTTGG |
| | Single-5A | TACAGCCTGGGGGGTCCCTGAGACTCTCCTGTGCAGCCTCTGGATTCA |
| | | CCTTTAGCTACTATGCCATGAGCTGGGTCCGCCAGGCTCCAGGGAAGC |
| | | AGCGTGAGTGGGTCTCAGCTATTGAAGGTAGTGGTGGTCGGACATACT |
| | | ACGCAGACTCCGTGAAGGGCCGGTTCACCATCTCCAGAGACAATTCCA |
| | | AGAACACGCTGTATCTGCAAATGAACAGCCTGAGAGCCGAGGACACGG |
| | | CCGTATATTACTGTGTTAAAGAGACAGGATGGGCGGATTTTGACTATTG |
| | | GGGCCAGGGAACCCTCGTGACAGTTAGCAGCACTACCACACCAGCTCC |
| | | CAGGCCACCCACGCCTGCACCGACAATCGCATCTCAACCACTCTCACTC |
| | | CGCCCCGAAGCGTGCAGACCCGCAGCAGGTGGAGCCGTACATACTAG |
| | | GGGTCTTGACTTCGCCTGTGATATATATATTTGGGCGCCCTTGGCCGGG |
| | | ACTTGTGGGGTCCTTCTCCTGTCACTGGTTATCACCCTTTACTGCAACCA |
| | | CAGGAACCGAAGACGTGTTTGCAAACGGGGCAGAAAGAAACTCCTGTA |
| | | TATATTCAAACAACCATTTATGAGACCAGTACAAACTACTCAAGAGGAAG |
| | | ATGGCTGTAGCTGCCGATTTCCAGAAGAAGAAGAAGGAGGATGTGAACT |
| | | ACGCGTTAAGTTCAGCAGGAGCGCAGACGCCCCCGCGTACCAGCAGG |
| | | GCCAGAACCAGCTCTATAACGAGCTCAATCTAGGACGAAGAGAGGAGT |
| | | ACGATGTTTTGGACAAGCGCCGTGGCCGGGACCCTGAGATGGGGGGA |
| | | AAGCCGCAGAGAAGGAAGAACCCTCAGGAAGGCCTGTACAATGAACTG |
| | | CAGAAAGATAAGATGGCGGAGGCCTACAGTGAGATTGGGATGAAAGGC |
| | | GAGCGCCGGAGGGGCAAGGGGCACGATGGCCTTTACCAGGGTCTCAG |
| | | TACAGCCACCAAGGACACCTACGACGCCCTTCACATGCAGGCCCTGCC |
| | | CCCTCGC |
| 78 | CAR comprising Antibody 4 | ATGGCCCTTCCCGTGACGGCTCTTCTTTTGCCACTGGCACTTCTTTTGC |
| | nucleic acid | ACGCAGCGCGACCTGAGGTGCAGCTGGTGGAGTCTGGGGGAGGCTTG |
| | (TNFRSF4 | GTACAGCCTGGGGGGTCCCTGAGACTCTCCTGTGCAGCCTCTGGATTC |
| | hinge/transmembrane domain) | ACCTTTAGCACGTATGCCATGAGCTGGGTCCGCCAGGCTCCAGGGAAG |
| | Single-4B | CAGCGTGAGTGGGTCTCAGCTATTGAAGGTAGTGGTGGTTCTACATACT |
| | | ACGCAGACTCCGTGAAGGGCCGGTTCACCATCTCCAGAGACAATTCCA |
| | | AGAACACGCTGTATCTGCAAATGAACAGCCTGAGAGCCGAGGACACGG |
| | | CCGTATATTACTGTGCCGTAGAGACAGGATGGGGGGATTTTGACTATTG |
| | | GGGCCAGGGAACCCTGGTCACCGTTTCATCTACGCAGCCCCAGGAGAC |
| | | CCAGGGCCCCCCGGCCAGGCCCATCACTGTCCAGCCCACTGAAGCCT |
| | | GGCCCAGAACCTCACAGGGACCCTCCACCCGGCCCGTGGAGGTCCCC |
| | | GGGGGCCGTGCGGTTGCCGCCATCCTGGGCCTGGGCCTGGTGCTGGG |
| | | GCTGCTGGGCCCCCTGGCCATCCTGCTGGCCCTGTACCTGCTGCGGAG |
| | | GGACCAGAGGAAACGGGGCAGAAAGAAACTCCTGTATATATTCAAACAA |
| | | CCATTTATGAGACCAGTACAAACTACTCAAGAGGAAGATGGCTGTAGCT |
| | | GCCGATTTCCAGAAGAAGAAGAAGGAGGATGTGAACTACGCGTTAAGTT |
| | | CAGCAGGAGCGCAGACGCCCCCGCGTACCAGCAGGGCCAGAACCAGC |
| | | TCTATAACGAGCTCAATCTAGGACGAAGAGAGGAGTACGATGTTTTGGA |
| | | CAAGCGCCGTGGCCGGGACCCTGAGATGGGGGGAAAGCCGCAGAGAA |
| | | GGAAGAACCCTCAGGAAGGCCTGTACAATGAACTGCAGAAAGATAAGAT |
| | | GGCGGAGGCCTACAGTGAGATTGGGATGAAAGGCGAGCGCCGGAGGG |
| | | GCAAGGGGCACGATGGCCTTTACCAGGGTCTCAGTACAGCCACCAAGG |
| | | ACACCTACGACGCCCTTCACATGCAGGCCCTGCCCCCTCGC |
| 79 | CAR comprising Antibody 5 | ATGGCCCTTCCCGTGACGGCTCTTCTTTTGCCACTGGCACTTCTTTTGC |
| | nucleic acid | ACGCAGCGCGACCTGAAGTCCAACTCGTCGAAAGCGGGGGAGGCTTG |
| | (TNFRSF4 | GTACAGCCTGGGGGGTCCCTGAGACTCTCCTGTGCAGCCTCTGGATTC |
| | hinge/transmembrane domain) | ACCTTTAGCTACTATGCCATGAGCTGGGTCCGCCAGGCTCCAGGGAAG |
| | Single-5B | CAGCGTGAGTGGGTCTCAGCTATTGAAGGTAGTGGTGGTCGGACATAC |
| | | TACGCAGACTCCGTGAAGGGCCGGTTCACCATCTCCAGAGACAATTCCA |
| | | AGAACACGCTGTATCTGCAAATGAACAGCCTGAGAGCCGAGGACACGG |
| | | CCGTATATTACTGTGTTAAAGAGACAGGATGGGCGGATTTTGACTATTG |
| | | GGGCCAGGGAACCCTCGTGACAGTTAGCAGCACGCAGCCCCAGGAGA |
| | | CCCAGGGCCCCCCGGCCAGGCCCATCACTGTCCAGCCCACTGAAGCCT |
| | | GGCCCAGAACCTCACAGGGACCCTCCACCCGGCCCGTGGAGGTCCCC |
| | | GGGGGCCGTGCGGTTGCCGCCATCCTGGGCCTGGGCCTGGTGCTGGG |
| | | GCTGCTGGGCCCCCTGGCCATCCTGCTGGCCCTGTACCTGCTGCGGAG |
| | | GGACCAGAGGAAACGGGGCAGAAAGAAACTCCTGTATATATTCAAACAA |
| | | CCATTTATGAGACCAGTACAAACTACTCAAGAGGAAGATGGCTGTAGCT |
| | | GCCGATTTCCAGAAGAAGAAGAAGGAGGATGTGAACTACGCGTTAAGTT |
| | | CAGCAGGAGCGCAGACGCCCCCGCGTACCAGCAGGGCCAGAACCAGC |
| | | TCTATAACGAGCTCAATCTAGGACGAAGAGAGGAGTACGATGTTTTGGA |
| | | CAAGCGCCGTGGCCGGGACCCTGAGATGGGGGGAAAGCCGCAGAGAA |
| | | GGAAGAACCCTCAGGAAGGCCTGTACAATGAACTGCAGAAAGATAAGAT |
| | | GGCGGAGGCCTACAGTGAGATTGGGATGAAAGGCGAGCGCCGGAGGG |
| | | GCAAGGGGCACGATGGCCTTTACCAGGGTCTCAGTACAGCCACCAAGG |
| | | ACACCTACGACGCCCTTCACATGCAGGCCCTGCCCCCTCGC |
| 80 | Tandem CAR comprising | ATGGCCCTTCCCGTTACCGCCTTACTCTTACCTCTTGCTTTATTACTGCA |
| | Antibodies 1 and 4 nucleic acid | CGCGGCACGGCCTGAAGTCCAACTCGTCGAAAGCGGGGGAGGCTTGG |
| | Tandem-1 | TACAGCCTGGGGGGTCCCTGAGACTCTCCTGTGCAGCCTCTGGATTCA |
| | | CCTTTAGCACATATGATATGAGCTGGGTCCGCCAGGCTCCAGGGAAGC |
| | | AGCGTGAGTGGGTCTCAGCTATTTCCGGTAGTGGTGGTACAACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGTGCGCGGCGACGACCATTGGGGCCAGGGAACCCT |
| | | CGTGACAGTTAGCAGCGGCGGAGGGGGATCTGGAGGTGGAGGATCAG |
| | | GGGGAGGTGGTTCCGAGGTGCAGCTGGTGGAGTCTGGGGGAGGCTTG |
| | | GTACAGCCTGGGGGGTCCCTGAGACTCTCCTGTGCAGCCTCTGGATTC |
| | | ACCTTTAGCACGTATGCCATGAGCTGGGTCCGCCAGGCTCCAGGGAAG |
| | | CAGCGTGAGTGGGTCTCAGCTATTGAAGGTAGTGGTGGTTCTACATACT |
| | | ACGCAGACTCCGTGAAGGGCCGGTTCACCATCTCCAGAGACAATTCCA |
| | | AGAACACGCTGTATCTGCAAATGAACAGCCTGAGAGCCGAGGACACGG |
| | | CCGTATATTACTGTGCCGTAGAGACAGGATGGGCGGATTTTGACTATTG |
| | | GGGCCAGGGAACCCTGGTCACCGTTTCATCTACTACCACACCAGCTCC |
| | | CAGGCCACCCACGCCTGCACCGACAATCGCATCTCAACCACTCTCACTC |
| | | CGCCCCGAAGCGTGCAGACCCGCAGCAGGTGGAGCCGTACATACTAG |
| | | GGGTCTTGACTTCGCCTGTGATATATATATTTGGGCGCCCTTGGCCGGG |
| | | ACTTGTGGGGTCCTTCTCCTGTCACTGGTTATCACCCTTTACTGCAACCA |
| | | CAGGAACCGAAGACGTGTTTGCAAACGGGGCAGAAAGAAACTCCTGTA |
| | | TATATTCAAACAACCATTTATGAGACCAGTACAAACTACTCAAGAGGAAG |
| | | ATGGCTGTAGCTGCCGATTTCCAGAAGAAGAAGAAGGAGGATGTGAACT |
| | | ACGCGTTAAGTTCAGCAGGAGCGCAGACGCCCCCGCGTACCAGCAGG |
| | | GCCAGAACCAGCTCTATAACGAGCTCAATCTAGGACGAAGAGAGGAGT |
| | | ACGATGTTTTGGACAAGCGCCGTGGCCGGGACCCTGAGATGGGGGGA |
| | | AAGCCGCAGAGAAGGAAGAACCCTCAGGAAGGCCTGTACAATGAACTG |
| | | CAGAAAGATAAGATGGCGGAGGCCTACAGTGAGATTGGGATGAAAGGC |
| | | GAGCGCCGGAGGGGCAAGGGGCACGATGGCCTTTACCAGGGTCTCAG |
| | | TACAGCCACCAAGGACACCTACGACGCCCTTCACATGCAGGCCCTGCC |
| | | CCCTCGC |
| 81 | Tandem CAR comprising | ATGGCCCTTCCCGTTACCGCCTTACTCTTACCTCTTGCTTTATTACTGCA |
| | Antibodies 2 and 4 nucleic acid | CGCGGCACGGCCTGAAGTCCAACTCGTCGAAAGCGGGGGAGGCTTGG |
| | Tandem-2 | TACAGCCTGGGGGGTCCCTGAGACTCTCCTGTGCAGCCTCTGGATTCA |
| | | CCTTTAGCAACTATGACATGAGCTGGGTCCGCCAGGCTCCAGGGAAGC |
| | | AGCTTGAGTGGGTCTCAGCTATTACCGGTAGTGGTGGTAGTACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGCTGCAGGCGTAGATGGCGACTATTGGGGCCAGGG |
| | | AACCCTCGTGACAGTTAGCAGCGGCGGAGGGGGATCTGGAGGTGGAG |
| | | GATCAGGGGGAGGTGGTTCCGAGGTGCAGCTGGTGGAGTCTGGGGGA |
| | | GGCTTGGTACAGCCTGGGGGGTCCCTGAGACTCTCCTGTGCAGCCTCT |
| | | GGATTCACCTTTAGCACGTATGCCATGAGCTGGGTCCGCCAGGCTCCA |
| | | GGGAAGCAGCGTGAGTGGGTCTCAGCTATTGAAGGTAGTGGTGGTTCT |
| | | ACATACTACGCAGACTCCGTGAAGGGCCGGTTCACCATCTCCAGAGAC |
| | | AATTCCAAGAACACGCTGTATCTGCAAATGAACAGCCTGAGAGCCGAGG |
| | | ACACGGCCGTATATTACTGTGCCGTAGAGACAGGATGGGCGGATTTTGA |
| | | CTATTGGGGCCAGGGAACCCTGGTCACCGTTTCATCTACTACCACACCA |
| | | GCTCCCAGGCCACCCACGCCTGCACCGACAATCGCATCTCAACCACTC |
| | | TCACTCCGCCCCGAAGCGTGCAGACCCGCAGCAGGTGGAGCCGTACAT |
| | | ACTAGGGGTCTTGACTTCGCCTGTGATATATATATTTGGGCGCCCTTGG |
| | | CCGGGACTTGTGGGGTCCTTCTCCTGTCACTGGTTATCACCCTTTACTG |
| | | CAACCACAGGAACCGAAGACGTGTTTGCAAACGGGGCAGAAAGAAACT |
| | | CCTGTATATATTCAAACAACCATTTATGAGACCAGTACAAACTACTCAAG |
| | | AGGAAGATGGCTGTAGCTGCCGATTTCCAGAAGAAGAAGAAGGAGGAT |
| | | GTGAACTACGCGTTAAGTTCAGCAGGAGCGCAGACGCCCCCGCGTACC |
| | | AGCAGGGCCAGAACCAGCTCTATAACGAGCTCAATCTAGGACGAAGAG |
| | | AGGAGTACGATGTTTTGGACAAGCGCCGTGGCCGGGACCCTGAGATGG |
| | | GGGGAAAGCCGCAGAGAAGGAAGAACCCTCAGGAAGGCCTGTACAATG |
| | | AACTGCAGAAAGATAAGATGGCGGAGGCCTACAGTGAGATTGGGATGA |
| | | AAGGCGAGCGCCGGAGGGGCAAGGGGCACGATGGCCTTTACCAGGGT |
| | | CTCAGTACAGCCACCAAGGACACCTACGACGCCCTTCACATGCAGGCC |
| | | CTGCCCCCTCGC |
| 82 | Tandem CAR comprising | ATGGCCCTTCCCGTTACCGCCTTACTCTTACCTCTTGCTTTATTACTGCA |
| | Antibodies 5 and 3 nucleic acid | CGCGGCACGGCCTGAAGTCCAACTCGTCGAAAGCGGGGGAGGCTTGG |
| | Tandem-3 | TACAGCCTGGGGGGTCCCTGAGACTCTCCTGTGCAGCCTCTGGATTCA |
| | | CCTTTAGCTACTATGCCATGAGCTGGGTCCGCCAGGCTCCAGGGAAGC |
| | | AGCGTGAGTGGGTCTCAGCTATTGAAGGTAGTGGTGGTCGGACATACT |
| | | ACGCAGACTCCGTGAAGGGCCGGTTCACCATCTCCAGAGACAATTCCA |
| | | AGAACACGCTGTATCTGCAAATGAACAGCCTGAGAGCCGAGGACACGG |
| | | CCGTATATTACTGTGTTAAAGAGACAGGATGGGCGGATTTTGACTATTG |
| | | GGGCCAGGGAACCCTCGTGACAGTTAGCAGCGGCGGAGGGGGATCTG |
| | | GAGGTGGAGGATCAGGGGGAGGTGGTTCCGAGGTGCAGCTGGTGGAG |
| | | TCTGGGGGAGGCTTGGTACAGCCTGGGGGGTCCCTGAGACTCTCCTGT |
| | | GCAGCCTCTGGATTCACCTTTAGCGCGTATGATATGAGCTGGGTCCGCC |
| | | AGGCTCCAGGGAAGCAGCTTGAGTGGGTCTCAGCTATTAGCGGTAGTG |
| | | GTGGTAGCACATACTACGCAGACTCCGTGAAGGGCCGGTTCACCATCT |
| | | CCAGAGACAATTCCAAGAACACGCTGTATCTGCAAATGAACAGCCTGAG |
| | | AGCCGAGGACACGGCCGTATATTACTGTGCCTACGATGGTGATTCTACA |
| | | GGGTCTCTGGATGAGATGGCGCACTGGGGCCAGGGAACCCTGGTCAC |
| | | CGTTTCATCTACTACCACACCAGCTCCCAGGCCACCCACGCCTGCACC |
| | | GACAATCGCATCTCAACCACTCTCACTCCGCCCCGAAGCGTGCAGACC |
| | | CGCAGCAGGTGGAGCCGTACATACTAGGGGTCTTGACTTCGCCTGTGA |
| | | TATATATATTTGGGCGCCCTTGGCCGGGACTTGTGGGGTCCTTCTCCTG |
| | | TCACTGGTTATCACCCTTTACTGCAACCACAGGAACCGAAGACGTGTTT |
| | | GCAAACGGGGCAGAAAGAAACTCCTGTATATATTCAAACAACCATTTATG |
| | | AGACCAGTACAAACTACTCAAGAGGAAGATGGCTGTAGCTGCCGATTTC |
| | | CAGAAGAAGAAGAAGGAGGATGTGAACTACGCGTTAAGTTCAGCAGGA |
| | | GCGCAGACGCCCCCGCGTACCAGCAGGGCCAGAACCAGCTCTATAACG |
| | | AGCTCAATCTAGGACGAAGAGAGGAGTACGATGTTTTGGACAAGCGCC |
| | | GTGGCCGGGACCCTGAGATGGGGGGAAAGCCGCAGAGAAGGAAGAAC |
| | | CCTCAGGAAGGCCTGTACAATGAACTGCAGAAAGATAAGATGGCGGAG |
| | | GCCTACAGTGAGATTGGGATGAAAGGCGAGCGCCGGAGGGGCAAGGG |
| | | GCACGATGGCCTTTACCAGGGTCTCAGTACAGCCACCAAGGACACCTA |
| | | CGACGCCCTTCACATGCAGGCCCTGCCCCCTCGC |
| 83 | Parallel CAR comprising | ATGGCCCTTCCCGTTACCGCCTTACTCTTACCTCTTGCTTTATTACTGCA |
| | Antibodies 1 and 4 nucleic acid | CGCGGCACGGCCTGAAGTCCAACTCGTCGAAAGCGGGGGAGGCTTGG |
| | Parallel-1 | TACAGCCTGGGGGGTCCCTGAGACTCTCCTGTGCAGCCTCTGGATTCA |
| | | CCTTTAGCACATATGATATGAGCTGGGTCCGCCAGGCTCCAGGGAAGC |
| | | AGCGTGAGTGGGTCTCAGCTATTTCCGGTAGTGGTGGTACAACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGTGCGCGGCGACGACCATTGGGGCCAGGGAACCCT |
| | | CGTGACAGTTAGCAGCACTACGACTCCTGCACCGCGACCGCCCACACC |
| | | AGCTCCTACCATAGCTTCCCAACCTCTCTCTCTTCGCCCAGAAGCGTGT |
| | | CGGCCTGCCGCGGGTGGCGCAGTACACACGAGGGGCCTGGACTTTGC |
| | | ATGTGATATCTACATCTGGGCTCCACTCGCAGGAACCTGTGGCGTATTG |
| | | CTTCTTTCCTTGGTGATTACTCTCTATTGCAATCACAGAAATAGGAGGCG |
| | | AGTGTGTAAACGCGGGCGCAAGAAGTTGTTGTACATATTCAAGCAACCC |
| | | TTTATGAGACCAGTGCAAACAACACAAGAAGAGGATGGTTGTTCATGCA |
| | | GATTCCCTGAGGAGGAAGAGGGCGGCTGCGAACTTAGGGTCAAGTTTT |
| | | CAAGGTCCGCTGACGCACCGGCATACCAGCAAGGCCAAAATCAGCTTT |
| | | ATAATGAACTGAATCTCGGGCGCCGAGAAGAATATGATGTTCTTGACAA |
| | | GAGGCGCGGGAGGGACCCCGAAATGGGGGGCAAACCCCAGCGGAGG |
| | | AAGAATCCCCAGGAAGGCCTTTACAATGAATTGCAGAAAGACAAAATGG |
| | | CTGAAGCATACAGTGAAATAGGAATGAAGGGGGAGAGGCGCCGCGGG |
| | | AAGGGACATGACGGATTGTATCAGGGATTGAGTACTGCGACGAAAGAC |
| | | ACGTACGATGCTCTTCACATGCAAGCGCTTCCACCGCGGGAGGGCAGA |
| | | GGAAGTCTTCTAACATGCGGTGACGTGGAGGAGAATCCCGGCCCTATG |
| | | GCCCTTCCCGTGACGGCTCTTCTTTTGCCACTGGCACTTCTTTTGCACG |
| | | CAGCGCGACCTGAGGTGCAGCTGGTGGAGTCTGGGGGAGGCTTGGTA |
| | | CAGCCTGGGGGGTCCCTGAGACTCTCCTGTGCAGCCTCTGGATTCACC |
| | | TTTAGCACGTATGCCATGAGCTGGGTCCGCCAGGCTCCAGGGAAGCAG |
| | | CGTGAGTGGGTCTCAGCTATTGAAGGTAGTGGTGGTTCTACATACTACG |
| | | CAGACTCCGTGAAGGGCCGGTTCACCATCTCCAGAGACAATTCCAAGAA |
| | | CACGCTGTATCTGCAAATGAACAGCCTGAGAGCCGAGGACACGGCCGT |
| | | ATATTACTGTGCCGTAGAGACAGGATGGGCGGATTTTGACTATTGGGGC |
| | | CAGGGAACCCTGGTCACCGTTTCATCTACGCAGCCCCAGGAGACCCAG |
| | | GGCCCCCCGGCCAGGCCCATCACTGTCCAGCCCACTGAAGCCTGGCC |
| | | CAGAACCTCACAGGGACCCTCCACCCGGCCCGTGGAGGTCCCCGGGG |
| | | GCCGTGCGGTTGCCGCCATCCTGGGCCTGGGCCTGGTGCTGGGGCTG |
| | | CTGGGCCCCCTGGCCATCCTGCTGGCCCTGTACCTGCTGCGGAGGGA |
| | | CCAGAGGAAACGGGGCAGAAAGAAACTCCTGTATATATTCAAACAACCA |
| | | TTTATGAGACCAGTACAAACTACTCAAGAGGAAGATGGCTGTAGCTGCC |
| | | GATTTCCAGAAGAAGAAGAAGGAGGATGTGAACTACGCGTTAAGTTCAG |
| | | CAGGAGCGCAGACGCCCCCGCGTACCAGCAGGGCCAGAACCAGCTCT |
| | | ATAACGAGCTCAATCTAGGACGAAGAGAGGAGTACGATGTTTTGGACAA |
| | | GCGCCGTGGCCGGGACCCTGAGATGGGGGGAAAGCCGCAGAGAAGGA |
| | | AGAACCCTCAGGAAGGCCTGTACAATGAACTGCAGAAAGATAAGATGGC |
| | | GGAGGCCTACAGTGAGATTGGGATGAAAGGCGAGCGCCGGAGGGGCA |
| | | AGGGGCACGATGGCCTTTACCAGGGTCTCAGTACAGCCACCAAGGACA |
| | | CCTACGACGCCCTTCACATGCAGGCCCTGCCCCCTCGC |
| 84 | Parallel CAR comprising | ATGGCCCTTCCCGTTACCGCCTTACTCTTACCTCTTGCTTTATTACTGCA |
| | Antibodies 2 and | CGCGGCACGGCCTGAAGTCCAACTCGTCGAAAGCGGGGGAGGCTTGG |
| | 4 nucleic acid | TACAGCCTGGGGGGTCCCTGAGACTCTCCTGTGCAGCCTCTGGATTCA |
| | Parallel-2 | CCTTTAGCAACTATGACATGAGCTGGGTCCGCCAGGCTCCAGGGAAGC |
| | | AGCTTGAGTGGGTCTCAGCTATTACCGGTAGTGGTGGTAGTACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGCTGCAGGCGTAGATGGCGACTATTGGGGCCAGGG |
| | | AACCCTCGTGACAGTTAGCAGCACTACGACTCCTGCACCGCGACCGCC |
| | | CACACCAGCTCCTACCATAGCTTCCCAACCTCTCTCTCTTCGCCCAGAA |
| | | GCGTGTCGGCCTGCCGCGGGTGGCGCAGTACACACGAGGGGCCTGGA |
| | | CTTTGCATGTGATATCTACATCTGGGCTCCACTCGCAGGAACCTGTGGC |
| | | GTATTGCTTCTTTCCTTGGTGATTACTCTCTATTGCAATCACAGAAATAG |
| | | GAGGCGAGTGTGTAAACGCGGGCGCAAGAAGTTGTTGTACATATTCAA |
| | | GCAACCCTTTATGAGACCAGTGCAAACAACACAAGAAGAGGATGGTTGT |
| | | TCATGCAGATTCCCTGAGGAGGAAGAGGGCGGCTGCGAACTTAGGGTC |
| | | AAGTTTTCAAGGTCCGCTGACGCACCGGCATACCAGCAAGGCCAAAATC |
| | | AGCTTTATAATGAACTGAATCTCGGGCGCCGAGAAGAATATGATGTTCTT |
| | | GACAAGAGGCGCGGGAGGGACCCCGAAATGGGGGGCAAACCCCAGCG |
| | | GAGGAAGAATCCCCAGGAAGGCCTTTACAATGAATTGCAGAAAGACAAA |
| | | ATGGCTGAAGCATACAGTGAAATAGGAATGAAGGGGGAGAGGCGCCGC |
| | | GGGAAGGGACATGACGGATTGTATCAGGGATTGAGTACTGCGACGAAA |
| | | GACACGTACGATGCTCTTCACATGCAAGCGCTTCCACCGCGGGAGGGC |
| | | AGAGGAAGTCTTCTAACATGCGGTGACGTGGAGGAGAATCCCGGCCCT |
| | | ATGGCCCTTCCCGTGACGGCTCTTCTTTTGCCACTGGCACTTCTTTTGC |
| | | ACGCAGCGCGACCTGAGGTGCAGCTGGTGGAGTCTGGGGGAGGCTTG |
| | | GTACAGCCTGGGGGGTCCCTGAGACTCTCCTGTGCAGCCTCTGGATTC |
| | | ACCTTTAGCACGTATGCCATGAGCTGGGTCCGCCAGGCTCCAGGGAAG |
| | | CAGCGTGAGTGGGTCTCAGCTATTGAAGGTAGTGGTGGTTCTACATACT |
| | | ACGCAGACTCCGTGAAGGGCCGGTTCACCATCTCCAGAGACAATTCCA |
| | | AGAACACGCTGTATCTGCAAATGAACAGCCTGAGAGCCGAGGACACGG |
| | | CCGTATATTACTGTGCCGTAGAGACAGGATGGGCGGATTTTGACTATTG |
| | | GGGCCAGGGAACCCTGGTCACCGTTTCATCTACGCAGCCCCAGGAGAC |
| | | CCAGGGCCCCCCGGCCAGGCCCATCACTGTCCAGCCCACTGAAGCCT |
| | | GGCCCAGAACCTCACAGGGACCCTCCACCCGGCCCGTGGAGGTCCCC |
| | | GGGGGCCGTGCGGTTGCCGCCATCCTGGGCCTGGGCCTGGTGCTGGG |
| | | GCTGCTGGGCCCCCTGGCCATCCTGCTGGCCCTGTACCTGCTGCGGAG |
| | | GGACCAGAGGAAACGGGGCAGAAAGAAACTCCTGTATATATTCAAACAA |
| | | CCATTTATGAGACCAGTACAAACTACTCAAGAGGAAGATGGCTGTAGCT |
| | | GCCGATTTCCAGAAGAAGAAGAAGGAGGATGTGAACTACGCGTTAAGTT |
| | | CAGCAGGAGCGCAGACGCCCCCGCGTACCAGCAGGGCCAGAACCAGC |
| | | TCTATAACGAGCTCAATCTAGGACGAAGAGAGGAGTACGATGTTTTGGA |
| | | CAAGCGCCGTGGCCGGGACCCTGAGATGGGGGGAAAGCCGCAGAGAA |
| | | GGAAGAACCCTCAGGAAGGCCTGTACAATGAACTGCAGAAAGATAAGAT |
| | | GGCGGAGGCCTACAGTGAGATTGGGATGAAAGGCGAGCGCCGGAGGG |
| | | GCAAGGGGCACGATGGCCTTTACCAGGGTCTCAGTACAGCCACCAAGG |
| | | ACACCTACGACGCCCTTCACATGCAGGCCCTGCCCCCTCGC |
| 85 | Parallel CAR comprising | ATGGCCCTTCCCGTTACCGCCTTACTCTTACCTCTTGCTTTATTACTGCA |
| | Antibodies 3 and 5 nucleic acid | CGCGGCACGGCCTGAGGTGCAGCTGGTGGAGTCTGGGGGAGGCTTGG |
| | Parellel-3 | TACAGCCTGGGGGGTCCCTGAGACTCTCCTGTGCAGCCTCTGGATTCA |
| | | CCTTTAGCGCGTATGATATGAGCTGGGTCCGCCAGGCTCCAGGGAAGC |
| | | AGCTTGAGTGGGTCTCAGCTATTAGCGGTAGTGGTGGTAGCACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGCCTACGATGGTGATTCTACAGGGTCTCTGGATGAG |
| | | ATGGCGCACTGGGGCCAGGGAACCCTGGTCACCGTTTCATCTACTACG |
| | | ACTCCTGCACCGCGACCGCCCACACCAGCTCCTACCATAGCTTCCCAA |
| | | CCTCTCTCTCTTCGCCCAGAAGCGTGTCGGCCTGCCGCGGGTGGCGCA |
| | | GTACACACGAGGGGCCTGGACTTTGCATGTGATATCTACATCTGGGCTC |
| | | CACTCGCAGGAACCTGTGGCGTATTGCTTCTTTCCTTGGTGATTACTCTC |
| | | TATTGCAATCACAGAAATAGGAGGCGAGTGTGTAAACGCGGGCGCAAG |
| | | AAGTTGTTGTACATATTCAAGCAACCCTTTATGAGACCAGTGCAAACAAC |
| | | ACAAGAAGAGGATGGTTGTTCATGCAGATTCCCTGAGGAGGAAGAGGG |
| | | CGGCTGCGAACTTAGGGTCAAGTTTTCAAGGTCCGCTGACGCACCGGC |
| | | ATACCAGCAAGGCCAAAATCAGCTTTATAATGAACTGAATCTCGGGCGC |
| | | CGAGAAGAATATGATGTTCTTGACAAGAGGCGCGGGAGGGACCCCGAA |
| | | ATGGGGGGCAAACCCCAGCGGAGGAAGAATCCCCAGGAAGGCCTTTAC |
| | | AATGAATTGCAGAAAGACAAAATGGCTGAAGCATACAGTGAAATAGGAA |
| | | TGAAGGGGGAGAGGCGCCGCGGGAAGGGACATGACGGATTGTATCAG |
| | | GGATTGAGTACTGCGACGAAAGACACGTACGATGCTCTTCACATGCAAG |
| | | CGCTTCCACCGCGGGAGGGCAGAGGAAGTCTTCTAACATGCGGTGACG |
| | | TGGAGGAGAATCCCGGCCCTATGGCCCTTCCCGTGACGGCTCTTCTTTT |
| | | GCCACTGGCACTTCTTTTGCACGCAGCGCGACCTGAAGTCCAACTCGTC |
| | | GAAAGCGGGGGAGGCTTGGTACAGCCTGGGGGGTCCCTGAGACTCTC |
| | | CTGTGCAGCCTCTGGATTCACCTTTAGCTACTATGCCATGAGCTGGGTC |
| | | CGCCAGGCTCCAGGGAAGCAGCGTGAGTGGGTCTCAGCTATTGAAGGT |
| | | AGTGGTGGTCGGACATACTACGCAGACTCCGTGAAGGGCCGGTTCACC |
| | | ATCTCCAGAGACAATTCCAAGAACACGCTGTATCTGCAAATGAACAGCC |
| | | TGAGAGCCGAGGACACGGCCGTATATTACTGTGTTAAAGAGACAGGAT |
| | | GGGCGGATTTTGACTATTGGGGCCAGGGAACCCTCGTGACAGTTAGCA |
| | | GCACGCAGCCCCAGGAGACCCAGGGCCCCCCGGCCAGGCCCATCACT |
| | | GTCCAGCCCACTGAAGCCTGGCCCAGAACCTCACAGGGACCCTCCACC |
| | | CGGCCCGTGGAGGTCCCCGGGGGCCGTGCGGTTGCCGCCATCCTGGG |
| | | CCTGGGCCTGGTGCTGGGGCTGCTGGGCCCCCTGGCCATCCTGCTGG |
| | | CCCTGTACCTGCTGCGGAGGGACCAGAGGAAACGGGGCAGAAAGAAA |
| | | CTCCTGTATATATTCAAACAACCATTTATGAGACCAGTACAAACTACTCA |
| | | AGAGGAAGATGGCTGTAGCTGCCGATTTCCAGAAGAAGAAGAAGGAGG |
| | | ATGTGAACTACGCGTTAAGTTCAGCAGGAGCGCAGACGCCCCCGCGTA |
| | | CCAGCAGGGCCAGAACCAGCTCTATAACGAGCTCAATCTAGGACGAAG |
| | | AGAGGAGTACGATGTTTTGGACAAGCGCCGTGGCCGGGACCCTGAGAT |
| | | GGGGGGAAAGCCGCAGAGAAGGAAGAACCCTCAGGAAGGCCTGTACA |
| | | ATGAACTGCAGAAAGATAAGATGGCGGAGGCCTACAGTGAGATTGGGA |
| | | TGAAAGGCGAGCGCCGGAGGGGCAAGGGGCACGATGGCCTTTACCAG |
| | | GGTCTCAGTACAGCCACCAAGGACACCTACGACGCCCTTCACATGCAG |
| | | GCCCTGCCCCCTCGC |
| 86 | CAR comprising Comparative | ATGGCCCTTCCCGTTACCGCCTTACTCTTACCTCTTGCTTTATTACTGCA |
| | Antibody 6 nucleic acid | CGCGGCACGGCCTCAGGTCCAACTGCAAGAAAGCGGACCCGGACTGG |
| | | TGAAGCCTTCTGAGACACTTAGTCTGACGTGCACGGTCAGTGGCGGCT |
| | | CCATCTCCTCCTATTATTGGTCATGGATACGACAACCCCCAGGTAAGGG |
| | | CCTGGAATGGATTGGCTATATCTACTATTCAGGAAGCACGAACTACAAT |
| | | CCCAGCCTGAAGTCCCGAGTGACAATTTCAGTAGATACCAGTAAAAACC |
| | | AGTTCAGTCTTAAACTGTCAAGCGTGACAGCTGCCGACACCGCTGTGTA |
| | | TTACTGCGTCAGCCTGGTGTATTGTGGAGGGGATTGTTATAGCGGGTTC |
| | | GATTATTGGGGACAGGGAACCCTGGTGACTGTATCTTCCGGCGGCGGC |
| | | GGCTCAGGGGGTGGCGGTAGTGGCGGTGGGGGTTCCGATATTCAACT |
| | | GACACAATCCCCCAGCTCACTCAGCGCCAGCGTGGGGGACAGGGTTAG |
| | | CTTTACCTGTCAAGCCTCTCAGGATATAAATAACTTTCTGAACTGGTATC |
| | | AACAGAAGCCTGGGAAGGCGCCCAAACTCCTGATCTATGATGCGTCCA |
| | | ACCTGGAAACTGGCGTGCCTTCACGCTTTAGCGGCTCTGGCAGTGGTA |
| | | CAGACTTCACTTTTACCATCTCTTCACTTCAGCCGGAGGACATCGCCAC |
| | | ATATTACTGTCAACAGTACGGAAACTTGCCCTTTACTTTTGGAGGCGGC |
| | | ACCAAAGTTGAAATCAAAACTACCACACCAGCTCCCAGGCCACCCACGC |
| | | CTGCACCGACAATCGCATCTCAACCACTCTCACTCCGCCCCGAAGCGT |
| | | GCAGACCCGCAGCAGGTGGAGCCGTACATACTAGGGGTCTTGACTTCG |
| | | CCTGTGATATATATATTTGGGCGCCCTTGGCCGGGACTTGTGGGGTCCT |
| | | TCTCCTGTCACTGGTTATCACCCTTTACTGCAACCACAGGAACCGAAGA |
| | | CGTGTTTGCAAACGGGGCAGAAAGAAACTCCTGTATATATTCAAACAAC |
| | | CATTTATGAGACCAGTACAAACTACTCAAGAGGAAGATGGCTGTAGCTG |
| | | CCGATTTCCAGAAGAAGAAGAAGGAGGATGTGAACTACGCGTTAAGTTC |
| | | AGCAGGAGCGCAGACGCCCCCGCGTACCAGCAGGGCCAGAACCAGCT |
| | | CTATAACGAGCTCAATCTAGGACGAAGAGAGGAGTACGATGTTTTGGAC |
| | | AAGCGCCGTGGCCGGGACCCTGAGATGGGGGGAAAGCCGCAGAGAAG |
| | | GAAGAACCCTCAGGAAGGCCTGTACAATGAACTGCAGAAAGATAAGATG |
| | | GCGGAGGCCTACAGTGAGATTGGGATGAAAGGCGAGCGCCGGAGGGG |
| | | CAAGGGGCACGATGGCCTTTACCAGGGTCTCAGTACAGCCACCAAGGA |
| | | CACCTACGACGCCCTTCACATGCAGGCCCTGCCCCCTCGC |
| 87 | CAR comprising Comparative | ATGGCCCTTCCCGTTACCGCCTTACTCTTACCTCTTGCTTTATTACTGCA |
| | Antibody 7 nucleic acid | CGCGGCACGGCCTCAAGTGCAACTGGTACAGAGCGGGGCTGAAGTGA |
| | | AGAAGCCGGGAAGCAGCGTCAAGGTTAGCTGTAAGGCATCTGGCTACA |
| | | CTTTCACTGATTATAACATGCACTGGGTGAGACAAGCACCGGGTCAGGG |
| | | CCTCGAATGGATCGGGTATATTTATCCGTATAACGGGGGCACCGGGTAT |
| | | AATCAGAAGTTTAAGAGCAAGGCCACTATAACGGCCGACGAATCAACCA |
| | | ATACGGCCTATATGGAGCTGTCAAGCCTTAGATCAGAGGACACTGCCGT |
| | | CTACTATTGTGCAAGAGGTAGACCTGCAATGGATTATTGGGGACAGGGT |
| | | ACCTTGGTGACGGTATCTAGTGGCGGAGGGGGATCTGGAGGTGGAGG |
| | | ATCAGGGGGAGGTGGTTCCGATATCCAAATGACTCAATCCCCCTCTTCA |
| | | CTGAGTGCCTCAGTTGGAGACCGCGTTACAATCACATGTAGAGCCTCAG |
| | | AGTCAGTGGATAATTATGGGATTAGCTTCATGAACTGGTTCCAGCAAAA |
| | | GCCAGGTAAGGCGCCTAAACTCCTGATTTATGCGGCATCCAACCAAGG |
| | | CAGTGGGGTTCCGTCACGCTTCTCCGGGTCAGGGTCCGGCACGGACTT |
| | | CACACTGACGATCTCATCATTGCAGCCGGATGACTTCGCAACGTATTAT |
| | | TGCCAGCAGTCAAAAGAAGTTCCCTGGACCTTCGGTCAGGGTACCAAA |
| | | GTCGAAATCAAGACTACCACACCAGCTCCCAGGCCACCCACGCCTGCA |
| | | CCGACAATCGCATCTCAACCACTCTCACTCCGCCCCGAAGCGTGCAGA |
| | | CCCGCAGCAGGTGGAGCCGTACATACTAGGGGTCTTGACTTCGCCTGT |
| | | GATATATATATTTGGGCGCCCTTGGCCGGGACTTGTGGGGTCCTTCTCC |
| | | TGTCACTGGTTATCACCCTTTACTGCAACCACAGGAACCGAAGACGTGT |
| | | TTGCAAACGGGGCAGAAAGAAACTCCTGTATATATTCAAACAACCATTTA |
| | | TGAGACCAGTACAAACTACTCAAGAGGAAGATGGCTGTAGCTGCCGATT |
| | | TCCAGAAGAAGAAGAAGGAGGATGTGAACTACGCGTTAAGTTCAGCAG |
| | | GAGCGCAGACGCCCCCGCGTACCAGCAGGGCCAGAACCAGCTCTATAA |
| | | CGAGCTCAATCTAGGACGAAGAGAGGAGTACGATGTTTTGGACAAGCG |
| | | CCGTGGCCGGGACCCTGAGATGGGGGGAAAGCCGCAGAGAAGGAAGA |
| | | ACCCTCAGGAAGGCCTGTACAATGAACTGCAGAAAGATAAGATGGCGG |
| | | AGGCCTACAGTGAGATTGGGATGAAAGGCGAGCGCCGGAGGGGCAAG |
| | | GGGCACGATGGCCTTTACCAGGGTCTCAGTACAGCCACCAAGGACACC |
| | | TACGACGCCCTTCACATGCAGGCCCTGCCCCCTCGC |
| 88 | GSG-T2A self cleaving peptide | GSGEGRGSLLTCGDVEENPGP |
| 89 | GSG-P2A - GSG-T2A self | GSGATNFSLLKQAGDVEENPGPGSGEGRGSLLTCGDVEENPGP |
| | cleaving peptide | |
| 90 | 2A self cleaving peptide | ARAKRSGSGEGRGSLITCGDVEENPGP |
| 91 | GSG-P2A self cleaving peptide | GSGATNFSLLKQAGDVEENPGP |
| 92 | tCD34 | MPRGWTALCLLSLLPSGFMSLDNNGTATPELPTQGTFSNVSTNVSYQETTT |
| | | PSTLGSTSLHPVSQHGNEATTNITETTVKFTSTSVITSVYGNTNSSVQSQTS |
| | | VISTVFTTPANVSTPETTLKPSLSPGNVSDLSTTSTSLATSPTKPYTSSSPILS |
| | | DIKAEIKCSGIREVKLTQGICLEQNKTSSCAEFKKDRGEGLARVLCGEEQAD |
| | | ADAGAQVCSLLLAQSEVRPQCLLLVLANRTEISSKLQLMKKHQSDLKKLGIL |
| | | DFTEQDVASHQSYSQKTLIALVTSGALLAVLGITGYFLMNRRSWSPTGERL |
| | | ELEP |
| 93 | Tandem CAR comprising | MALPVTALLLPLALLLHAARPEVQLVESGGGLVQPGGSLRLSCAASGFTFS |
| | antibodies 3 and 5 | AYDMSWVRQAPGKQLEWVSAISGSGGSTYYADSVKGRFTISRDNSKNTLY |
| | Tandem-3.2 | LQMNSLRAEDTAVYYCAYDGDSTGSLDEMAHWGQGTLVTVSSGGGGSG |
| | | GGGSGGGGSEVQLVESGGGLVQPGGSLRLSCAASGFTFSYYAMSWVRQ |
| | | APGKQREWVSAIEGSGGRTYYADSVKGRFTISRDNSKNTLYLQMNSLRAE |
| | | DTAVYYCVKETGWADFDYWGQGTLVTVSSTTTPAPRPPTPAPTIASQPLSL |
| | | RPEACRPAAGGAVHTRGLDFACDIYIWAPLAGTCGVLLLSLVITLYCNHRNR |
| | | RRVCKRGRKKLLYIFKQPFMRPVQTTQEEDGCSCRFPEEEEGGCELRVKF |
| | | SRSADAPAYQQGQNQLYNELNLGRREEYDVLDKRRGRDPEMGGKPQRR |
| | | KNPQEGLYNELQKDKMAEAYSEIGMKGERRRGKGHDGLYQGLSTATKDTY |
| | | DALHMQALPPR |
| 94 | Tandem CAR comprising | MALPVTALLLPLALLLHAARPEVQLVESGGGLVQPGGSLRLSCAASGFTFS |
| | antibodies 5 and 3 | YYAMSWVRQAPGKQREWVSAIEGSGGRTYYADSVKGRFTISRDNSKNTLY |
| | with linker 3 | LQMNSLRAEDTAVYYCVKETGWADFDYWGQGTLVTVSSGGGGSEVQLVE |
| | Tandem-3.3 | SGGGLVQPGGSLRLSCAASGFTFSAYDMSWVRQAPGKQLEWVSAISGSG |
| | | GSTYYADSVKGRFTISRDNSKNTLYLQMNSLRAEDTAVYYCAYDGDSTGSL |
| | | DEMAHWGQGTLVTVSSTTTPAPRPPTPAPTIASQPLSLRPEACRPAAGGA |
| | | VHTRGLDFACDIYIWAPLAGTCGVLLLSLVITLYCNHRNRRRVCKRGRKKLL |
| | | YIFKQPFMRPVQTTQEEDGCSCRFPEEEEGGCELRVKFSRSADAPAYQQG |
| | | QNQLYNELNLGRREEYDVLDKRRGRDPEMGGKPQRRKNPQEGLYNELQK |
| | | DKMAEAYSEIGMKGERRRGKGHDGLYQGLSTATKDTYDALHMQALPPR |
| 95 | Parallel CAR comprising | MALPVTALLLPLALLLHAARPEVQLVESGGGLVQPGGSLRLSCAASGFTFS |
| | antibodies 3 and 5 with GSG- | AYDMSWVRQAPGKQLEWVSAISGSGGSTYYADSVKGRFTISRDNSKNTLY |
| | T2A self-cleaving peptide | LQMNSLRAEDTAVYYCAYDGDSTGSLDEMAHWGQGTLVTVSSTTTPAPRP |
| | Parallel-3.2 | PTPAPTIASQPLSLRPEACRPAAGGAVHTRGLDFACDIYIWAPLAGTCGVLL |
| | | LSLVITLYCNHRNRRRVCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPQRRKNPQEGLYNELQKDKMAEAYSEIGMKGERRRGKGHD |
| | | GLYQGLSTATKDTYDALHMQALPPRGSGEGRGSLLTCGDVEENPGPMALP |
| | | VTALLLPLALLLHAARPEVQLVESGGGLVQPGGSLRLSCAASGFTFSYYAM |
| | | SWVRQAPGKQREWVSAIEGSGGRTYYADSVKGRFTISRDNSKNTLYLQMN |
| | | SLRAEDTAVYYCVKETGWADFDYWGQGTLVTVSSTQPQETQGPPARPITV |
| | | QPTEAWPRTSQGPSTRPVEVPGGRAVAAILGLGLVLGLLGPLAILLALYLLR |
| | | RDQRKRGRKKLLYIFKQPFMRPVQTTQEEDGCSCRFPEEEEGGCELRVKF |
| | | SRSADAPAYQQGQNQLYNELNLGRREEYDVLDKRRGRDPEMGGKPQRR |
| | | KNPQEGLYNELQKDKMAEAYSEIGMKGERRRGKGHDGLYQGLSTATKDTY |
| | | DALHMQALPPR |
| 96 | Parallel CAR comprising | MALPVTALLLPLALLLHAARPEVQLVESGGGLVQPGGSLRLSCAASGFTFS |
| | antibodies 3 and 5 with GSG- | AYDMSWVRQAPGKQLEWVSAISGSGGSTYYADSVKGRFTISRDNSKNTLY |
| | P2A - GSG-T2A self-cleaving | LQMNSLRAEDTAVYYCAYDGDSTGSLDEMAHWGQGTLVTVSSTTTPAPRP |
| | peptide | PTPAPTIASQPLSLRPEACRPAAGGAVHTRGLDFACDIYIWAPLAGTCGVLL |
| | Parallel-3.3 | LSLVITLYCNHRNRRRVCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPQRRKNPQEGLYNELQKDKMAEAYSEIGMKGERRRGKGHD |
| | | GLYQGLSTATKDTYDALHMQALPPRGSGATNFSLLKQAGDVEENPGPGSG |
| | | EGRGSLLTCGDVEENPGPMALPVTALLLPLALLLHAARPEVQLVESGGGLV |
| | | QPGGSLRLSCAASGFTFSYYAMSWVRQAPGKQREWVSAIEGSGGRTYYA |
| | | DSVKGRFTISRDNSKNTLYLQMNSLRAEDTAVYYCVKETGWADFDYWGQ |
| | | GTLVTVSSTQPQETQGPPARPITVQPTEAWPRTSQGPSTRPVEVPGGRAV |
| | | AAILGLGLVLGLLGPLAILLALYLLRRDQRKRGRKKLLYIFKQPFMRPVQTTQ |
| | | EEDGCSCRFPEEEEGGCELRVKFSRSADAPAYQQGQNQLYNELNLGRRE |
| | | EYDVLDKRRGRDPEMGGKPQRRKNPQEGLYNELQKDKMAEAYSEIGMKG |
| | | ERRRGKGHDGLYQGLSTATKDTYDALHMQALPPR |
| 97 | Tandem CAR comprising | ATGGCCCTTCCCGTTACCGCCTTACTCTTACCTCTTGCTTTATTACTGCA |
| | antibodies 5 and 3 nucleic acid | CGCGGCACGGCCTGAAGTCCAGTTGGTCGAATCCGGCGGTGGCCTCG |
| | Tandem-3.1 | TGCAGCCAGGCGGTTCACTGAGACTTTCATGTGCTGCTTCAGGGTTTAC |
| | | CTTTTCATACTATGCTATGTCATGGGTACGACAAGCTCCAGGAAAACAAC |
| | | GCGAGTGGGTTTCAGCGATAGAAGGTAGCGGCGGCAGAACGTATTATG |
| | | CCGACTCTGTGAAGGGGCGATTCACGATTAGTCGAGACAATTCCAAGAA |
| | | CACTTTGTATCTGCAGATGAATAGCCTCCGCGCCGAAGATACGGCCGTG |
| | | TACTACTGCGTAAAAGAAACCGGCTGGGCAGACTTTGATTACTGGGGTC |
| | | AAGGCACACTGGTGACTGTATCATCTGGTGGTGGTGGTTCTGGAGGGG |
| | | GTGGGAGCGGAGGTGGAGGAAGTGAAGTTCAGTTGGTGGAGAGCGGT |
| | | GGAGGCCTTGTCCAGCCGGGTGGGTCTTTGCGGTTGAGTTGCGCTGCA |
| | | TCCGGTTTCACCTTCTCAGCCTATGATATGTCTTGGGTTAGGCAAGCTC |
| | | CGGGCAAACAATTGGAATGGGTGTCAGCAATATCCGGTTCAGGTGGGT |
| | | CCACATACTACGCGGATTCTGTTAAGGGACGATTTACCATTAGCAGAGA |
| | | CAATAGTAAAAACACTCTGTACTTGCAGATGAATAGTCTTCGCGCGGAG |
| | | GATACCGCCGTGTACTATTGTGCATATGATGGCGACAGCACGGGCTCTC |
| | | TTGACGAAATGGCCCACTGGGGACAAGGGACGTTGGTTACGGTGTCAA |
| | | GTACTACCACACCAGCTCCCAGGCCACCCACGCCTGCACCGACAATCG |
| | | CATCTCAACCACTCTCACTCCGCCCCGAAGCGTGCAGACCCGCAGCAG |
| | | GTGGAGCCGTACATACTAGGGGTCTTGACTTCGCCTGTGATATATATAT |
| | | TTGGGCGCCCTTGGCCGGGACTTGTGGGGTCCTTCTCCTGTCACTGGT |
| | | TATCACCCTTTACTGCAACCACAGGAACCGAAGACGTGTTTGCAAACGG |
| | | GGCAGAAAGAAACTCCTGTATATATTCAAACAACCATTTATGAGACCAGT |
| | | ACAAACTACTCAAGAGGAAGATGGCTGTAGCTGCCGATTTCCAGAAGAA |
| | | GAAGAAGGAGGATGTGAACTACGCGTTAAGTTCAGCAGGAGCGCAGAC |
| | | GCCCCCGCGTACCAGCAGGGCCAGAACCAGCTCTATAACGAGCTCAAT |
| | | CTAGGACGAAGAGAGGAGTACGATGTTTTGGACAAGCGCCGTGGCCGG |
| | | GACCCTGAGATGGGGGGAAAGCCGCAGAGAAGGAAGAACCCTCAGGA |
| | | AGGCCTGTACAATGAACTGCAGAAAGATAAGATGGCGGAGGCCTACAG |
| | | TGAGATTGGGATGAAAGGCGAGCGCCGGAGGGGCAAGGGGCACGATG |
| | | GCCTTTACCAGGGTCTCAGTACAGCCACCAAGGACACCTACGACGCCC |
| | | TTCACATGCAGGCCCTGCCCCCTCGC |
| 98 | Tandem CAR comprising | ATGGCCCTTCCCGTTACCGCCTTACTCTTACCTCTTGCTTTATTACTGCA |
| | antibodies 3 and 5 nucleic acid | CGCGGCACGGCCTGAAGTTCAGTTGGTGGAGAGCGGTGGAGGCCTTG |
| | Tandem-3.2 | TCCAGCCGGGTGGGTCTTTGCGGTTGAGTTGCGCTGCATCCGGTTTCA |
| | | CCTTCTCAGCCTATGATATGTCTTGGGTTAGGCAAGCTCCGGGCAAACA |
| | | ATTGGAATGGGTGTCAGCAATATCCGGTTCAGGTGGGTCCACATACTAC |
| | | GCGGATTCTGTTAAGGGACGATTTACCATTAGCAGAGACAATAGTAAAA |
| | | ACACTCTGTACTTGCAGATGAATAGTCTTCGCGCGGAGGATACCGCCGT |
| | | GTACTATTGTGCATATGATGGCGACAGCACGGGCTCTCTTGACGAAATG |
| | | GCCCACTGGGGACAAGGGACGTTGGTTACGGTGTCAAGTGGTGGTGGT |
| | | GGTTCTGGAGGGGGTGGGAGCGGAGGTGGAGGAAGTGAAGTCCAGTT |
| | | GGTCGAATCCGGCGGTGGCCTCGTGCAGCCAGGCGGTTCACTGAGACT |
| | | TTCATGTGCTGCTTCAGGGTTTACCTTTTCATACTATGCTATGTCATGGG |
| | | TACGACAAGCTCCAGGAAAACAACGCGAGTGGGTTTCAGCGATAGAAG |
| | | GTAGCGGCGGCAGAACGTATTATGCCGACTCTGTGAAGGGGCGATTCA |
| | | CGATTAGTCGAGACAATTCCAAGAACACTTTGTATCTGCAGATGAATAGC |
| | | CTCCGCGCCGAAGATACGGCCGTGTACTACTGCGTAAAAGAAACCGGC |
| | | TGGGCAGACTTTGATTACTGGGGTCAAGGCACACTGGTGACTGTATCAT |
| | | CTACTACCACACCAGCTCCCAGGCCACCCACGCCTGCACCGACAATCG |
| | | CATCTCAACCACTCTCACTCCGCCCCGAAGCGTGCAGACCCGCAGCAG |
| | | GTGGAGCCGTACATACTAGGGGTCTTGACTTCGCCTGTGATATATATAT |
| | | TTGGGCGCCCTTGGCCGGGACTTGTGGGGTCCTTCTCCTGTCACTGGT |
| | | TATCACCCTTTACTGCAACCACAGGAACCGAAGACGTGTTTGCAAACGG |
| | | GGCAGAAAGAAACTCCTGTATATATTCAAACAACCATTTATGAGACCAGT |
| | | ACAAACTACTCAAGAGGAAGATGGCTGTAGCTGCCGATTTCCAGAAGAA |
| | | GAAGAAGGAGGATGTGAACTACGCGTTAAGTTCAGCAGGAGCGCAGAC |
| | | GCCCCCGCGTACCAGCAGGGCCAGAACCAGCTCTATAACGAGCTCAAT |
| | | CTAGGACGAAGAGAGGAGTACGATGTTTTGGACAAGCGCCGTGGCCGG |
| | | GACCCTGAGATGGGGGGAAAGCCGCAGAGAAGGAAGAACCCTCAGGA |
| | | AGGCCTGTACAATGAACTGCAGAAAGATAAGATGGCGGAGGCCTACAG |
| | | TGAGATTGGGATGAAAGGCGAGCGCCGGAGGGGCAAGGGGCACGATG |
| | | GCCTTTACCAGGGTCTCAGTACAGCCACCAAGGACACCTACGACGCCC |
| | | TTCACATGCAGGCCCTGCCCCCTCGC |
| 99 | Tandem CAR comprising | ATGGCCCTTCCCGTTACCGCCTTACTCTTACCTCTTGCTTTATTACTGCA |
| | antibodies 5 with linker 3 | CGCGGCACGGCCTGAAGTCCAACTCGTCGAAAGCGGGGGAGGCTTGG |
| | nucleic acid | TACAGCCTGGGGGGTCCCTGAGACTCTCCTGTGCAGCCTCTGGATTCA |
| | Tandem-3.3 | CCTTTAGCTACTATGCCATGAGCTGGGTCCGCCAGGCTCCAGGGAAGC |
| | | AGCGTGAGTGGGTCTCAGCTATTGAAGGTAGTGGTGGTCGGACATACT |
| | | ACGCAGACTCCGTGAAGGGCCGGTTCACCATCTCCAGAGACAATTCCA |
| | | AGAACACGCTGTATCTGCAAATGAACAGCCTGAGAGCCGAGGACACGG |
| | | CCGTATATTACTGTGTTAAAGAGACAGGATGGGCGGATTTTGACTATTG |
| | | GGGCCAGGGAACCCTCGTGACAGTTAGCAGCGGGGGAGGTGGTTCCG |
| | | AGGTGCAGCTGGTGGAGTCTGGGGGAGGCTTGGTACAGCCTGGGGGG |
| | | TCCCTGAGACTCTCCTGTGCAGCCTCTGGATTCACCTTTAGCGCGTATG |
| | | ATATGAGCTGGGTCCGCCAGGCTCCAGGGAAGCAGCTTGAGTGGGTCT |
| | | CAGCTATTAGCGGTAGTGGTGGTAGCACATACTACGCAGACTCCGTGAA |
| | | GGGCCGGTTCACCATCTCCAGAGACAATTCCAAGAACACGCTGTATCTG |
| | | CAAATGAACAGCCTGAGAGCCGAGGACACGGCCGTATATTACTGTGCC |
| | | TACGATGGTGATTCTACAGGGTCTCTGGATGAGATGGCGCACTGGGGC |
| | | CAGGGAACCCTGGTCACCGTTTCATCTACTACCACACCAGCTCCCAGGC |
| | | CACCCACGCCTGCACCGACAATCGCATCTCAACCACTCTCACTCCGCCC |
| | | CGAAGCGTGCAGACCCGCAGCAGGTGGAGCCGTACATACTAGGGGTCT |
| | | TGACTTCGCCTGTGATATATATATTTGGGCGCCCTTGGCCGGGACTTGT |
| | | GGGGTCCTTCTCCTGTCACTGGTTATCACCCTTTACTGCAACCACAGGA |
| | | ACCGAAGACGTGTTTGCAAACGGGGCAGAAAGAAACTCCTGTATATATT |
| | | CAAACAACCATTTATGAGACCAGTACAAACTACTCAAGAGGAAGATGGC |
| | | TGTAGCTGCCGATTTCCAGAAGAAGAAGAAGGAGGATGTGAACTACGC |
| | | GTTAAGTTCAGCAGGAGCGCAGACGCCCCCGCGTACCAGCAGGGCCA |
| | | GAACCAGCTCTATAACGAGCTCAATCTAGGACGAAGAGAGGAGTACGAT |
| | | GTTTTGGACAAGCGCCGTGGCCGGGACCCTGAGATGGGGGGAAAGCC |
| | | GCAGAGAAGGAAGAACCCTCAGGAAGGCCTGTACAATGAACTGCAGAA |
| | | AGATAAGATGGCGGAGGCCTACAGTGAGATTGGGATGAAAGGCGAGCG |
| | | CCGGAGGGGCAAGGGGCACGATGGCCTTTACCAGGGTCTCAGTACAG |
| | | CCACCAAGGACACCTACGACGCCCTTCACATGCAGGCCCTGCCCCCTC |
| | | GC |
| 100 | Parallel CAR comprising | ATGGCGCTCCCCGTGACCGCGTTGCTTCTCCCGCTGGCACTCCTGTTG |
| | antibodies 3 and 5 with GSG- | CATGCGGCACGACCGGAAGTTCAGCTGGTGGAGAGCGGAGGAGGCCT |
| | T2A self-cleaving peptide | CGTCCAACCTGGGGGCTCACTTAGGTTGTCTTGCGCGGCGAGCGGCTT |
| | nucleic acid | CACCTTTTCAGCATATGATATGAGTTGGGTAAGACAAGCCCCAGGAAAA |
| | Parallel-3.2 | CAGCTCGAATGGGTATCTGCTATAAGTGGATCCGGAGGGTCAACTTACT |
| | | ACGCGGACTCTGTTAAGGGGCGCTTTACAATTAGCCGCGACAACAGTAA |
| | | GAACACTTTGTACCTTCAGATGAACTCACTTCGGGCCGAGGACACAGCT |
| | | GTATATTACTGTGCATATGATGGGGATTCTACTGGCTCTCTGGATGAGAT |
| | | GGCCCACTGGGGTCAAGGCACACTCGTGACGGTTTCCTCAACCACCAC |
| | | GCCAGCGCCTAGACCCCCTACTCCGGCCCCTACAATAGCAAGTCAACC |
| | | CCTCTCTCTGAGACCTGAAGCCTGCCGACCCGCAGCGGGTGGAGCTGT |
| | | CCACACCCGCGGCCTGGATTTCGCATGTGACATATACATATGGGCGCC |
| | | CCTGGCAGGTACCTGCGGTGTTCTGTTGCTTAGCCTTGTAATTACGCTT |
| | | TACTGTAACCACAGAAATCGACGGAGGGTCTGTAAGCGCGGTCGGAAG |
| | | AAGCTCTTGTACATTTTCAAGCAGCCTTTTATGAGGCCGGTCCAAACGA |
| | | CACAAGAGGAAGACGGATGCTCATGTAGGTTTCCAGAGGAAGAGGAAG |
| | | GAGGCTGTGAACTTCGGGTCAAATTTAGTAGATCTGCGGATGCACCGG |
| | | CATATCAACAAGGGCAAAATCAACTGTACAACGAGCTCAATCTGGGTAG |
| | | GCGAGAAGAATATGACGTACTTGATAAGCGGCGGGGACGGGATCCTGA |
| | | AATGGGTGGTAAACCACAGCGCAGGAAAAACCCGCAGGAGGGGCTTTA |
| | | TAACGAGCTTCAAAAAGACAAAATGGCGGAGGCCTACTCCGAAATAGGC |
| | | ATGAAGGGCGAAAGACGACGCGGTAAGGGGCACGATGGCTTGTACCAA |
| | | GGGTTGTCCACCGCGACTAAAGACACATACGACGCTCTTCATATGCAGG |
| | | CGTTGCCGCCGAGGGGTTCAGGCGAAGGCCGGGGCAGTCTTTTGACAT |
| | | GCGGCGATGTTGAGGAGAACCCTGGACCCATGGCGCTTCCTGTAACTG |
| | | CACTGCTTCTCCCACTCGCATTGCTTCTGCATGCAGCCCGCCCCGAAGT |
| | | TCAACTCGTAGAATCCGGGGGAGGCTTGGTCCAGCCAGGTGGATCCCT |
| | | CCGACTCTCATGTGCCGCAAGTGGGTTTACGTTCAGTTATTACGCAATG |
| | | TCATGGGTGAGACAGGCACCAGGGAAACAGAGAGAATGGGTAAGCGCA |
| | | ATCGAGGGATCCGGAGGGCGGACCTACTACGCCGACTCCGTCAAGGG |
| | | ACGCTTCACGATTAGCAGGGACAACAGTAAGAACACCTTGTACCTGCAA |
| | | ATGAATTCACTTCGAGCTGAAGACACTGCCGTTTATTATTGCGTCAAGGA |
| | | AACGGGCTGGGCCGATTTTGATTATTGGGGACAGGGAACATTGGTCAC |
| | | GGTGTCATCAACCCAGCCGCAAGAGACTCAAGGGCCGCCCGCCAGGC |
| | | CGATAACGGTACAGCCCACTGAGGCGTGGCCACGAACCTCTCAGGGCC |
| | | CATCCACTAGGCCCGTCGAAGTACCTGGGGGGAGAGCCGTCGCTGCCA |
| | | TCCTGGGGCTCGGTCTGGTTTTGGGCTTGTTGGGTCCGCTTGCAATTTT |
| | | GTTGGCCTTGTACCTTCTGCGGAGAGACCAGCGGAAGCGGGGTAGGAA |
| | | AAAACTTCTGTATATATTCAAGCAACCCTTCATGCGCCCGGTGCAGACC |
| | | ACCCAAGAAGAGGATGGGTGCAGCTGCAGGTTTCCAGAGGAGGAGGAA |
| | | GGCGGCTGCGAGTTGCGGGTAAAGTTTTCAAGGTCAGCAGATGCTCCG |
| | | GCCTACCAGCAAGGACAGAACCAATTGTACAATGAGCTGAATTTGGGTC |
| | | GCCGAGAAGAATATGACGTCTTGGATAAGCGCAGAGGACGGGACCCAG |
| | | AAATGGGGGGGAAGCCACAGAGGAGAAAGAATCCACAGGAGGGACTGT |
| | | ATAATGAATTGCAGAAAGATAAAATGGCAGAAGCCTACAGTGAAATCGG |
| | | GATGAAGGGCGAAAGACGCAGAGGGAAGGGGCATGATGGTTTGTATCA |
| | | AGGGCTGTCAACTGCGACAAAGGACACGTATGATGCACTGCATATGCA |
| | | GGCTCTTCCCCCGAGG |
| 101 | Parallel CAR comprising | ATGGCTCTTCCCGTGACGGCACTCCTTCTGCCCTTGGCACTCCTGCTGC |
| | antibodies 3 and 5 with GSG- | ACGCGGCAAGACCAGAGGTGCAGCTTGTCGAGTCAGGGGGTGGCCTC |
| | P2A - GSG-T2A self-cleaving | GTTCAGCCGGGTGGTAGTCTCAGGCTGAGTTGTGCAGCTTCCGGGTTC |
| | peptide nucleic acid | ACTTTCTCAGCATACGATATGTCCTGGGTTAGGCAAGCACCGGGGAAAC |
| | Parallel-3.3 | AACTTGAGTGGGTTTCCGCTATTTCTGGCAGTGGTGGGTCCACGTATTA |
| | | TGCGGACTCAGTGAAAGGCCGCTTCACTATTAGCCGGGATAATTCCAAG |
| | | AATACGCTCTACCTGCAAATGAATAGTCTGAGAGCGGAGGATACGGCG |
| | | GTTTATTATTGTGCGTATGACGGTGACAGCACGGGTAGCCTGGACGAAA |
| | | TGGCACACTGGGGACAGGGGACCCTTGTGACCGTCAGTAGTACTACCA |
| | | CTCCTGCACCACGACCTCCCACACCAGCACCTACCATAGCTTCTCAACC |
| | | TCTCTCTCTTCGGCCAGAAGCTTGTCGCCCTGCGGCAGGTGGTGCTGT |
| | | ACATACACGAGGCCTCGATTTCGCTTGCGATATATATATTTGGGCACCT |
| | | CTCGCTGGGACGTGTGGGGTGCTTCTTCTTTCCCTCGTCATCACGTTGT |
| | | ACTGCAATCATCGCAATCGCCGGCGAGTGTGCAAACGGGGTCGAAAGA |
| | | AACTCCTCTACATTTTTAAGCAGCCTTTTATGAGGCCGGTACAGACGAC |
| | | GCAGGAGGAGGATGGGTGTTCTTGTAGATTCCCAGAGGAGGAGGAAGG |
| | | AGGTTGTGAACTTCGGGTCAAATTCTCTCGCTCAGCCGACGCACCGGC |
| | | CTACCAACAGGGGCAAAATCAATTGTATAATGAGTTGAATCTGGGCCGC |
| | | AGAGAAGAGTACGACGTGCTCGATAAGCGAAGGGGTAGAGATCCTGAA |
| | | ATGGGAGGGAAACCGCAGCGGAGGAAAAACCCCCAAGAGGGATTGTAC |
| | | AATGAGCTTCAGAAGGATAAAATGGCTGAAGCTTATTCCGAGATTGGAA |
| | | TGAAAGGAGAGCGGCGGCGCGGTAAAGGTCACGATGGCTTGTATCAGG |
| | | GACTGTCCACCGCAACGAAGGACACTTACGACGCCCTTCATATGCAAGC |
| | | ATTGCCTCCACGAGGATCAGGCGCTACCAATTTTTCTCTTCTCAAACAG |
| | | GCCGGGGACGTTGAGGAAAACCCTGGTCCAGGATCCGGAGAGGGTAG |
| | | AGGATCCCTCCTGACCTGCGGTGACGTGGAAGAGAACCCAGGTCCAAT |
| | | GGCTCTGCCAGTGACTGCCCTCCTTCTGCCTCTTGCACTGCTTCTTCAT |
| | | GCGGCAAGACCAGAGGTTCAGCTGGTTGAATCTGGTGGAGGATTGGTG |
| | | CAGCCAGGTGGATCTCTTCGGCTCAGTTGCGCCGCAAGCGGCTTCACA |
| | | TTTTCATACTACGCAATGTCTTGGGTGCGGCAAGCACCAGGGAAACAAC |
| | | GCGAATGGGTTAGCGCAATCGAGGGCAGTGGTGGTCGGACATATTACG |
| | | CAGACAGCGTTAAGGGAAGGTTCACCATAAGCCGAGACAATTCAAAGAA |
| | | TACACTGTACTTGCAAATGAATTCCCTTAGAGCGGAGGATACAGCGGTA |
| | | TACTACTGTGTCAAAGAGACCGGGTGGGCTGACTTTGACTATTGGGGCC |
| | | AGGGCACCCTGGTTACGGTATCAAGCACACAGCCTCAAGAGACCCAGG |
| | | GTCCCCCGGCGAGACCGATTACTGTGCAGCCTACTGAAGCGTGGCCAA |
| | | GAACATCTCAGGGACCCAGTACACGGCCGGTAGAGGTCCCCGGGGGC |
| | | AGAGCGGTCGCCGCTATCCTTGGACTGGGTTTGGTCTTGGGATTGCTC |
| | | GGGCCTTTGGCCATACTGCTTGCACTCTATCTGCTGAGAAGGGATCAGC |
| | | GAAAGCGAGGTCGGAAAAAGTTGCTCTACATCTTTAAGCAACCATTCAT |
| | | GCGGCCTGTACAAACAACCCAGGAGGAAGATGGTTGTAGTTGCCGCTT |
| | | TCCAGAAGAGGAAGAAGGTGGCTGCGAATTGCGGGTAAAGTTCTCACG |
| | | AAGCGCAGATGCGCCGGCTTATCAGCAAGGTCAAAATCAGTTGTATAAC |
| | | GAGTTGAATCTGGGAAGGCGAGAAGAGTATGATGTTTTGGACAAACGGA |
| | | GAGGTCGAGACCCAGAAATGGGCGGTAAGCCGCAAAGGAGGAAAAACC |
| | | CCCAGGAGGGGTTGTATAATGAATTGCAAAAGGACAAAATGGCTGAAGC |
| | | GTACAGTGAAATAGGAATGAAAGGGGAGAGAAGGCGGGGCAAGGGCC |
| | | ATGACGGACTGTATCAGGGTCTCTCCACCGCAACTAAGGATACATACGA |
| | | TGCTTTGCACATGCAGGCACTTCCGCCAAGA |
| 102 | Parallel CAR comprising | MALPVTALLLPLALLLHAARPEVQLVESGGGLVQPGGSLRLSCAASGFTFS |
| | Antibodies 1 and 4 +tCD34 | TYDMSWVRQAPGKQREWVSAISGSGGTTYYADSVKGRFTISRDNSKNTLY |
| | Parallel-1+tCD34 | LQMNSLRAEDTAVYYCVRGDDHWGQGTLVTVSSTTTPAPRPPTPAPTIAS |
| | | QPLSLRPEACRPAAGGAVHTRGLDFACDIYIWAPLAGTCGVLLLSLVITLYC |
| | | NHRNRRRVCKRGRKKLLYIFKQPFMRPVQTTQEEDGCSCRFPEEEEGGCE |
| | | LRVKFSRSADAPAYQQGQNQLYNELNLGRREEYDVLDKRRGRDPEMGGK |
| | | PQRRKNPQEGLYNELQKDKMAEAYSEIGMKGERRRGKGHDGLYQGLSTA |
| | | TKDTYDALHMQALPPREGRGSLLTCGDVEENPGPMALPVTALLLPLALLLH |
| | | AARPEVQLVESGGGLVQPGGSLRLSCAASGFTFSTYAMSWVRQAPGKQR |
| | | EWVSAIEGSGGSTYYADSVKGRFTISRDNSKNTLYLQMNSLRAEDTAVYYC |
| | | AVETGWADFDYWGQGTLVTVSSTQPQETQGPPARPITVQPTEAWPRTSQ |
| | | GPSTRPVEVPGGRAVAAILGLGLVLGLLGPLAILLALYLLRRDQRKRGRKKL |
| | | LYIFKQPFMRPVQTTQEEDGCSCRFPEEEEGGCELRVKFSRSADAPAYQQ |
| | | GQNQLYNELNLGRREEYDVLDKRRGRDPEMGGKPQRRKNPQEGLYNELQ |
| | | KDKMAEAYSEIGMKGERRRGKGHDGLYQGLSTATKDTYDALHMQALPPRA |
| | | RAKRSGSGEGRGSLITCGDVEENPGPMPRGWTALCLLSLLPSGFMSLDNN |
| | | GTATPELPTQGTFSNVSTNVSYQETTTPSTLGSTSLHPVSQHGNEATTNITE |
| | | TTVKFTSTSVITSVYGNTNSSVQSQTSVISTVFTTPANVSTPETTLKPSLSPG |
| | | NVSDLSTTSTSLATSPTKPYTSSSPILSDIKAEIKCSGIREVKLTQGICLEQNK |
| | | TSSCAEFKKDRGEGLARVLCGEEQADADAGAQVCSLLLAQSEVRPQCLLL |
| | | VLANRTEISSKLQLMKKHQSDLKKLGILDFTEQDVASHQSYSQKTLIALVTS |
| | | GALLAVLGITGYFLMNRRSWSPTGERLELEP |
| 103 | Parallel CAR comprising | MALPVTALLLPLALLLHAARPEVQLVESGGGLVQPGGSLRLSCAASGFTFS |
| | Antibodies 2 and 4 +tCD34 | NYDMSWVRQAPGKQLEWVSAITGSGGSTYYADSVKGRFTISRDNSKNTLY |
| | Parallel-2+tCD34 | LQMNSLRAEDTAVYYCAAGVDGDYWGQGTLVTVSSTTTPAPRPPTPAPTI |
| | | ASQPLSLRPEACRPAAGGAVHTRGLDFACDIYIWAPLAGTCGVLLLSLVITL |
| | | YCNHRNRRRVCKRGRKKLLYIFKQPFMRPVQTTQEEDGCSCRFPEEEEGG |
| | | CELRVKFSRSADAPAYQQGQNQLYNELNLGRREEYDVLDKRRGRDPEMG |
| | | GKPQRRKNPQEGLYNELQKDKMAEAYSEIGMKGERRRGKGHDGLYQGLS |
| | | TATKDTYDALHMQALPPREGRGSLLTCGDVEENPGPMALPVTALLLPLALL |
| | | LHAARPEVQLVESGGGLVQPGGSLRLSCAASGFTFSTYAMSWVRQAPGK |
| | | QREWVSAIEGSGGSTYYADSVKGRFTISRDNSKNTLYLQMNSLRAEDTAVY |
| | | YCAVETGWADFDYWGQGTLVTVSSTQPQETQGPPARPITVQPTEAWPRT |
| | | SQGPSTRPVEVPGGRAVAAILGLGLVLGLLGPLAILLALYLLRRDQRKRGRK |
| | | KLLYIFKQPFMRPVQTTQEEDGCSCRFPEEEEGGCELRVKFSRSADAPAY |
| | | QQGQNQLYNELNLGRREEYDVLDKRRGRDPEMGGKPQRRKNPQEGLYN |
| | | ELQKDKMAEAYSEIGMKGERRRGKGHDGLYQGLSTATKDTYDALHMQALP |
| | | PRARAKRSGSGEGRGSLITCGDVEENPGPMPRGWTALCLLSLLPSGFMSL |
| | | DNNGTATPELPTQGTFSNVSTNVSYQETTTPSTLGSTSLHPVSQHGNEATT |
| | | NITETTVKFTSTSVITSVYGNTNSSVQSQTSVISTVFTTPANVSTPETTLKPSL |
| | | SPGNVSDLSTTSTSLATSPTKPYTSSSPILSDIKAEIKCSGIREVKLTQGICLE |
| | | QNKTSSCAEFKKDRGEGLARVLCGEEQADADAGAQVCSLLLAQSEVRPQC |
| | | LLLVLANRTEISSKLQLMKKHQSDLKKLGILDFTEQDVASHQSYSQKTLIALV |
| | | TSGALLAVLGITGYFLMNRRSWSPTGERLELEP |
| 104 | Parallel CAR comprising | MALPVTALLLPLALLLHAARPEVQLVESGGGLVQPGGSLRLSCAASGFTFS |
| | Antibodies 3 and 5 +tCD34 | AYDMSWVRQAPGKQLEWVSAISGSGGSTYYADSVKGRFTISRDNSKNTLY |
| | Parallel-3+tCD34 | LQMNSLRAEDTAVYYCAYDGDSTGSLDEMAHWGQGTLVTVSSTTTPAPRP |
| | | PTPAPTIASQPLSLRPEACRPAAGGAVHTRGLDFACDIYIWAPLAGTCGVLL |
| | | LSLVITLYCNHRNRRRVCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPQRRKNPQEGLYNELQKDKMAEAYSEIGMKGERRRGKGHD |
| | | GLYQGLSTATKDTYDALHMQALPPREGRGSLLTCGDVEENPGPMALPVTA |
| | | LLLPLALLLHAARPEVQLVESGGGLVQPGGSLRLSCAASGFTFSYYAMSWV |
| | | RQAPGKQREWVSAIEGSGGRTYYADSVKGRFTISRDNSKNTLYLQMNSLR |
| | | AEDTAVYYCVKETGWADFDYWGQGTLVTVSSTQPQETQGPPARPITVQPT |
| | | EAWPRTSQGPSTRPVEVPGGRAVAAILGLGLVLGLLGPLAILLALYLLRRDQ |
| | | RKRGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPQRRKNP |
| | | QEGLYNELQKDKMAEAYSEIGMKGERRRGKGHDGLYQGLSTATKDTYDAL |
| | | HMQALPPRARAKRSGSGEGRGSLITCGDVEENPGPMPRGWTALCLLSLLP |
| | | SGFMSLDNNGTATPELPTQGTFSNVSTNVSYQETTTPSTLGSTSLHPVSQH |
| | | GNEATTNITETTVKFTSTSVITSVYGNTNSSVQSQTSVISTVFTTPANVSTPE |
| | | TTLKPSLSPGNVSDLSTTSTSLATSPTKPYTSSSPILSDIKAEIKCSGIREVKL |
| | | TQGICLEQNKTSSCAEFKKDRGEGLARVLCGEEQADADAGAQVCSLLLAQ |
| | | SEVRPQCLLLVLANRTEISSKLQLMKKHQSDLKKLGILDFTEQDVASHQSYS |
| | | QKTLIALVTSGALLAVLGITGYFLMNRRSWSPTGERLELEP |
| 105 | Parallel CAR comprising | MALPVTALLLPLALLLHAARPEVQLVESGGGLVQPGGSLRLSCAASGFTFS |
| | Antibodies 3 and 5 +tCD34 | AYDMSWVRQAPGKQLEWVSAISGSGGSTYYADSVKGRFTISRDNSKNTLY |
| | Parallel-3.2+tCD34 | LQMNSLRAEDTAVYYCAYDGDSTGSLDEMAHWGQGTLVTVSSTTTPAPRP |
| | | PTPAPTIASQPLSLRPEACRPAAGGAVHTRGLDFACDIYIWAPLAGTCGVLL |
| | | LSLVITLYCNHRNRRRVCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPQRRKNPQEGLYNELQKDKMAEAYSEIGMKGERRRGKGHD |
| | | GLYQGLSTATKDTYDALHMQALPPRGSGEGRGSLLTCGDVEENPGPMALP |
| | | VTALLLPLALLLHAARPEVQLVESGGGLVQPGGSLRLSCAASGFTFSYYAM |
| | | SWVRQAPGKQREWVSAIEGSGGRTYYADSVKGRFTISRDNSKNTLYLQMN |
| | | SLRAEDTAVYYCVKETGWADFDYWGQGTLVTVSSTQPQETQGPPARPITV |
| | | QPTEAWPRTSQGPSTRPVEVPGGRAVAAILGLGLVLGLLGPLAILLALYLLR |
| | | RDQRKRGRKKLLYIFKQPFMRPVQTTQEEDGCSCRFPEEEEGGCELRVKF |
| | | SRSADAPAYQQGQNQLYNELNLGRREEYDVLDKRRGRDPEMGGKPQRR |
| | | KNPQEGLYNELQKDKMAEAYSEIGMKGERRRGKGHDGLYQGLSTATKDTY |
| | | DALHMQALPPRGSGATNFSLLKQAGDVEENPGPMPRGWTALCLLSLLPSG |
| | | FMSLDNNGTATPELPTQGTFSNVSTNVSYQETTTPSTLGSTSLHPVSQHGN |
| | | EATTNITETTVKFTSTSVITSVYGNTNSSVQSQTSVISTVFTTPANVSTPETTL |
| | | KPSLSPGNVSDLSTTSTSLATSPTKPYTSSSPILSDIKAEIKCSGIREVKLTQG |
| | | ICLEQNKTSSCAEFKKDRGEGLARVLCGEEQADADAGAQVCSLLLAQSEV |
| | | RPQCLLLVLANRTEISSKLQLMKKHQSDLKKLGILDFTEQDVASHQSYSQKT |
| | | LIALVTSGALLAVLGITGYFLMNRRSWSPTGERLELEP |
| 106 | Parallel CAR comprising | MALPVTALLLPLALLLHAARPEVQLVESGGGLVQPGGSLRLSCAASGFTFS |
| | Antibodies 3 and 5 +tCD34 | AYDMSWVRQAPGKQLEWVSAISGSGGSTYYADSVKGRFTISRDNSKNTLY |
| | Parallel-3.3+tCD34 | LQMNSLRAEDTAVYYCAYDGDSTGSLDEMAHWGQGTLVTVSSTTTPAPRP |
| | | PTPAPTIASQPLSLRPEACRPAAGGAVHTRGLDFACDIYIWAPLAGTCGVLL |
| | | LSLVITLYCNHRNRRRVCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPQRRKNPQEGLYNELQKDKMAEAYSEIGMKGERRRGKGHD |
| | | GLYQGLSTATKDTYDALHMQALPPRGSGATNFSLLKQAGDVEENPGPGSG |
| | | EGRGSLLTCGDVEENPGPMALPVTALLLPLALLLHAARPEVQLVESGGGLV |
| | | QPGGSLRLSCAASGFTFSYYAMSWVRQAPGKQREWVSAIEGSGGRTYYA |
| | | DSVKGRFTISRDNSKNTLYLQMNSLRAEDTAVYYCVKETGWADFDYWGQ |
| | | GTLVTVSSTQPQETQGPPARPITVQPTEAWPRTSQGPSTRPVEVPGGRAV |
| | | AAILGLGLVLGLLGPLAILLALYLLRRDQRKRGRKKLLYIFKQPFMRPVQTTQ |
| | | EEDGCSCRFPEEEEGGCELRVKFSRSADAPAYQQGQNQLYNELNLGRRE |
| | | EYDVLDKRRGRDPEMGGKPQRRKNPQEGLYNELQKDKMAEAYSEIGMKG |
| | | ERRRGKGHDGLYQGLSTATKDTYDALHMQALPPRGSGATNFSLLKQAGDV |
| | | EENPGPGSGEGRGSLLTCGDVEENPGPMPRGWTALCLLSLLPSGFMSLD |
| | | NNGTATPELPTQGTFSNVSTNVSYQETTTPSTLGSTSLHPVSQHGNEATTN |
| | | ITETTVKFTSTSVITSVYGNTNSSVQSQTSVISTVFTTPANVSTPETTLKPSLS |
| | | PGNVSDLSTTSTSLATSPTKPYTSSSPILSDIKAEIKCSGIREVKLTQGICLEQ |
| | | NKTSSCAEFKKDRGEGLARVLCGEEQADADAGAQVCSLLLAQSEVRPQCL |
| | | LLVLANRTEISSKLQLMKKHQSDLKKLGILDFTEQDVASHQSYSQKTLIALVT |
| | | SGALLAVLGITGYFLMNRRSWSPTGERLELEP |
| 107 | Parallel CAR comprising | ATGGCCCTTCCCGTTACCGCCTTACTCTTACCTCTTGCTTTATTACTGCA |
| | Antibodies 1 and 4 +tCD34 | CGCGGCACGGCCTGAAGTCCAACTCGTCGAAAGCGGGGGAGGCTTGG |
| | nucleic acid | TACAGCCTGGGGGGTCCCTGAGACTCTCCTGTGCAGCCTCTGGATTCA |
| | Parallel-1+tCD34 | CCTTTAGCACATATGATATGAGCTGGGTCCGCCAGGCTCCAGGGAAGC |
| | | AGCGTGAGTGGGTCTCAGCTATTTCCGGTAGTGGTGGTACAACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGTGCGCGGCGACGACCATTGGGGCCAGGGAACCCT |
| | | CGTGACAGTTAGCAGCACTACGACTCCTGCACCGCGACCGCCCACACC |
| | | AGCTCCTACCATAGCTTCCCAACCTCTCTCTCTTCGCCCAGAAGCGTGT |
| | | CGGCCTGCCGCGGGTGGCGCAGTACACACGAGGGGCCTGGACTTTGC |
| | | ATGTGATATCTACATCTGGGCTCCACTCGCAGGAACCTGTGGCGTATTG |
| | | CTTCTTTCCTTGGTGATTACTCTCTATTGCAATCACAGAAATAGGAGGCG |
| | | AGTGTGTAAACGCGGGCGCAAGAAGTTGTTGTACATATTCAAGCAACCC |
| | | TTTATGAGACCAGTGCAAACAACACAAGAAGAGGATGGTTGTTCATGCA |
| | | GATTCCCTGAGGAGGAAGAGGGCGGCTGCGAACTTAGGGTCAAGTTTT |
| | | CAAGGTCCGCTGACGCACCGGCATACCAGCAAGGCCAAAATCAGCTTT |
| | | ATAATGAACTGAATCTCGGGCGCCGAGAAGAATATGATGTTCTTGACAA |
| | | GAGGCGCGGGAGGGACCCCGAAATGGGGGGCAAACCCCAGCGGAGG |
| | | AAGAATCCCCAGGAAGGCCTTTACAATGAATTGCAGAAAGACAAAATGG |
| | | CTGAAGCATACAGTGAAATAGGAATGAAGGGGGAGAGGCGCCGCGGG |
| | | AAGGGACATGACGGATTGTATCAGGGATTGAGTACTGCGACGAAAGAC |
| | | ACGTACGATGCTCTTCACATGCAAGCGCTTCCACCGCGGGAGGGCAGA |
| | | GGAAGTCTTCTAACATGCGGTGACGTGGAGGAGAATCCCGGCCCTATG |
| | | GCCCTTCCCGTGACGGCTCTTCTTTTGCCACTGGCACTTCTTTTGCACG |
| | | CAGCGCGACCTGAGGTGCAGCTGGTGGAGTCTGGGGGAGGCTTGGTA |
| | | CAGCCTGGGGGGTCCCTGAGACTCTCCTGTGCAGCCTCTGGATTCACC |
| | | TTTAGCACGTATGCCATGAGCTGGGTCCGCCAGGCTCCAGGGAAGCAG |
| | | CGTGAGTGGGTCTCAGCTATTGAAGGTAGTGGTGGTTCTACATACTACG |
| | | CAGACTCCGTGAAGGGCCGGTTCACCATCTCCAGAGACAATTCCAAGAA |
| | | CACGCTGTATCTGCAAATGAACAGCCTGAGAGCCGAGGACACGGCCGT |
| | | ATATTACTGTGCCGTAGAGACAGGATGGGCGGATTTTGACTATTGGGGC |
| | | CAGGGAACCCTGGTCACCGTTTCATCTACGCAGCCCCAGGAGACCCAG |
| | | GGCCCCCCGGCCAGGCCCATCACTGTCCAGCCCACTGAAGCCTGGCC |
| | | CAGAACCTCACAGGGACCCTCCACCCGGCCCGTGGAGGTCCCCGGGG |
| | | GCCGTGCGGTTGCCGCCATCCTGGGCCTGGGCCTGGTGCTGGGGCTG |
| | | CTGGGCCCCCTGGCCATCCTGCTGGCCCTGTACCTGCTGCGGAGGGA |
| | | CCAGAGGAAACGGGGCAGAAAGAAACTCCTGTATATATTCAAACAACCA |
| | | TTTATGAGACCAGTACAAACTACTCAAGAGGAAGATGGCTGTAGCTGCC |
| | | GATTTCCAGAAGAAGAAGAAGGAGGATGTGAACTACGCGTTAAGTTCAG |
| | | CAGGAGCGCAGACGCCCCCGCGTACCAGCAGGGCCAGAACCAGCTCT |
| | | ATAACGAGCTCAATCTAGGACGAAGAGAGGAGTACGATGTTTTGGACAA |
| | | GCGCCGTGGCCGGGACCCTGAGATGGGGGGAAAGCCGCAGAGAAGGA |
| | | AGAACCCTCAGGAAGGCCTGTACAATGAACTGCAGAAAGATAAGATGGC |
| | | GGAGGCCTACAGTGAGATTGGGATGAAAGGCGAGCGCCGGAGGGGCA |
| | | AGGGGCACGATGGCCTTTACCAGGGTCTCAGTACAGCCACCAAGGACA |
| | | CCTACGACGCCCTTCACATGCAGGCCCTGCCCCCTCGCGCCCGCGCCA |
| | | AAAGGTCTGGCTCCGGTGAGGGCAGAGGAAGTCTTATAACATGCGGTG |
| | | ACGTGGAGGAGAATCCCGGCCCTATGCCTCGCGGCTGGACAGCCCTGT |
| | | GCCTGCTGTCTCTGCTGCCATCCGGCTTCATGAGCCTGGATAATAACGG |
| | | CACAGCCACCCCAGAGCTGCCTACACAGGGCACCTTCAGCAATGTGTC |
| | | CACAAACGTGAGCTATCAGGAGACCACAACCCCTTCTACCCTGGGATCT |
| | | ACAAGCCTGCACCCCGTGTCTCAGCACGGCAACGAAGCCACCACCAAC |
| | | ATCACCGAGACCACAGTGAAGTTTACCTCCACCTCTGTGATTACCTCTG |
| | | TGTACGGAAATACAAACTCCAGCGTGCAGTCTCAGACATCTGTGATCTC |
| | | CACAGTGTTTACAACACCTGCCAATGTGTCCACCCCAGAGACAACCCTG |
| | | AAGCCCAGCCTGTCTCCTGGAAATGTGTCCGATCTGTCTACCACCTCCA |
| | | CCAGCCTGGCCACCTCTCCCACCAAGCCCTATACCTCCTCTTCTCCCAT |
| | | CCTGAGCGATATCAAAGCCGAGATCAAATGCAGCGGGATTCGGGAAGT |
| | | GAAACTGACACAGGGCATCTGCCTGGAACAGAATAAGACATCCAGCTG |
| | | CGCCGAGTTTAAGAAAGATAGAGGAGAGGGACTGGCCAGGGTGCTGTG |
| | | TGGCGAAGAGCAGGCCGACGCCGATGCCGGCGCCCAGGTGTGTTCCC |
| | | TGCTGCTGGCCCAGTCTGAGGTGCGCCCCCAGTGCCTGCTGCTGGTGC |
| | | TGGCCAATCGGACAGAAATTAGCAGCAAGCTGCAGCTGATGAAAAAACA |
| | | CCAGAGCGATCTGAAAAAGCTGGGCATCCTGGACTTTACCGAGCAGGA |
| | | CGTGGCCTCTCACCAGAGCTACAGCCAGAAAACACTGATCGCCCTGGT |
| | | GACCAGCGGAGCCCTGCTGGCCGTGCTGGGCATCACCGGATATTTCCT |
| | | GATGAATAGGCGCAGCTGGAGCCCCACCGGCGAGCGGCTGGAGCTGG |
| | | AGCCT |
| 108 | Parallel CAR comprising | ATGGCCCTTCCCGTTACCGCCTTACTCTTACCTCTTGCTTTATTACTGCA |
| | Antibodies 2 and 4 +tCD34 | CGCGGCACGGCCTGAAGTCCAACTCGTCGAAAGCGGGGGAGGCTTGG |
| | nucleic acid | TACAGCCTGGGGGGTCCCTGAGACTCTCCTGTGCAGCCTCTGGATTCA |
| | Parallel-2+tCD34 | CCTTTAGCAACTATGACATGAGCTGGGTCCGCCAGGCTCCAGGGAAGC |
| | | AGCTTGAGTGGGTCTCAGCTATTACCGGTAGTGGTGGTAGTACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGCTGCAGGCGTAGATGGCGACTATTGGGGCCAGGG |
| | | AACCCTCGTGACAGTTAGCAGCACTACGACTCCTGCACCGCGACCGCC |
| | | CACACCAGCTCCTACCATAGCTTCCCAACCTCTCTCTCTTCGCCCAGAA |
| | | GCGTGTCGGCCTGCCGCGGGTGGCGCAGTACACACGAGGGGCCTGGA |
| | | CTTTGCATGTGATATCTACATCTGGGCTCCACTCGCAGGAACCTGTGGC |
| | | GTATTGCTTCTTTCCTTGGTGATTACTCTCTATTGCAATCACAGAAATAG |
| | | GAGGCGAGTGTGTAAACGCGGGCGCAAGAAGTTGTTGTACATATTCAA |
| | | GCAACCCTTTATGAGACCAGTGCAAACAACACAAGAAGAGGATGGTTGT |
| | | TCATGCAGATTCCCTGAGGAGGAAGAGGGCGGCTGCGAACTTAGGGTC |
| | | AAGTTTTCAAGGTCCGCTGACGCACCGGCATACCAGCAAGGCCAAAATC |
| | | AGCTTTATAATGAACTGAATCTCGGGCGCCGAGAAGAATATGATGTTCTT |
| | | GACAAGAGGCGCGGGAGGGACCCCGAAATGGGGGGCAAACCCCAGCG |
| | | GAGGAAGAATCCCCAGGAAGGCCTTTACAATGAATTGCAGAAAGACAAA |
| | | ATGGCTGAAGCATACAGTGAAATAGGAATGAAGGGGGAGAGGCGCCGC |
| | | GGGAAGGGACATGACGGATTGTATCAGGGATTGAGTACTGCGACGAAA |
| | | GACACGTACGATGCTCTTCACATGCAAGCGCTTCCACCGCGGGAGGGC |
| | | AGAGGAAGTCTTCTAACATGCGGTGACGTGGAGGAGAATCCCGGCCCT |
| | | ATGGCCCTTCCCGTGACGGCTCTTCTTTTGCCACTGGCACTTCTTTTGC |
| | | ACGCAGCGCGACCTGAGGTGCAGCTGGTGGAGTCTGGGGGAGGCTTG |
| | | GTACAGCCTGGGGGGTCCCTGAGACTCTCCTGTGCAGCCTCTGGATTC |
| | | ACCTTTAGCACGTATGCCATGAGCTGGGTCCGCCAGGCTCCAGGGAAG |
| | | CAGCGTGAGTGGGTCTCAGCTATTGAAGGTAGTGGTGGTTCTACATACT |
| | | ACGCAGACTCCGTGAAGGGCCGGTTCACCATCTCCAGAGACAATTCCA |
| | | AGAACACGCTGTATCTGCAAATGAACAGCCTGAGAGCCGAGGACACGG |
| | | CCGTATATTACTGTGCCGTAGAGACAGGATGGGCGGATTTTGACTATTG |
| | | GGGCCAGGGAACCCTGGTCACCGTTTCATCTACGCAGCCCCAGGAGAC |
| | | CCAGGGCCCCCCGGCCAGGCCCATCACTGTCCAGCCCACTGAAGCCT |
| | | GGCCCAGAACCTCACAGGGACCCTCCACCCGGCCCGTGGAGGTCCCC |
| | | GGGGGCCGTGCGGTTGCCGCCATCCTGGGCCTGGGCCTGGTGCTGGG |
| | | GCTGCTGGGCCCCCTGGCCATCCTGCTGGCCCTGTACCTGCTGCGGAG |
| | | GGACCAGAGGAAACGGGGCAGAAAGAAACTCCTGTATATATTCAAACAA |
| | | CCATTTATGAGACCAGTACAAACTACTCAAGAGGAAGATGGCTGTAGCT |
| | | GCCGATTTCCAGAAGAAGAAGAAGGAGGATGTGAACTACGCGTTAAGTT |
| | | CAGCAGGAGCGCAGACGCCCCCGCGTACCAGCAGGGCCAGAACCAGC |
| | | TCTATAACGAGCTCAATCTAGGACGAAGAGAGGAGTACGATGTTTTGGA |
| | | CAAGCGCCGTGGCCGGGACCCTGAGATGGGGGGAAAGCCGCAGAGAA |
| | | GGAAGAACCCTCAGGAAGGCCTGTACAATGAACTGCAGAAAGATAAGAT |
| | | GGCGGAGGCCTACAGTGAGATTGGGATGAAAGGCGAGCGCCGGAGGG |
| | | GCAAGGGGCACGATGGCCTTTACCAGGGTCTCAGTACAGCCACCAAGG |
| | | ACACCTACGACGCCCTTCACATGCAGGCCCTGCCCCCTCGCGCCCGCG |
| | | CCAAAAGGTCTGGCTCCGGTGAGGGCAGAGGAAGTCTTATAACATGCG |
| | | GTGACGTGGAGGAGAATCCCGGCCCTATGCCTCGCGGCTGGACAGCC |
| | | CTGTGCCTGCTGTCTCTGCTGCCATCCGGCTTCATGAGCCTGGATAATA |
| | | ACGGCACAGCCACCCCAGAGCTGCCTACACAGGGCACCTTCAGCAATG |
| | | TGTCCACAAACGTGAGCTATCAGGAGACCACAACCCCTTCTACCCTGGG |
| | | ATCTACAAGCCTGCACCCCGTGTCTCAGCACGGCAACGAAGCCACCAC |
| | | CAACATCACCGAGACCACAGTGAAGTTTACCTCCACCTCTGTGATTACC |
| | | TCTGTGTACGGAAATACAAACTCCAGCGTGCAGTCTCAGACATCTGTGA |
| | | TCTCCACAGTGTTTACAACACCTGCCAATGTGTCCACCCCAGAGACAAC |
| | | CCTGAAGCCCAGCCTGTCTCCTGGAAATGTGTCCGATCTGTCTACCACC |
| | | TCCACCAGCCTGGCCACCTCTCCCACCAAGCCCTATACCTCCTCTTCTC |
| | | CCATCCTGAGCGATATCAAAGCCGAGATCAAATGCAGCGGGATTCGGG |
| | | AAGTGAAACTGACACAGGGCATCTGCCTGGAACAGAATAAGACATCCAG |
| | | CTGCGCCGAGTTTAAGAAAGATAGAGGAGAGGGACTGGCCAGGGTGCT |
| | | GTGTGGCGAAGAGCAGGCCGACGCCGATGCCGGCGCCCAGGTGTGTT |
| | | CCCTGCTGCTGGCCCAGTCTGAGGTGCGCCCCCAGTGCCTGCTGCTGG |
| | | TGCTGGCCAATCGGACAGAAATTAGCAGCAAGCTGCAGCTGATGAAAAA |
| | | ACACCAGAGCGATCTGAAAAAGCTGGGCATCCTGGACTTTACCGAGCA |
| | | GGACGTGGCCTCTCACCAGAGCTACAGCCAGAAAACACTGATCGCCCT |
| | | GGTGACCAGCGGAGCCCTGCTGGCCGTGCTGGGCATCACCGGATATTT |
| | | CCTGATGAATAGGCGCAGCTGGAGCCCCACCGGCGAGCGGCTGGAGC |
| | | TGGAGCCT |
| 109 | Parallel CAR comprising | ATGGCCCTTCCCGTTACCGCCTTACTCTTACCTCTTGCTTTATTACTGCA |
| | Antibodies 3 and 5 +tCD34 | CGCGGCACGGCCTGAGGTGCAGCTGGTGGAGTCTGGGGGAGGCTTGG |
| | nucleic acid | TACAGCCTGGGGGGTCCCTGAGACTCTCCTGTGCAGCCTCTGGATTCA |
| | Parallel-3+tCD34 | CCTTTAGCGCGTATGATATGAGCTGGGTCCGCCAGGCTCCAGGGAAGC |
| | | AGCTTGAGTGGGTCTCAGCTATTAGCGGTAGTGGTGGTAGCACATACTA |
| | | CGCAGACTCCGTGAAGGGCCGGTTCACCATCTCCAGAGACAATTCCAA |
| | | GAACACGCTGTATCTGCAAATGAACAGCCTGAGAGCCGAGGACACGGC |
| | | CGTATATTACTGTGCCTACGATGGTGATTCTACAGGGTCTCTGGATGAG |
| | | ATGGCGCACTGGGGCCAGGGAACCCTGGTCACCGTTTCATCTACTACG |
| | | ACTCCTGCACCGCGACCGCCCACACCAGCTCCTACCATAGCTTCCCAA |
| | | CCTCTCTCTCTTCGCCCAGAAGCGTGTCGGCCTGCCGCGGGTGGCGCA |
| | | GTACACACGAGGGGCCTGGACTTTGCATGTGATATCTACATCTGGGCTC |
| | | CACTCGCAGGAACCTGTGGCGTATTGCTTCTTTCCTTGGTGATTACTCTC |
| | | TATTGCAATCACAGAAATAGGAGGCGAGTGTGTAAACGCGGGCGCAAG |
| | | AAGTTGTTGTACATATTCAAGCAACCCTTTATGAGACCAGTGCAAACAAC |
| | | ACAAGAAGAGGATGGTTGTTCATGCAGATTCCCTGAGGAGGAAGAGGG |
| | | CGGCTGCGAACTTAGGGTCAAGTTTTCAAGGTCCGCTGACGCACCGGC |
| | | ATACCAGCAAGGCCAAAATCAGCTTTATAATGAACTGAATCTCGGGCGC |
| | | CGAGAAGAATATGATGTTCTTGACAAGAGGCGCGGGAGGGACCCCGAA |
| | | ATGGGGGGCAAACCCCAGCGGAGGAAGAATCCCCAGGAAGGCCTTTAC |
| | | AATGAATTGCAGAAAGACAAAATGGCTGAAGCATACAGTGAAATAGGAA |
| | | TGAAGGGGGAGAGGCGCCGCGGGAAGGGACATGACGGATTGTATCAG |
| | | GGATTGAGTACTGCGACGAAAGACACGTACGATGCTCTTCACATGCAAG |
| | | CGCTTCCACCGCGGGAGGGCAGAGGAAGTCTTCTAACATGCGGTGACG |
| | | TGGAGGAGAATCCCGGCCCTATGGCCCTTCCCGTGACGGCTCTTCTTTT |
| | | GCCACTGGCACTTCTTTTGCACGCAGCGCGACCTGAAGTCCAACTCGTC |
| | | GAAAGCGGGGGAGGCTTGGTACAGCCTGGGGGGTCCCTGAGACTCTC |
| | | CTGTGCAGCCTCTGGATTCACCTTTAGCTACTATGCCATGAGCTGGGTC |
| | | CGCCAGGCTCCAGGGAAGCAGCGTGAGTGGGTCTCAGCTATTGAAGGT |
| | | AGTGGTGGTCGGACATACTACGCAGACTCCGTGAAGGGCCGGTTCACC |
| | | ATCTCCAGAGACAATTCCAAGAACACGCTGTATCTGCAAATGAACAGCC |
| | | TGAGAGCCGAGGACACGGCCGTATATTACTGTGTTAAAGAGACAGGAT |
| | | GGGCGGATTTTGACTATTGGGGCCAGGGAACCCTCGTGACAGTTAGCA |
| | | GCACGCAGCCCCAGGAGACCCAGGGCCCCCCGGCCAGGCCCATCACT |
| | | GTCCAGCCCACTGAAGCCTGGCCCAGAACCTCACAGGGACCCTCCACC |
| | | CGGCCCGTGGAGGTCCCCGGGGGCCGTGCGGTTGCCGCCATCCTGGG |
| | | CCTGGGCCTGGTGCTGGGGCTGCTGGGCCCCCTGGCCATCCTGCTGG |
| | | CCCTGTACCTGCTGCGGAGGGACCAGAGGAAACGGGGCAGAAAGAAA |
| | | CTCCTGTATATATTCAAACAACCATTTATGAGACCAGTACAAACTACTCA |
| | | AGAGGAAGATGGCTGTAGCTGCCGATTTCCAGAAGAAGAAGAAGGAGG |
| | | ATGTGAACTACGCGTTAAGTTCAGCAGGAGCGCAGACGCCCCCGCGTA |
| | | CCAGCAGGGCCAGAACCAGCTCTATAACGAGCTCAATCTAGGACGAAG |
| | | AGAGGAGTACGATGTTTTGGACAAGCGCCGTGGCCGGGACCCTGAGAT |
| | | GGGGGGAAAGCCGCAGAGAAGGAAGAACCCTCAGGAAGGCCTGTACA |
| | | ATGAACTGCAGAAAGATAAGATGGCGGAGGCCTACAGTGAGATTGGGA |
| | | TGAAAGGCGAGCGCCGGAGGGGCAAGGGGCACGATGGCCTTTACCAG |
| | | GGTCTCAGTACAGCCACCAAGGACACCTACGACGCCCTTCACATGCAG |
| | | GCCCTGCCCCCTCGCGCCCGCGCCAAAAGGTCTGGCTCCGGTGAGGG |
| | | CAGAGGAAGTCTTATAACATGCGGTGACGTGGAGGAGAATCCCGGCCC |
| | | TATGCCTCGCGGCTGGACAGCCCTGTGCCTGCTGTCTCTGCTGCCATC |
| | | CGGCTTCATGAGCCTGGATAATAACGGCACAGCCACCCCAGAGCTGCC |
| | | TACACAGGGCACCTTCAGCAATGTGTCCACAAACGTGAGCTATCAGGAG |
| | | ACCACAACCCCTTCTACCCTGGGATCTACAAGCCTGCACCCCGTGTCTC |
| | | AGCACGGCAACGAAGCCACCACCAACATCACCGAGACCACAGTGAAGT |
| | | TTACCTCCACCTCTGTGATTACCTCTGTGTACGGAAATACAAACTCCAGC |
| | | GTGCAGTCTCAGACATCTGTGATCTCCACAGTGTTTACAACACCTGCCA |
| | | ATGTGTCCACCCCAGAGACAACCCTGAAGCCCAGCCTGTCTCCTGGAA |
| | | ATGTGTCCGATCTGTCTACCACCTCCACCAGCCTGGCCACCTCTCCCAC |
| | | CAAGCCCTATACCTCCTCTTCTCCCATCCTGAGCGATATCAAAGCCGAG |
| | | ATCAAATGCAGCGGGATTCGGGAAGTGAAACTGACACAGGGCATCTGC |
| | | CTGGAACAGAATAAGACATCCAGCTGCGCCGAGTTTAAGAAAGATAGAG |
| | | GAGAGGGACTGGCCAGGGTGCTGTGTGGCGAAGAGCAGGCCGACGCC |
| | | GATGCCGGCGCCCAGGTGTGTTCCCTGCTGCTGGCCCAGTCTGAGGTG |
| | | CGCCCCCAGTGCCTGCTGCTGGTGCTGGCCAATCGGACAGAAATTAGC |
| | | AGCAAGCTGCAGCTGATGAAAAAACACCAGAGCGATCTGAAAAAGCTG |
| | | GGCATCCTGGACTTTACCGAGCAGGACGTGGCCTCTCACCAGAGCTAC |
| | | AGCCAGAAAACACTGATCGCCCTGGTGACCAGCGGAGCCCTGCTGGCC |
| | | GTGCTGGGCATCACCGGATATTTCCTGATGAATAGGCGCAGCTGGAGC |
| | | CCCACCGGCGAGCGGCTGGAGCTGGAGCCT |
| 110 | Parallel CAR comprising | ATGGCGCTCCCCGTGACCGCGTTGCTTCTCCCGCTGGCACTCCTGTTG |
| | Antibodies 3 and 5 +tCD34 | CATGCGGCACGACCGGAAGTTCAGCTGGTGGAGAGCGGAGGAGGCCT |
| | nucleic acid | CGTCCAACCTGGGGGCTCACTTAGGTTGTCTTGCGCGGCGAGCGGCTT |
| | Parallel-3.2+tCD34 | CACCTTTTCAGCATATGATATGAGTTGGGTAAGACAAGCCCCAGGAAAA |
| | | CAGCTCGAATGGGTATCTGCTATAAGTGGATCCGGAGGGTCAACTTACT |
| | | ACGCGGACTCTGTTAAGGGGCGCTTTACAATTAGCCGCGACAACAGTAA |
| | | GAACACTTTGTACCTTCAGATGAACTCACTTCGGGCCGAGGACACAGCT |
| | | GTATATTACTGTGCATATGATGGGGATTCTACTGGCTCTCTGGATGAGAT |
| | | GGCCCACTGGGGTCAAGGCACACTCGTGACGGTTTCCTCAACCACCAC |
| | | GCCAGCGCCTAGACCCCCTACTCCGGCCCCTACAATAGCAAGTCAACC |
| | | CCTCTCTCTGAGACCTGAAGCCTGCCGACCCGCAGCGGGTGGAGCTGT |
| | | CCACACCCGCGGCCTGGATTTCGCATGTGACATATACATATGGGCGCC |
| | | CCTGGCAGGTACCTGCGGTGTTCTGTTGCTTAGCCTTGTAATTACGCTT |
| | | TACTGTAACCACAGAAATCGACGGAGGGTCTGTAAGCGCGGTCGGAAG |
| | | AAGCTCTTGTACATTTTCAAGCAGCCTTTTATGAGGCCGGTCCAAACGA |
| | | CACAAGAGGAAGACGGATGCTCATGTAGGTTTCCAGAGGAAGAGGAAG |
| | | GAGGCTGTGAACTTCGGGTCAAATTTAGTAGATCTGCGGATGCACCGG |
| | | CATATCAACAAGGGCAAAATCAACTGTACAACGAGCTCAATCTGGGTAG |
| | | GCGAGAAGAATATGACGTACTTGATAAGCGGCGGGGACGGGATCCTGA |
| | | AATGGGTGGTAAACCACAGCGCAGGAAAAACCCGCAGGAGGGGCTTTA |
| | | TAACGAGCTTCAAAAAGACAAAATGGCGGAGGCCTACTCCGAAATAGGC |
| | | ATGAAGGGCGAAAGACGACGCGGTAAGGGGCACGATGGCTTGTACCAA |
| | | GGGTTGTCCACCGCGACTAAAGACACATACGACGCTCTTCATATGCAGG |
| | | CGTTGCCGCCGAGGGGTTCAGGCGAAGGCCGGGGCAGTCTTTTGACAT |
| | | GCGGCGATGTTGAGGAGAACCCTGGACCCATGGCGCTTCCTGTAACTG |
| | | CACTGCTTCTCCCACTCGCATTGCTTCTGCATGCAGCCCGCCCCGAAGT |
| | | TCAACTCGTAGAATCCGGGGGAGGCTTGGTCCAGCCAGGTGGATCCCT |
| | | CCGACTCTCATGTGCCGCAAGTGGGTTTACGTTCAGTTATTACGCAATG |
| | | TCATGGGTGAGACAGGCACCAGGGAAACAGAGAGAATGGGTAAGCGCA |
| | | ATCGAGGGATCCGGAGGGCGGACCTACTACGCCGACTCCGTCAAGGG |
| | | ACGCTTCACGATTAGCAGGGACAACAGTAAGAACACCTTGTACCTGCAA |
| | | ATGAATTCACTTCGAGCTGAAGACACTGCCGTTTATTATTGCGTCAAGGA |
| | | AACGGGCTGGGCCGATTTTGATTATTGGGGACAGGGAACATTGGTCAC |
| | | GGTGTCATCAACCCAGCCGCAAGAGACTCAAGGGCCGCCCGCCAGGC |
| | | CGATAACGGTACAGCCCACTGAGGCGTGGCCACGAACCTCTCAGGGCC |
| | | CATCCACTAGGCCCGTCGAAGTACCTGGGGGGAGAGCCGTCGCTGCCA |
| | | TCCTGGGGCTCGGTCTGGTTTTGGGCTTGTTGGGTCCGCTTGCAATTTT |
| | | GTTGGCCTTGTACCTTCTGCGGAGAGACCAGCGGAAGCGGGGTAGGAA |
| | | AAAACTTCTGTATATATTCAAGCAACCCTTCATGCGCCCGGTGCAGACC |
| | | ACCCAAGAAGAGGATGGGTGCAGCTGCAGGTTTCCAGAGGAGGAGGAA |
| | | GGCGGCTGCGAGTTGCGGGTAAAGTTTTCAAGGTCAGCAGATGCTCCG |
| | | GCCTACCAGCAAGGACAGAACCAATTGTACAATGAGCTGAATTTGGGTC |
| | | GCCGAGAAGAATATGACGTCTTGGATAAGCGCAGAGGACGGGACCCAG |
| | | AAATGGGGGGGAAGCCACAGAGGAGAAAGAATCCACAGGAGGGACTGT |
| | | ATAATGAATTGCAGAAAGATAAAATGGCAGAAGCCTACAGTGAAATCGG |
| | | GATGAAGGGCGAAAGACGCAGAGGGAAGGGGCATGATGGTTTGTATCA |
| | | AGGGCTGTCAACTGCGACAAAGGACACGTATGATGCACTGCATATGCA |
| | | GGCTCTTCCCCCGAGGGGCTCAGGTGCAACCAATTTCAGCCTTCTGAAA |
| | | CAGGCTGGGGATGTGGAAGAAAATCCCGGCCCGATGCCTCGCGGCTG |
| | | GACAGCCCTGTGCCTGCTGTCTCTGCTGCCATCCGGCTTCATGAGCCT |
| | | GGATAATAACGGCACAGCCACCCCAGAGCTGCCTACACAGGGCACCTT |
| | | CAGCAATGTGTCCACAAACGTGAGCTATCAGGAGACCACAACCCCTTCT |
| | | ACCCTGGGATCTACAAGCCTGCACCCCGTGTCTCAGCACGGCAACGAA |
| | | GCCACCACCAACATCACCGAGACCACAGTGAAGTTTACCTCCACCTCTG |
| | | TGATTACCTCTGTGTACGGAAATACAAACTCCAGCGTGCAGTCTCAGAC |
| | | ATCTGTGATCTCCACAGTGTTTACAACACCTGCCAATGTGTCCACCCCA |
| | | GAGACAACCCTGAAGCCCAGCCTGTCTCCTGGAAATGTGTCCGATCTGT |
| | | CTACCACCTCCACCAGCCTGGCCACCTCTCCCACCAAGCCCTATACCTC |
| | | CTCTTCTCCCATCCTGAGCGATATCAAAGCCGAGATCAAATGCAGCGGG |
| | | ATTCGGGAAGTGAAACTGACACAGGGCATCTGCCTGGAACAGAATAAGA |
| | | CATCCAGCTGCGCCGAGTTTAAGAAAGATAGAGGAGAGGGACTGGCCA |
| | | GGGTGCTGTGTGGCGAAGAGCAGGCCGACGCCGATGCCGGCGCCCAG |
| | | GTGTGTTCCCTGCTGCTGGCCCAGTCTGAGGTGCGCCCCCAGTGCCTG |
| | | CTGCTGGTGCTGGCCAATCGGACAGAAATTAGCAGCAAGCTGCAGCTG |
| | | ATGAAAAAACACCAGAGCGATCTGAAAAAGCTGGGCATCCTGGACTTTA |
| | | CCGAGCAGGACGTGGCCTCTCACCAGAGCTACAGCCAGAAAACACTGA |
| | | TCGCCCTGGTGACCAGCGGAGCCCTGCTGGCCGTGCTGGGCATCACC |
| | | GGATATTTCCTGATGAATAGGCGCAGCTGGAGCCCCACCGGCGAGCGG |
| | | CTGGAGCTGGAGCCT |
| 111 | Parallel CAR comprising | ATGGCTCTTCCCGTGACGGCACTCCTTCTGCCCTTGGCACTCCTGCTGC |
| | Antibodies 3 and 5 +tCD34 | ACGCGGCAAGACCAGAGGTGCAGCTTGTCGAGTCAGGGGGTGGCCTC |
| | nucleic acid | GTTCAGCCGGGTGGTAGTCTCAGGCTGAGTTGTGCAGCTTCCGGGTTC |
| | Parallel-3.3+tCD34 | ACTTTCTCAGCATACGATATGTCCTGGGTTAGGCAAGCACCGGGGAAAC |
| | | AACTTGAGTGGGTTTCCGCTATTTCTGGCAGTGGTGGGTCCACGTATTA |
| | | TGCGGACTCAGTGAAAGGCCGCTTCACTATTAGCCGGGATAATTCCAAG |
| | | AATACGCTCTACCTGCAAATGAATAGTCTGAGAGCGGAGGATACGGCG |
| | | GTTTATTATTGTGCGTATGACGGTGACAGCACGGGTAGCCTGGACGAAA |
| | | TGGCACACTGGGGACAGGGGACCCTTGTGACCGTCAGTAGTACTACCA |
| | | CTCCTGCACCACGACCTCCCACACCAGCACCTACCATAGCTTCTCAACC |
| | | TCTCTCTCTTCGGCCAGAAGCTTGTCGCCCTGCGGCAGGTGGTGCTGT |
| | | ACATACACGAGGCCTCGATTTCGCTTGCGATATATATATTTGGGCACCT |
| | | CTCGCTGGGACGTGTGGGGTGCTTCTTCTTTCCCTCGTCATCACGTTGT |
| | | ACTGCAATCATCGCAATCGCCGGCGAGTGTGCAAACGGGGTCGAAAGA |
| | | AACTCCTCTACATTTTTAAGCAGCCTTTTATGAGGCCGGTACAGACGAC |
| | | GCAGGAGGAGGATGGGTGTTCTTGTAGATTCCCAGAGGAGGAGGAAGG |
| | | AGGTTGTGAACTTCGGGTCAAATTCTCTCGCTCAGCCGACGCACCGGC |
| | | CTACCAACAGGGGCAAAATCAATTGTATAATGAGTTGAATCTGGGCCGC |
| | | AGAGAAGAGTACGACGTGCTCGATAAGCGAAGGGGTAGAGATCCTGAA |
| | | ATGGGAGGGAAACCGCAGCGGAGGAAAAACCCCCAAGAGGGATTGTAC |
| | | AATGAGCTTCAGAAGGATAAAATGGCTGAAGCTTATTCCGAGATTGGAA |
| | | TGAAAGGAGAGCGGCGGCGCGGTAAAGGTCACGATGGCTTGTATCAGG |
| | | GACTGTCCACCGCAACGAAGGACACTTACGACGCCCTTCATATGCAAGC |
| | | ATTGCCTCCACGAGGATCAGGCGCTACCAATTTTTCTCTTCTCAAACAG |
| | | GCCGGGGACGTTGAGGAAAACCCTGGTCCAGGATCCGGAGAGGGTAG |
| | | AGGATCCCTCCTGACCTGCGGTGACGTGGAAGAGAACCCAGGTCCAAT |
| | | GGCTCTGCCAGTGACTGCCCTCCTTCTGCCTCTTGCACTGCTTCTTCAT |
| | | GCGGCAAGACCAGAGGTTCAGCTGGTTGAATCTGGTGGAGGATTGGTG |
| | | CAGCCAGGTGGATCTCTTCGGCTCAGTTGCGCCGCAAGCGGCTTCACA |
| | | TTTTCATACTACGCAATGTCTTGGGTGCGGCAAGCACCAGGGAAACAAC |
| | | GCGAATGGGTTAGCGCAATCGAGGGCAGTGGTGGTCGGACATATTACG |
| | | CAGACAGCGTTAAGGGAAGGTTCACCATAAGCCGAGACAATTCAAAGAA |
| | | TACACTGTACTTGCAAATGAATTCCCTTAGAGCGGAGGATACAGCGGTA |
| | | TACTACTGTGTCAAAGAGACCGGGTGGGCTGACTTTGACTATTGGGGCC |
| | | AGGGCACCCTGGTTACGGTATCAAGCACACAGCCTCAAGAGACCCAGG |
| | | GTCCCCCGGCGAGACCGATTACTGTGCAGCCTACTGAAGCGTGGCCAA |
| | | GAACATCTCAGGGACCCAGTACACGGCCGGTAGAGGTCCCCGGGGGC |
| | | AGAGCGGTCGCCGCTATCCTTGGACTGGGTTTGGTCTTGGGATTGCTC |
| | | GGGCCTTTGGCCATACTGCTTGCACTCTATCTGCTGAGAAGGGATCAGC |
| | | GAAAGCGAGGTCGGAAAAAGTTGCTCTACATCTTTAAGCAACCATTCAT |
| | | GCGGCCTGTACAAACAACCCAGGAGGAAGATGGTTGTAGTTGCCGCTT |
| | | TCCAGAAGAGGAAGAAGGTGGCTGCGAATTGCGGGTAAAGTTCTCACG |
| | | AAGCGCAGATGCGCCGGCTTATCAGCAAGGTCAAAATCAGTTGTATAAC |
| | | GAGTTGAATCTGGGAAGGCGAGAAGAGTATGATGTTTTGGACAAACGGA |
| | | GAGGTCGAGACCCAGAAATGGGCGGTAAGCCGCAAAGGAGGAAAAACC |
| | | CCCAGGAGGGGTTGTATAATGAATTGCAAAAGGACAAAATGGCTGAAGC |
| | | GTACAGTGAAATAGGAATGAAAGGGGAGAGAAGGCGGGGCAAGGGCC |
| | | ATGACGGACTGTATCAGGGTCTCTCCACCGCAACTAAGGATACATACGA |
| | | TGCTTTGCACATGCAGGCACTTCCGCCAAGAGGCTCAGGAGCTACAAAT |
| | | TTCTCACTCTTGAAGCAAGCGGGAGATGTCGAAGAAAATCCTGGTCCGG |
| | | GTTCAGGCGAGGGACGCGGATCATTGCTCACCTGTGGGGATGTGGAAG |
| | | AGAACCCCGGTCCGATGCCTCGCGGCTGGACAGCCCTGTGCCTGCTGT |
| | | CTCTGCTGCCATCCGGCTTCATGAGCCTGGATAATAACGGCACAGCCAC |
| | | CCCAGAGCTGCCTACACAGGGCACCTTCAGCAATGTGTCCACAAACGT |
| | | GAGCTATCAGGAGACCACAACCCCTTCTACCCTGGGATCTACAAGCCTG |
| | | CACCCCGTGTCTCAGCACGGCAACGAAGCCACCACCAACATCACCGAG |
| | | ACCACAGTGAAGTTTACCTCCACCTCTGTGATTACCTCTGTGTACGGAA |
| | | ATACAAACTCCAGCGTGCAGTCTCAGACATCTGTGATCTCCACAGTGTT |
| | | TACAACACCTGCCAATGTGTCCACCCCAGAGACAACCCTGAAGCCCAG |
| | | CCTGTCTCCTGGAAATGTGTCCGATCTGTCTACCACCTCCACCAGCCTG |
| | | GCCACCTCTCCCACCAAGCCCTATACCTCCTCTTCTCCCATCCTGAGCG |
| | | ATATCAAAGCCGAGATCAAATGCAGCGGGATTCGGGAAGTGAAACTGAC |
| | | ACAGGGCATCTGCCTGGAACAGAATAAGACATCCAGCTGCGCCGAGTT |
| | | TAAGAAAGATAGAGGAGAGGGACTGGCCAGGGTGCTGTGTGGCGAAGA |
| | | GCAGGCCGACGCCGATGCCGGCGCCCAGGTGTGTTCCCTGCTGCTGG |
| | | CCCAGTCTGAGGTGCGCCCCCAGTGCCTGCTGCTGGTGCTGGCCAATC |
| | | GGACAGAAATTAGCAGCAAGCTGCAGCTGATGAAAAAACACCAGAGCG |
| | | ATCTGAAAAAGCTGGGCATCCTGGACTTTACCGAGCAGGACGTGGCCT |
| | | CTCACCAGAGCTACAGCCAGAAAACACTGATCGCCCTGGTGACCAGCG |
| | | GAGCCCTGCTGGCCGTGCTGGGCATCACCGGATATTTCCTGATGAATA |
| | | GGCGCAGCTGGAGCCCCACCGGCGAGCGGCTGGAGCTGGAGCCT |

1. An antigen binding molecule that binds CLL1 and/or CD33; wherein the antigen binding molecule comprises an antigen binding domain selected from the group consisting of:
Antigen binding domain 1, comprising a single variable domain on a heavy chain comprising,
(i) a CDR1 of SEQ ID NO:8,
(ii) a CDR2 of SEQ ID NO:11, and
(iii) a CDR3 of SEQ ID NO:14;
and,
Antigen binding domain 2, comprising a single variable domain on a heavy chain comprising,
(i) a CDR1 of SEQ ID NO:9,
(ii) a CDR2 of SEQ ID NO:12, and
(iii) a CDR3 of SEQ ID NO:15;
and,
Antigen binding domain 3, comprising a single variable domain on a heavy chain comprising,
(i) a CDR1 of SEQ ID NO:10,
(ii) a CDR2 of SEQ ID NO:13, and
(iii) a CDR3 of SEQ ID NO:16;
and,
Antigen binding domain 4, comprising a single variable domain on a heavy chain comprising,
(i) a CDR1 of SEQ ID NO:19,
(ii) a CDR2 of SEQ ID NO:21, and
(iii) a CDR3 of SEQ ID NO:23;
and,
Antigen binding domain 5, comprising a single variable domain on a heavy chain comprising,
(i) a CDR1 of SEQ ID NO:20,
(ii) a CDR2 of SEQ ID NO:22, and
(iii) a CDR3 of SEQ ID NO:24.

2. The antigen binding molecule of claim 1, wherein the antigen binding molecule binds to CLL1 and CD33.

3. The antigen binding molecule of claim 2, wherein the antigen binding molecule comprises one of antigen binding domain 1 to 3 and one of antigen binding domain 4 to 5.

4. The antigen binding molecule of any one of claims 1 to 3, wherein the antigen binding molecule comprises:
a. Antigen binding domain 1 and antigen binding domain 4; or
b. Antigen binding domain 2 and antigen binding domain 4; or
c. Antigen binding domain 3 and antigen binding domain 5.

5. The antigen binding molecule of any one of claims 1 to 4, wherein, the single variable domain on a heavy chain of any one of antigen binding domain 1 to 5 is a VHH.

6. The antigen binding molecule of claim 5, wherein the VHH is selected from the group consisting of: SEQ ID NOs 5 to 7 and SEQ ID NOs 17 to 18.

7. The antigen binding molecule of any one of claims 1 to 6, wherein the antigen binding molecule is a chimeric antigen receptor, wherein the chimeric antigen receptor further comprises a hinge domain, a transmembrane domain and an intracellular T-cell receptor signaling domain.

8. The antigen binding molecule of claim 7, wherein the hinge region and the transmembrane region is from CD8A or TNFRSF4.

9. The antigen binding molecule of claim 7 or claim 8, wherein the intracellular T-cell receptor signaling domain is from CD247 (CD3-zeta).

10. The antigen binding molecule of any one of claims 7 to 9, wherein the chimeric antigen receptor further comprises an intracellular domain of a costimulatory protein receptor.

11. The antigen binding molecule of claim 10, wherein the intracellular domain of a costimulatory protein receptor is from CD137.

12. The antigen binding molecule of any one of claims 1 to 6, wherein the antigen binding molecule is a bi-specific T-cell engager (BiTE) comprising a T-cell antigen binding domain.

13. The antigen binding molecule of claim 12, wherein the T-cell antigen binding domain comprises an antibody fragment that specifically binds CD3.

14. The antigen binding molecule of any one of claims 1 to 6, wherein the antigen binding molecule is an antibody-drug conjugate (ADC) comprising a cytotoxic drug or a therapeutic radioisotope.

15. A pharmaceutical composition comprising the antigen binding molecule of any one of claims 1 to 14 and a pharmaceutically acceptable carrier.

16. A chimeric antigen receptor that binds CLL1 and/or CD33 comprising one or more antigen binding domains selected from the group consisting of:
Antigen binding domain 1, comprising a single variable domain on a heavy chain comprising,
(i) a CDR1 of SEQ ID NO:8,
(ii) a CDR2 of SEQ ID NO:11, and
(iii) a CDR3 of SEQ ID NO:14;
and,
Antigen binding domain 2, comprising a single variable domain on a heavy chain comprising,
(i) a CDR1 of SEQ ID NO:9,
(ii) a CDR2 of SEQ ID NO:12, and
(iii) a CDR3 of SEQ ID NO:15;
and,
Antigen binding domain 3, comprising a single variable domain on a heavy chain comprising,
(i) a CDR1 of SEQ ID NO:10,
(ii) a CDR2 of SEQ ID NO:13, and
(iii) a CDR3 of SEQ ID NO:16;
and,
Antigen binding domain 4, comprising a single variable domain on a heavy chain comprising,
(i) a CDR1 of SEQ ID NO:19,
(ii) a CDR2 of SEQ ID NO:21, and
(iii) a CDR3 of SEQ ID NO:23;
and,
Antigen binding domain 5, comprising a single variable domain on a heavy chain comprising,
(i) a CDR1 of SEQ ID NO:20,
(ii) a CDR2 of SEQ ID NO:22, and
(iii) a CDR3 of SEQ ID NO:24;
b) A hinge domain;
c) A transmembrane domain; and
d) An intracellular signaling domain.

17. The chimeric antigen receptor of claim 16, wherein the hinge domain and the transmembrane region is from CD8A or TNFRSF4.

18. The chimeric antigen receptor of claim 16 or claim 17, wherein the intracellular signaling domain is from CD247 (CD3-zeta).

19. The chimeric antigen receptor of any one of claims 16 to 18, wherein the chimeric antigen receptor further comprises an intracellular signaling domain of a costimulatory protein receptor.

20. The chimeric antigen receptor of claim 19, wherein the intracellular signaling domain of a costimulatory protein receptor is from CD137.

21. The chimeric antigen receptor of any one of claims 16 to 20, wherein, the single variable domain on a heavy chain of any one of antigen binding domain 1 to 5 is a VHH.

22. The chimeric antigen receptor, wherein the VHH is selected from the group consisting of: SEQ ID NOs 5 to 7 and SEQ ID NOs 17 to 18.

23. The chimeric antigen receptor of any one of claims 16 to 22, wherein the CAR comprises:
a. a CD8A extracellular signal peptide (SEQ ID NO:39); one or more antigen binding domains (SEQ ID NOS: 5, 6, 7, 17 or 18); a human CD8A hinge/transmembrane domain (SEQ ID NO:34), a human CD137 co-stimulatory domain (SEQ ID NO:33); and a human CD247 (CD3-zeta) intracellular signaling domain (SEQ ID NO:32); or
b. a CD8A extracellular signal peptide (SEQ ID NO:39); one or more antigen binding domains of the antigen binding molecule of the first aspect (SEQ ID NOS: 5, 6, 7, 17 or 18); a human TNFRSF4 hinge/transmembrane domain (SEQ ID NO:35); a human CD137 co-stimulatory domain (SEQ ID NO:33); a human CD137 co-stimulatory domain (SEQ ID NO:33); and a human CD247 (CD3-zeta) intracellular signaling domain (SEQ ID NO: 32).

24. The chimeric antigen receptor of any one of claims 16 to 23, wherein the CAR has a sequence selected from the group consisting of: SEQ ID NOS: 25 to 31.

25. The chimeric antigen receptor of claims 16 to 24, wherein the chimeric antigen receptor binds CLL1 and CD33.

26. The chimeric antigen receptor of claim 25, wherein the chimeric antigen receptor comprises one of antigen binding domain 1 to 3 and one of antigen binding domain 4 to 5.

27. The chimeric antigen receptor of claim 25 or 26, comprising:
a. Antigen binding domain 1 and antigen binding domain 4; or
b. Antigen binding domain 2 and antigen binding domain 4; or
c. Antigen binding domain 3 and antigen binding domain 5.

28. The chimeric antigen receptor of any one of claims 25 to 27, wherein the CAR comprises two antigen binding domains joined by at least one linker.

29. The chimeric antigen receptor of claim 28, wherein the linker has a sequence selected from the group consisting of: SEQ ID NOS: 36 to 38.

30. The chimeric antigen receptor of claim 28 or claim 29, wherein the CAR comprises:
a. a CD8A extracellular signal peptide (SEQ ID NO: 39); a first antigen binding domain (SEQ ID NO: 5, 6 or 7); a linker (SEQ ID NO: 36, 37 or 38); a second antigen binding domain (SEQ ID NO: 17 or 18); a human CD8A hinge/transmembrane domain (SEQ ID NO: 34), a human CD137 co-stimulatory domain (SEQ ID NO: 33); and a human CD247 (CD3-zeta) intracellular signalling domain (SEQ ID NO: 32); or
b. a CD8A extracellular signal peptide (SEQ ID NO:39); a first antigen binding domain (SEQ ID NO: 17 or 18); a linker (SEQ ID NO: 36, 37 or 38); a second antigen binding domain (SEQ ID NO: 5, 6 or 7); a human CD8A hinge/transmembrane domain (SEQ ID NO: 34), a human CD137 co-stimulatory domain (SEQ ID NO: 33); and a human CD247 (CD3-zeta) intracellular signalling domain (SEQ ID NO: 32); or
c. a CD8A extracellular signal peptide (SEQ ID NO: 39); a first antigen binding domain (SEQ ID NO: 5, 6 or 7); a linker (SEQ ID NO: 36, 37 or 38); a second antigen binding domain (SEQ ID NO: 17 or 18); a human TNFRSF4 hinge/transmembrane domain (SEQ ID NO: 35); a human CD137 co-stimulatory domain (SEQ ID NO: 33); a human CD137 co-stimulatory domain (SEQ ID NO: 33); and a human CD247 (CD3-zeta) intracellular signalling domain (SEQ ID NO: 32); or
d. a CD8A extracellular signal peptide (SEQ ID NO:39); a first antigen binding domain (SEQ ID NO: 17 or 18); a linker (SEQ ID NO: 36 to 38); a second antigen binding domain (SEQ ID NO: 5, 6 or 7); a human TNFRSF4 hinge/transmembrane domain (SEQ ID NO: 35); a human CD137 co-stimulatory domain SEQ ID NO: 33); and a human CD247 (CD3-zeta) intracellular signalling domain (SEQ ID NO: 32).

31. The chimeric antigen receptor of any one of claims 28 to 30, wherein the CAR has a sequence selected from the group consisting of: SEQ ID NOS: 40 to 42; 93 and 94.

32. The chimeric antigen receptor of any one of claims 16 to 31, wherein the CAR is linked to a tCD34 domain by a second self-cleaving peptide.

33. The chimeric antigen receptor of claim 32, wherein the second self-cleaving peptide has a sequence of SEQ ID NO. 90 and tCD34 has the sequence SEQ ID NO. 92.

34. The chimeric antigen receptor of any one of claims 16 to 27, wherein the CAR is encoded by a nucleic acid sequence comprising two or more CAR sequences, joined by a sequence encoding a self-cleaving linker.

35. The chimeric antigen receptor of claim 34, wherein the self-cleaving linker has a sequence selected from the group consisting of: SEQ ID NO. 43; SEQ ID NO. 88 and SEQ ID NO. 89.

36. The chimeric antigen receptor of claim 34 or claim 35, wherein the CAR comprises: a CD8A extracellular signal peptide (SEQ ID NO: 39); a first antigen binding domain (SEQ ID NOS: 5, 6 or 7); a human CD8A hinge/transmembrane domain (SEQ ID NO: 34), a human CD137 co-stimulatory domain (SEQ ID NO: 33); and a human CD247 (CD3-zeta) intracellular signalling domain (SEQ ID NO: 32); a self-cleaving linker (SEQ ID NO: 43); a CD8A extracellular signal peptide (SEQ ID NO: 39); a second antigen binding domain of the antigen binding molecule of the first aspect (SEQ ID NOS: 17 or 18); a human TNFRSF4 hinge/transmembrane domain (SEQ ID NO: 35); a human CD137 co-stimulatory domain (SEQ ID NO: 33); and a human CD247 (CD3-zeta) intracellular signalling domain (SEQ ID NO: 32).

37. The chimeric antigen receptor of any one of claims 16 to 27 and 34 to 36, wherein the CAR has a sequence selected from the group consisting of: SEQ ID NOS: 44 to 46, 95, 96 and 102 to 106.

38. The chimeric antigen receptor of any one of claims 16 to 27 and 34 to 36, wherein the nucleic acid sequence is selected from the group consisting of: SEQ ID NOS: 83 to 85; 100 to 101 and 107 to 111

39. A recombinant T cell comprising the chimeric antigen receptor of any one of claims 16 to 38.

40. A nucleic acid having a sequence encoding the antigen binding molecule of any one of claims 1 to 14, or the chimeric antigen receptor any one of claims 16 to 39.

41. The nucleic acid of claim 40, wherein the sequence is selected from the group consisting of: SEQ ID NOs 73 to 85; 97 to 101 and 107 to 111.

42. The nucleic acid of claim 40 or claim 41, wherein the nucleic acid is a vector.

43. A vector comprising the nucleic acid of any one of claims 40 to 42.

44. A pharmaceutical composition comprising the chimeric antigen receptor of any one of claims 16 to 38, or the recombinant T cell of claim 39, or the nucleic acid of any one of claims 40 to 42, or the vector of claim 43, and a pharmaceutically acceptable carrier.

45. The antigen binding molecule of claims 1 to 14, or the chimeric antigen receptor of any one of claims 16 to 38, or the recombinant T cell of claim 39, or the nucleic acid of any one of claims 40 to 42, or the vector of claim 43; or the pharmaceutical composition of claim 44, for use as a medicament.

46. The antigen binding molecule of claims 1 to 14, or the chimeric antigen receptor of any one of claims 16 to 38, or the recombinant T cell of claim 39, or the nucleic acid of any one of claims 40 to 42, or the vector of claim 43; or the pharmaceutical composition of claim 44, for use in killing or inhibiting the growth of cells expressing CLL1 and/or CD33.

47. The antigen binding molecule of claims 1 to 14, or the chimeric antigen receptor of any one of claims 16 to 38, or the recombinant T cell of claim 39, or the nucleic acid of any one of claims 40 to 42, or the vector of claim 43; or the pharmaceutical composition of claim 44, for use in the treatment of a disease or disorder selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.

48. A method of treatment of a disorder or disease, wherein said method comprises administering to a patient in need thereof, antigen binding molecule of claims 1 to 14, or the chimeric antigen receptor of any one of claims 16 to 38, or the recombinant T cell of claim 39, or the nucleic acid of any one of claims 40 to 42, or the vector of claim 43; or the pharmaceutical composition of claim 44.

49. The method of treatment of claim 48, wherein the disorder or disease is selected from the group consisting of: cancers, such as AML, chronic myeloid (myelogenous) leukemia (CML), chronic myelomonocytic leukemia (CMML), juvenile myelomonocytic leukemia, atypical chronic myeloid leukemia, acute promyelocytic leukemia (APL), acute monocytic leukemia, acute monoblastic leukemia, acute erythroid leukemia, acute megakaryoblastic leukemia, myelodysplastic syndrome (MDS), myeloproliferative disorder, myeloid neoplasm, myeloid sarcoma), Blastic Plasmacytoid Dendritic Cell Neoplasm (BPDCN); autoimmune diseases such as rheumatoid arthritis, psoriasis, allergies, asthma, Crohn's disease, IBD, IBS, fibromyalga, mastocytosis, and Celiac disease, melanomas, and sarcomas.
